(19) United States
(12) Patent Application Publication
(10) Pub. No.: US 2023/0134301 A1
(43) Pub. Date: May 4, 2023

(54) ENGINEERED REGULATORY T CELL

(71) Applicant: KING'S COLLEGE LONDON, London (GB)

(72) Inventors: Marc MARTINEZ-LLORDELLA, London (GB); Alberto SANCHEZ-FUEYO, London (GB)

(21) Appl. No.: 17/639,056

(22) PCT Filed: Aug. 28, 2020
(86) PCT No.: PCT/GB2020/052076

§ 371 Date: Feb. 28, 2022

(30) Foreign Application Priority Data
Aug. 30, 2019 (GB) 1912515.2

(51) Int. Cl. A61K 35/17; C12N 5/0783; C07K 16/28; A61P 37/06; C07K 14/705; C07K 14/725; C07K 14/47

(52) U.S. Cl. CPC A61K 35/17; C12N 5/0637; C07K 16/2851; A61P 37/06; C07K 14/70521; C07K 14/7051; C07K 14/70517; C07K 14/4702; C12N 2502/30; C07K 2317/31; C07K 2317/569; C07K 2317/565; C07K 2317/53; C07K 2317/24; C07K 2319/03; C07K 2319/33

(57) ABSTRACT

The present invention provides an engineered regulatory T cell (Treg) comprising a chimeric antigen receptor (CAR), wherein the CAR comprises an antigen recognition domain which specifically binds to asialoglycoprotein receptor (ASGR). The present invention also provides a method of promoting liver tissue repair and/or regeneration in a subject which comprises the step of administering to the subject an engineered Treg comprising a CAR or a pharmaceutical composition comprising the engineered Treg, wherein the CAR comprises a liver-specific antigen recognition domain.

### FIELD OF THE INVENTION

The present invention relates to engineered regulatory T cells and therapeutic uses of such cells in the setting of immune-mediated liver damage. In particular, the invention relates to engineered regulatory T cells capable of recognizing liver cells, treating and/or preventing transplant rejection or immune-mediated damage, and promoting liver cell regeneration.

### BACKGROUND TO THE INVENTION

The liver is a vital organ that supports almost every other organ in the body. Due to its strategic location and important functions, the liver is prone to disease, particularly immune-mediated damage. Liver disease accounts for approximately 2 million deaths per year worldwide, 1 million due to complications of cirrhosis and 1 million due to viral hepatitis and hepatocellular carcinoma. Cirrhosis is currently the 11th most common cause of death globally. Cirrhosis is within the top 20 causes of disability-adjusted life years and years of life lost, accounting for 1.6% and 2.1% of the worldwide burden (Asrani, S. K., et al., 2018. Journal of hepatology, 70, pp. 151-171).

Common autoimmune liver diseases such as primary biliary cholangitis and primary sclerosing cholangitis may damage the liver and lead to cirrhosis. The burden of disease in primary biliary cholangitis is often manifested by poor quality of life, impaired health status and significant symptoms of fatigue, itching and depression. Primary sclerosing cholangitis is a risk factor for cholangiocarcinoma, gall bladder and colorectal cancer and may contribute to premature mortality. Compared to the general population, patients with primary sclerosing cholangitis have a 4-fold increased risk of mortality, but specifically have a 398-fold increased risk of developing cholangiocarcinoma (Asrani, S. K., et al., 2018. Journal of hepatology, 70, pp. 151-171).

Cirrhosis can also be caused by inflammatory liver disorders, such as viral hepatitis or steatohepatitis. In 2010, deaths from viral hepatitis accounted for 0.3 million deaths per year, an increase of 46% from 1990. Viral hepatitis increased from the 10th leading cause (1990) to the 7th leading cause of mortality in 2013. In 2015, viral hepatitis-related disease led to 1.34 million deaths, similar to the number caused by tuberculosis (1.37 million) and higher than the number caused by HIV (1.06 million deaths) or malaria (0.44 million deaths) (Asrani, S. K., et al., 2018. Journal of hepatology, 70, pp. 151-171).

In addition, graft tolerance and survival is an important issue during liver transplants. Acute cellular rejection occurs in 15-25% of liver transplant recipients on Tacrolimus based immunosuppression regimens and generally improves with steroids. While acute rejection usually responds well to treatment, chronic rejection represents a difficult situation and a significant proportion of patients do not respond to increased immunosuppression. Chronic rejection often leads to retransplantation or death (Choudhary, N. S., et al., 2017. Journal of clinical and experimental hepatology, 7(4), pp. 358-366). The five year mortality is lowest in patients given liver transplants for primary biliary cholangitis. Diseases that can recur include autoimmune hepatitis, primary biliary cirrhosis, and primary sclerosing cholangitis (Hirschfield, G. M., et al., 2009. BMJ, 338, p. b1670).

Liver transplants can also result in immune-mediated damage caused by graft-versus-host disease. The reported incidence of graft-versus-host disease varies from 0.1% to 2%, with a mortality rate of greater than 75% (Akbulut, S., et al., 2012. World journal of gastroenterology, 18(37), p. 5240).

The liver is endowed with unique regenerative properties, which can help ameliorate immune-mediated damage. However, in situations of chronic immune-mediated damage liver cells undergo senescence and their regenerative capacity is impaired, which contributes to the development of liver failure and causes significant patient morbidity and mortality (Aravinthan, A. D. and Alexander, G. J., 2016. Journal of hepatology, 65(4), pp. 825-834). Moreover, liver regeneration in response to immune-mediated damage can have adverse effects leading to cirrhosis and liver cancer (Michalopoulos, G. K., 2017. Hepatology, 65(4), pp. 1384-1392).

Consequently, a therapy capable of treating and/or preventing immune-mediated damage and promoting liver regeneration would have enormous therapeutic potential for the management of liver disease.

Accordingly, there is a need for therapies to treat and/or prevent immune-mediated damage and to promote liver regeneration.

### SUMMARY OF THE INVENTION

In a first aspect the present invention provides an engineered regulatory T cell (Treg) comprising a chimeric antigen receptor (CAR), wherein the CAR comprises an antigen recognition domain which specifically binds to asialoglycoprotein receptor (ASGR).

In some embodiments the CAR comprises an endodomain which comprises a STAT5 association motif and a JAK1- and/or a JAK2-binding motif. In some further embodiments the endodomain comprises a JAK3-binding motif and/or does not comprise a STAT3 association motif.

Suitably, the CAR endodomain does not comprise the amino acid sequence YXXQ (SEQ ID NO: 133). Suitably, the IL2Rβ portion of the CAR endodomain does not comprise the amino acid sequence YXXQ (SEQ ID NO: 133).

Engineered Tregs of the present invention with STAT5 signalling may be particularly effective in providing a survival advantage to the engineered CAR-Tregs after antigen recognition compared to the general T cell population of the subject. In particular, in the context of e.g. transplantation where the use of immunosuppressive drugs reduces the availability of IL-2, the STAT5 signalling of the CAR-Tregs provides additional survival and functional effects on the Tregs of the invention in an otherwise disadvantageous microenvironment.

In another aspect the present invention provides a pharmaceutical composition comprising an engineered Treg according to the present invention.

In another aspect the present invention provides an engineered Treg or pharmaceutical composition according to the present invention for use in induction of tolerance to a liver transplant in a subject, or for use in the treatment and/or prevention of liver transplant rejection, liver graft-versus-host disease (GvHD), an autoimmune liver disease, or an inflammatory liver disorder in a subject.

In another aspect the present invention provides a method of inducing tolerance to a liver transplant in a subject, or treating and/or preventing liver transplant rejection, liver graft-versus-host disease (GvHD), an autoimmune liver disease, or an inflammatory liver disorder in a subject, which comprises the step of administering to the subject an engineered Treg or a pharmaceutical composition according to the present invention.

In another aspect the present invention relates to use of an engineered Treg according to the present invention, for the manufacture of a medicament for inducing tolerance to a liver transplant in a subject, or for the manufacture of a medicament for treating and/or preventing liver transplant rejection, liver graft-versus-host disease (GvHD), an autoimmune liver disease, or an inflammatory liver disorder in a subject.

In another aspect the present invention provides an engineered regulatory T cell (Treg) comprising a chimeric antigen receptor (CAR) or a pharmaceutical composition comprising the engineered Treg, for use in repairing and/or regenerating liver tissue in a subject, wherein the CAR comprises a liver-specific antigen recognition domain.

In another aspect the present invention provides a method of promoting liver tissue repair and/or regeneration in a subject which comprises the step of administering to the subject an engineered Treg comprising a chimeric antigen receptor (CAR) or a pharmaceutical composition comprising the engineered Treg, wherein the CAR comprises a liver-specific antigen recognition domain.

In another aspect the present invention provides use of an engineered Treg comprising a chimeric antigen receptor (CAR) in the manufacture of a medicament for promoting liver tissue repair and/or regeneration in a subject, wherein the CAR comprises a liver-specific antigen recognition domain.

In another aspect the present invention provides an engineered regulatory T cell (Treg) comprising a chimeric antigen receptor (CAR) or a pharmaceutical composition comprising the engineered Treg, for use in treating and/or preventing liver cirrhosis, acute liver failure or acute-on-chronic liver failure in a subject, wherein the CAR comprises a liver-specific antigen recognition domain.

In another aspect the present invention provides a method of treating and/or preventing liver cirrhosis, acute liver failure or acute-on-chronic liver failure in a subject which comprises the step of administering to the subject an engineered Treg comprising a chimeric antigen receptor (CAR) or a pharmaceutical composition comprising the engineered Treg, wherein the CAR comprises a liver-specific antigen recognition domain.

In another aspect the present invention provides use of an engineered Treg comprising a chimeric antigen receptor (CAR) in the manufacture of a medicament for treating and/or preventing liver cirrhosis, acute liver failure or acute-on-chronic liver failure in a subject, wherein the CAR comprises a liver-specific antigen recognition domain.

The present invention further provides a chimeric antigen receptor (CAR) wherein the CAR comprises an antigen recognition domain which specifically binds to asialoglycoprotein receptor (ASGR) and which further comprises the CD3 zeta signalling domain.

In some embodiments the CAR comprises an endodomain which comprises a STAT5 association motif and a JAK1- and/or a JAK2-binding motif. In some further embodiments the endodomain comprises a JAK3-binding motif and/or does not comprise a STAT3 association motif. Thus, in one embodiment, the invention provides a CAR wherein the CAR comprises an antigen recognition domain which specifically binds to ASGR and which comprises a STAT5 association motif and a JAK1- and/or a JAK2-binding motif, and which preferably comprises a JAK3-binding motif and/or does not comprise a STAT3 association motif.

The present invention further provides a polynucleotide, a nucleic acid, or a vector encoding the CAR of the invention.

The present invention further provides a method of producing an engineered Treg according to the invention, comprising the following steps:
(i) isolation of a cell-containing sample from a subject or provision of a cell-containing sample; and
(ii) transduction or transfection of the cell-containing sample with a polynucleotide, a nucleic acid, or a vector encoding the CAR, to provide a population of engineered cells

wherein the cell-containing sample comprises Tregs and/or Tregs are enriched and/or generated from the cell-containing sample prior to or after step (ii).

### DESCRIPTION OF DRAWINGS

FIG. 1—Exemplary Designs of Anti-ASGR1 CAR Constructs

Schematics of exemplary anti-ASGR1 CAR constructs. (A) Anti-ASGR1 CAR construct comprising: ASGR1 VH antigen recognition domain; CD8α hinge domain; CD28 TM; CD28 signalling domain; CD3z signalling domain; P2A cleavage domain; and eGFP. (B) Anti-ASGR1 CAR construct comprising: ASGR1 VH antigen recognition domain; CD28 hinge domain; CD28 TM; CD28 signalling domain; CD3z signalling domain; P2A cleavage domain; and eGFP.

FIG. 2—Generation of Anti-ASGR1 CAR-Tregs

Histograms showing the generation of anti-ASGR1 CAR-Tregs as assessed by FACS. (A) Untransduced Tregs. (B) Transduced Tregs. Isolated CD4+CD25hiCD127− cells were isolated and activated with anti-CD3/CD28 beads. Two days after activation Tregs were transduced with lentivirus containing the ASGR1-CAR and the GFP reported gene. Transduced and untransduced Tregs were cultured during 10 days and GFP was measured to assess transduction efficacy.

FIG. 3—Validation of ASGR1 Expression on HepG2 Cell Line

Histograms showing the ASGR1 levels in K562 and HepG2 cells as assessed by FACS. (A) K562 cells. (B) HepG2 cells.

FIG. 4—Evaluation of the Antigen-Specificity of Anti-ASGR1 CAR-Tregs

Histograms showing the expression of CD69 in response to culture with (i) media alone; (ii) HepG2 cells; and (iii) anti-CD3/CD28 beads, as assessed by FACS. (A) Transduced Tregs (GFP+). (B) Transduced Tregs (GFP−). (C) Untransduced Tregs (GFP−). No upregulation of CD69 is observed in the presence of media alone. Following culture with HepG2 cells, only the GFP+ transduced Tregs (but not GPF-negative transduced cells or untransduced cells) upregulate CD69. In contrast, all cells upregulated CD69 when cultured with non-specific anti-CD3/CD28 bead stimulation.

FIG. 5—Evaluation of the Antigen-Specificity Suppressive Function of Anti-ASGR1 CAR-Tregs (1:1 Treg:Teff)

Histograms showing the proliferation of activated effector CD4+CD25− T cells as assessed by FACS. Suppression assay shows the impact of anti-ASGR1 CAR-Tregs on the proliferative capacity of effector T cells stained with Proliferation Dye and pre-activated with anti-CD3/CD28 beads. In response to HepG2 stimulation, anti-ASGR1 CAR Tregs were capable of effectively suppressing conventional T cell proliferation.

FIG. 6—Evaluation of the Antigen-Specificity Suppressive Function of Anti-ASGR1 CAR-Tregs (1:1, 1:2 and 1:5 Treg:Teff)

Suppression assay results for 1:1, 1:2 and 1:5 Treg:Teff. Suppression assay shows the impact of anti-ASGR1 CAR-Tregs on the proliferative capacity of effector T cells stained with Proliferation Dye and pre-activated with anti-CD3/CD28 beads. In response to HepG2 stimulation, anti-ASGR1 CAR Tregs were capable of effectively suppressing conventional T cell proliferation at a lower ratio of Treg:Teffector than untransduced Tregs.

FIG. 7—Exemplary Designs of Anti-HLA.A2 IL2R CAR Constructs

Schematics of exemplary anti-HLA.A2 CAR constructs including different combinations of IL2R endodomain. (A) dCAR construct: HLA.A2 scFv antigen recognition domain; CD28 hinge domain; CD28 TM and eGFP. (B) CD28z construct: HLA.A2 scFv antigen recognition domain; CD28 hinge domain; CD28 TM; CD28 signaling domain; CD3z signaling domain and eGFP. (C) IL2R Construct 1: HLA.A2 scFv antigen recognition domain; CD28 hinge domain; CD28 TM; CD28 signaling domain; truncated IL2RB endodomain; CD3z signaling domain and eGFP. (D) IL2R Construct 1: HLA.A2 scFv antigen recognition domain; CD28 hinge domain; CD28 TM; CD28 signaling domain; truncated IL2RG; truncated IL2RB endodomain; CD3z signaling domain and eGFP. (E) IL2R Construct 1: HLA.A2 scFv antigen recognition domain; CD28 hinge domain; CD28 TM; CD28 signaling domain; truncated IL2RB endodomain; CD3z signaling domain; FP2A cleavage domain and eGFP.

FIG. 8—Generation of Anti-HLA.A2 IL2R CAR-Tregs

Schematic illustration showing the generation and expansion of anti-HLA.A2 IL2R CAR-Tregs. (A) Isolated CD4+CD25hiCD127low cells were isolated and activated with anti-CD3/CD28 beads. Three days after activation Tregs were transduced with lentivirus containing the HLA.A2-CAR and the GFP reported gene. Fresh media and 1000 IU/ml IL-2 were added every 2 days. Transduced and untransduced Tregs were cultured during 10 days and GFP was measured to assess transduction efficacy. Tregs were further expanded with fresh anti-CD3/CD28 beads. (B) Fold change expansion of Tregs untransduced or transduced with different CAR constructs on day 10 after activation.

FIG. 9—Quantification of Transduction Efficacy of Anti-HLA.A2 IL2R Constructs Over Time

GFP expression was analysed on Tregs untransduced and transduced with CAR constructs at different time points after cell activation. (A) Representative contour plots of GFP expression from HLA-A2 IL2R CAR Tregs 7 days following transduction. (B) Quantification of GFP⁺ CAR Tregs among live CD4+ cells 7 days following transduction. (C) Quantification of GFP expression from HLA-A2 IL2R CAR Tregs over time.

FIG. 10—Quantification of Cell Surface Expression of Anti-HLA.A2 IL2R CAR Constructs on Transduced Tregs

Membrane expression of CAR construct on untransduced and transduced Tregs was analysed by PE-conjugated HLA-A*0201/CINGVCWTV dextramers (Immudex, Copenhagen, Denmark). (A) Representative contour plots of GFP+Dextramer+ CAR Tregs 7 days following transduction. (B) Quantification of Dextramer+ cells among the GFP+ Tregs on day 7 after transduction.

FIG. 11—Phenotypic Characterization of CAR Tregs After Polyclonal Cell Expansion

Tregs were cultured and expanded for 15 days in the presence of anti-CD3/CD28 activation beads and IL-2. Treg related markers FOXP3, HELIOS, CTLA4 and TIGIT were analysed by FACS on untransduced and transduced Tregs to assess phenotypic lineage stability on day 15 of culture.

FIG. 12—Evaluation of the Antigen-Specificity of Anti-HLA.A2 IL2R CAR Tregs

Untransduced and transduced Tregs were cultured for 18 hours in the presence of different stimulus. CD69 and CD137 activation markers were analysed to assess specific and unspecific cell activation. (A) Representative contour plots showing the expression CD69 in response to culture with K562 cells transduced with HLA.A1 or HLA.A2 molecules. GFP signal was used to select the transduced Tregs. (B) Quantification of CD69 and CD137 expression on Tregs 18 hours after culture with media alone (unstimulated), anti-CD3/CD28 beads (unspecific stimulation), K562-HLA.A1 and K562-HLA.A2 cells. (C) Representative histograms showing CD69 expression on Tregs after 18 hours culture with HLA.A1 and HLA.A2 B cell lines. Different cell to cell ratios were used.

FIG. 13—STAT5 Phosphorylation Analysis as an Indicator of IL2R CAR Signaling

Transduced CAR Tregs were rested overnight in culture media without IL2. STAT5 phosphorylation of Tregs was assessed by FACS analysis 10 and 120 minutes after culture with media alone, 1000 IU/ml IL-2 or in the presence of HLA.A2-Ig based artificial APCs (produced following the protocol described at DOI: 10.3791/2801). (A) Contour plots showing the expression of GFP and phosphoSTAT5 on transduced CAR-Tregs after 10 minutes culture with media alone, HLA.A2 beads at 1:1 ratio and 1000 IU/ml IL-2. (B) Histograms showing the phosphorylation of STAT5 of Tregs cultured for 120 minutes with HLA.A2 beads 1:1 ratio or media alone (unstim).

FIG. 14—Evaluation of Treg Survival After Unspecific and HLA.A2 Specific Activation in the Absence of IL-2

CAR transduced Tregs with different constructs were cultured with anti-CD3/28 activation beads and K562.A2 expression cells without the presence of IL-2. Cell survival was assessed 7 days after activation by FACS analysis. (A) Representatives histograms of CAR-Tregs showing cell survival of GFP+ cells based on Viability dye statining on day 7 after activation without IL-2. (B) Percentage of viable cells on GFP+ Tregs after 7 days of culture whit anti-CD3/28 beads and K562-HLA.A2 cells in absence of IL-2 (* p<0.05, ANOVA analysis with Tukey's post hoc correction).

FIG. 15—Treg Suppression Potency Test: Evaluate the Immunoregulatory Function of Tregs by Analysing the Modulation of Co-Stimulatory Molecules on B Cells

B cell expression of CD80 and CD86 after co-culture with Tregs was analysed to evaluate the capacity of Tregs to reduce the expression of co-stimulatory molecules on antigen presenting cells. Fixed number of alive A2-expressing B cells (20 K/well) were co-cultured with titrated numbers of Treg products (A2-negative donors) (200, 100, 50, 25, 12.5K) overnight. FACS analysis of CD86 and CD80 co-stimulatory markers on B cells.

FIG. 16—Evaluation of the Effect of Pre-Activated Non-Transduced Tregs on Albumin Production by Primary Hepatocytes In Vitro

Primary hepatocytes were cultured alone or in the presence of previously activated regulatory T cells for 7 days. The addition of regulatory T cells resulted in improved albumin secretion detectable in the supernatant. Similar effects were noted when hepatocytes were cultured without regulatory T cells but in the presence of conditioned media obtained from cultures containing activated regulatory T cells. This Figure shows that the addition of pre-activated non-transduced Tregs increases the production of albumin by primary hepatocytes in vitro.

FIG. 17—Evaluation of the Effect of Pre-Activated Non-Transduced Tregs and Effector T Cells on Albumin Production by Primary Hepatocytes In Vitro

Primary hepatocytes were cultured alone or in the presence of previously activated regulatory T, pre-activated effector T cells or both, for 3 days. The addition of regulatory T cells resulted in improved albumin secretion detectable in the supernatant. This was not observed when hepatocytes were cultured with effector T cells, which resulted in trend towards reduced albumin levels. The favorable effect of regulatory T cells on albumin levels were observed even when regulatory T cells were combined with pre-activated effector T cells. This Figure shows that the addition of pre-activated non-transduced Tregs, but not of effector T cells, increases the production of albumin by primary hepatocytes in vitro.

FIG. 18—Evaluation of the Effect of Anti-ASGPR CAR Tregs on Albumin Production by Primary Hepatocytes In Vitro

Primary hepatocytes were cultured alone or in the presence of resting un-manipulated regulatory T cells or resting regulatory T cells lentivirally transduced to express an anti-ASGPR CAR. Only Tregs bearing the anti-ASGPR and therefore capable of undergoing antigen-specific activation in response to ASGPR expressed by hepatocytes exerted a significant effect on albumin levels in the supernatant. This Figure shows that the addition of non pre-activated Tregs expressing an anti-ASGPR CAR increases the production of albumin by primary hepatocytes in vitro, beyond what is achieved when non-transduced Tregs are employed.

### DETAILED DESCRIPTION

The present invention provides an engineered Treg comprising a CAR, which CAR provides an activatory signal to the Treg exclusively upon CAR binding to a liver specific antigen (e.g. ASGR); and thus enhances the retention, function and the survival of the engineered Treg specifically within the liver microenvironment.

The engineered Treg of the invention may, upon binding to a liver-specific antigen (e.g. ASGR), increase or improve liver regeneration and tissue repair.

Various preferred features and embodiments of the present invention will now be described by way of non-limiting examples.

It must be noted that as used herein and in the appended claims, the singular forms “a”, “an”, and “the” include plural referents unless the context clearly dictates otherwise.

The terms “comprising”, “comprises” and “comprised of” as used herein are synonymous with “including”, “includes” or “containing”, “contains”, and are inclusive or open-ended and do not exclude additional, non-recited members, elements or method steps. The terms “comprising”, “comprises” and “comprised of” also include the term “consisting of”.

The publications discussed herein are provided solely for their disclosure prior to the filing date of the present application. Nothing herein is to be construed as an admission that such publications constitute prior art to the claims appended hereto.

This disclosure is not limited by the exemplary methods and materials disclosed herein, and any methods and materials similar or equivalent to those described herein can be used in the practice or testing of embodiments of this disclosure. Numeric ranges are inclusive of the numbers defining the range. Unless otherwise indicated, any nucleic acid sequences are written left to right in 5′ to 3′ orientation; amino acid sequences are written left to right in amino to carboxy orientation, respectively.

Engineered Regulatory T Cell (Treg)

Regulatory T cells (Treg) are immune cells with suppressive function that control cytopathic immune responses and are essential for the maintenance of immunological tolerance.

As used herein, the term Treg refers to a T cell with immunosuppressive function.

Suitably, “immunosuppressive function” may refer to the ability of the Treg to reduce or inhibit one or more of a number of physiological and cellular effects facilitated by the immune system in response to a stimulus such as a pathogen, antigen, e.g. an alloantigen, or an autoantigen. Examples of such effects include increased proliferation of conventional T cells (Tconv) and secretion of pro-inflammatory cytokines. Any such effects may be used as indicators of the strength of an immune response. A relatively weaker immune response by Tconv in the presence of Tregs would indicate an ability of the Treg to suppress immune responses. For example, a relative decrease in cytokine secretion would be indicative of a weaker immune response, and thus indicative of the ability of Tregs to suppress immune responses. Tregs can also suppress immune responses by modulating the expression of co-stimulatory molecules on antigen presenting cells (APCs), such as B cells, dendritic cells and macrophages. Expression levels of CD80 and CD86 can be used to assess suppression potency of activated Tregs in vitro after co-culture.

Assays are known in the art for measuring indicators of immune response strength, and thereby the suppressive ability of Tregs. In particular, antigen-specific Tconv cells may be co-cultured with Tregs, and a peptide of the corresponding antigen added to the co-culture to stimulate a response from the Tconv cells. The degree of proliferation of the Tconv cells and/or the quantity of the cytokine IL-2 they secrete in response to addition of the peptide may be used as indicators of the suppressive abilities of the co-cultured Tregs.

Antigen-specific Tconv cells co-cultured with Tregs of the present invention may proliferate 5%, 10%, 20%, 30%, 40%, 50%, 60%, 70%, 90%, 95% or 99% less than the same Tconv cells cultured in the absence of Tregs of the invention.

Antigen-specific Tconv cells co-cultured with Tregs of the invention may express at least 10%, at least 20%, at least 30%, at least 40%, at least 50%, or at least 60% less effector cytokine than corresponding Tconv cells cultured in the absence of Tregs of the invention.

The effector cytokine may be selected from IL-2, IL-17, TNFα, GM-CSF, IFN-γ, IL-4, IL-5, IL-9, IL-10 and IL-13.

Suitably the effector cytokine may be selected from IL-2, IL-17, TNFα, GM-CSF and IFN-γ.

Suitably, the Treg is a T cell which expresses the markers CD4, CD25 and FOXP3 (CD4⁺CD25⁺FOXP3⁺). “FOXP3” is the abbreviated name of the forkhead box P3 protein. FOXP3 is a member of the FOX protein family of transcription factors and functions as a master regulator of the regulatory pathway in the development and function of regulatory T cells.

Marker levels can be determined by any method known to those of skill in the art, for example flow cytometry.

Tregs may also express CTLA-4 (cytotoxic T-lymphocyte associated molecule-4) and/or GITR (glucocorticoid-induced TNF receptor). Treg cells are present in the peripheral blood, lymph nodes, and tissues.

Suitably, the Treg may be identified using the cell surface markers CD4 and CD25 in the absence of or in combination with low-level expression of the surface protein CD127 (CD4⁺CD25⁺CD127⁻ or CD4⁺CD25⁺CD127^{low} or CD4⁺CD25^{hi}CD127⁻ or CD4⁺CD25^{hi}CD127^{low}). The use of such markers to identify Tregs is known in the art and described in Liu et al. (JEM; 2006; 203; 7(10); 1701-1711), for example.

The Treg may be a CD4⁺CD25⁺FOXP3⁺ T cell or a CD4⁺CD25^{hi}FOXP3⁺ T cell.

The Treg may be a CD4⁺CD25⁺CD127⁻ T cell or a CD4⁺CD25^{hi}CD127⁻ T cell.

The Treg may be a CD4⁺CD25⁺FOXP3⁺CD127⁻ T cell or a CD4⁺CD25^{hi}FOXP3⁺CD127⁻ T cell.

The Treg may have a demethylated Treg-specific demethylated region (TSDR). The TSDR is an important methylation-sensitive element regulating FOXP3 expression (Polansky, J. K., et al., 2008. European journal of immunology, 38(6), pp. 1654-1663).

The Treg may be natural or thymus-derived, adaptive or peripherally-derived, or in vitro-induced (Abbas, A. K., et al., 2013. Nature immunology, 14(4), p. 307-308). Suitably, the Treg may be CD4⁺CD25⁺FOXP3⁺Helios⁺Neuropilin 1⁺.

Further suitable Tregs include, but are not limited to, Tr1 cells (which do not express Foxp3, and have high IL-10 production); CD8⁺FOXP3⁺ T cells; and γδ FOXP3⁺ T cells.

Suitably, the Treg is isolated from peripheral blood mononuclear cells (PBMCs) obtained from a subject. Suitably the subject is a mammal, preferably a human.

Suitably, the Treg is matched (e.g. HLA-matched) or is autologous to a subject to whom the engineered Treg is to be administered. Suitably, the subject to whom the engineered Treg is to be administered is a mammal, preferably a human. The Treg may be generated ex vivo either from a patient's own peripheral blood (1st party), or in the setting of a haematopoietic stem cell transplant from donor peripheral blood (2nd party), or peripheral blood from an unconnected donor (3rd party). Suitably the Treg is autologous to the subject to whom the engineered Treg is to be administered.

In a preferred embodiment, the Treg is isolated from peripheral blood mononuclear cells (PBMCs) obtained from a subject and is matched (e.g. HLA-matched) or is autologous to the subject to whom the engineered Treg is to be administered.

Suitably, the Treg is part of a population of Tregs. Suitably, the population of Tregs comprises at least 70% Tregs, such as at least 75, 85, 90, 95, 97, 98 or 99% Tregs. Such a population may be referred to as an “enriched Treg population” or an “enriched Treg sample”.

In some aspects, the Treg may be derived from ex-vivo differentiation of inducible progenitor cells or embryonic progenitor cells to the Treg.

As used herein, the term “conventional T cell” or Tcon means a T lymphocyte cell which expresses an αβ T cell receptor (TCR) as well as a co-receptor which may be cluster of differentiation 4 (CD4) or cluster of differentiation 8 (CD8) and which does not have an immunosuppressive function. Conventional T cells are present in the peripheral blood, lymph nodes, and tissues. Suitably, the engineered Treg may generated from a Tcon by introducing DNA or RNA coding for FOXP3 in addition to the DNA or RNA coding for the CAR as described herein, by one of many means including transduction with a viral vector, or transfection with DNA or RNA, on the same or different vectors. Alternatively, the engineered Treg may be generated from a Tcon by in vitro culture of CD4⁺CD25⁻FOXP3⁻ cells in the presence of IL-2 and TGF-β.

The Treg may be derived from ex-vivo differentiation of inducible progenitor cells or embryonic progenitor cells to the Treg. A polynucleotide or vector of the invention may introduced into the inducible progenitor cells or embryonic progenitor cells prior to, or after, differentiation to a Treg.

An “engineered Treg” as used herein means a Treg which has been modified to comprise or express a polynucleotide which is not naturally encoded by the cell, in particular a CAR as described herein. Methods for engineering Tregs are known in the art and include, but are not limited to, genetic modification of Tregs e.g. by transduction such as retroviral or lentiviral transduction, transfection (such as transient transfection, DNA or RNA based) including lipofection, polyethylene glycol, calcium phosphate and electroporation. Any suitable method may be used to introduce a nucleic acid sequence into a Treg.

Chimeric Antigen Receptor

“Chimeric antigen receptor” or “CAR” or “CARs” as used herein refers to engineered receptors which confer an antigen specificity onto cells, in this case Tregs. CARs are also known as artificial T-cell receptors, chimeric T-cell receptors or chimeric immunoreceptors. The CARs of the invention comprise a binding domain specific for a liver antigen (e.g. ASGR), optionally a hinge domain, a transmembrane domain, and an endodomain (comprising an intracellular signalling domain and optionally one or more co-stimulatory domains). Typically, CARs of the invention may be expressed or present as a single polypeptide chain, e.g. a single polypeptide chain comprising a binding domain specific for a liver antigen (e.g. ASGR), optionally a hinge domain, a transmembrane domain and an endodomain (comprising an intracellular signalling domain and optionally one or more co-stimulatory domains). Particularly, typically, the intracellular signalling domain and optionally one or more co-stimulatory domains may be present with a single polypeptide chain. Thus, although, it is possible for CARs of the invention to comprise more than one polypeptide chain, e.g. to comprise a dual polypeptide or CAR system, in a particular embodiment, the CAR of the invention is not a dual CAR.

CAR-encoding polynucleotides may be transferred to the Treg using, for example, retroviral vectors. In this way, a large number of antigen-specific T cells can be generated for adoptive cell transfer. When the CAR binds the target-antigen, this results in the transmission of an activating signal to the Treg it is expressed on. Thus, the CAR directs the engineered Treg towards cells expressing the targeted antigen.

A Treg of the invention may comprise one or more different CARs of the invention, and may additionally comprise exogenous polynucleotides encoding other polypeptides.

Antigen Recognition Domain

The CAR of the invention comprises an antigen recognition domain.

The “antigen recognition domain” as used herein refers to the extracellular part of the CAR that defines the antigen-binding capability of the CAR. In certain aspects of the invention, the antigen recognition domain provides the CAR with the ability to bind to a liver-specific antigen, preferably asialoglycoprotein receptor (ASGR). Thus, the antigen recognition domain targets a liver-specific antigen, preferably ASGR.

A liver-specific antigen is one which is preferentially expressed in liver tissue e.g. in hepatocytes, parenchymal cells, kupffer cells, stellate cells, and/or liver sinusoidal endothelial cells. Suitably, a liver-specific antigen is an antigen which has higher expression levels in liver tissue (e.g. in at least one of hepatocytes, parenchymal cells, kupffer cells, stellate cells, and liver sinusoidal endothelial cells) as compared to other tissues. For example, a liver-specific antigen may be an antigen which has expression levels at least 10% higher, at least 20% higher, at least 30% higher, at least 40% higher, at least 50% higher, at least 100% higher, at least 200% higher, at least 300% higher, at least 400% higher, at least 500% higher, or at least 1000% higher compared to expression levels in other tissues. Preferably, a liver specific-antigen is one which is only expressed in liver tissue, i.e. is not detectably expressed in other tissues.

The liver-specific antigen will be accessible to Treg cells (although it will be appreciated that the liver-specific antigen will not need to be accessible upon every liver cell type expressing the antigen), for instance the liver-specific antigen may be expressed on the cell surface.

Examples of suitable liver-specific antigens include asialoglycoprotein receptor (ASGR), sodium/taurocholate cotransporting polypeptide (NTCP), mannose-6-phosphate receptor, type VI collagen receptor, PDGF receptor, scavenger receptor class A, scavenger receptor class B type I, heparan sulfate, glycyrrhizin receptors, HDL-receptor, LDL-receptor, transferrin receptor, insulin receptor, alpha-2-macroglobulin receptor, ferritin receptor, uroplasminogen receptor, and thrombin receptor.

Preferably, the antigen recognition domain specifically binds to asialoglycoprotein receptor (ASGR). Most preferably, the antigen recognition domain binds to human ASGR. In some embodiments, the antigen recognition domain binds to both human ASGR and ASGR from other animals, e.g. mouse ASGR. Suitably, the antigen recognition domain may bind specifically to human ASGR.

Asialoglycoprotein receptor (ASGR or ASGPR) is a C-type lectin, primary expressed on the sinusoidal surface of the hepatocyte. ASGR is formed by a major 48 kDa subunit (ASGR1) and a minor 40 kDa subunit (ASGR2). The major role of ASGR is the binding, internalization, and subsequent clearance from the circulation of glycoproteins that contain terminal galactose or N-acetylgalactosamine residues (asialoglycoproteins). (Roggenbuck, D., et al., 2012. Autoimmunity Highlights, 3(3), p. 119). In normal hepatocytes, ASGR is expressed in a polar manner on the sinusoidal and basolateral surface of the plasma hepatocyte membrane. However, during liver inflammation, ASGR's expression shifts towards the canalicular membrane. In end-stage liver disease (cirrhosis), ASGR is over-expressed and serum levels of asialoglycoproteins are increased. (Roggenbuck, D., et al., 2012. Autoimmunity Highlights, 3(3), p. 119).

The antigen recognition domain may bind to ASGR1 and/or ASGR2, preferably ASGR1. The antigen recognition domain may specifically bind to ASGR1 and/or ASGR2, preferably ASGR1.

Human ASGR1 (UniProt entry P07306) is encoded by the ASGR1 gene. Spliced transcript variants encoding multiple isoforms have been observed for this gene (Harris, R. L., et al., 2012. Molecular biology international, 2012, Article ID 283974, 10 pages). The longer transcript contains all 8 exons, is by far the more abundant, and encodes full-length ASGR1 (isoform a, 291 amino acids). The shorter transcript has an in-frame deletion of exon 3 resulting in the loss of 39 residues (isoform b, 252 amino acids). Isoform b lacks the transmembrane domain and is secreted as a soluble protein. Illustrative sequences of human ASGR1 isoforms a and b are shown below (SEQ ID NOs: 1 and 2 respectively).

SEQ ID NO: 1
Human ASGR1 isoform a (NP_001662.1)
MTKEYQDLQHLDNEESDHHQLRKGPPPPQPLLQRLCSGPRLLLLSLGLSL
LLLVVVCVIGSQNSQLQEELRGLRETFSNFTASTEAQVKGLSTQGGNVGR
KMKSLESQLEKQQKDLSEDHSSLLLHVKQFVSDLRSLSCQMAALQGNGSE
RTCCPVNWEHERSCYWFSRSGKAWADADNYCRLEDAHLVVVTSWEEQKFV
QHHIGPVNTWMGLHDQNGPWKWWDGTDYETGFKNWRPEQPDDWYGHGLGG
GEDCAHFTDDGRWNDDVCQRPYRWCETELDKASQEPPLL
SEQ ID NO: 2
Human ASGR1 isoform b (NP_001184145.1)
MTKEYQDLQHLDNEESDHHQLRKDSQLQEELRGLRETFSNFTASTEAQVK
GLSTQGGNVGRKMKSLESQLEKQQKDLSEDHSSLLLHVKQFVSDLRSLSC
QMAALQGNGSERTCCPVNWVEHERSCYWFSRSGKAWADADNYCRLEDAHL
VVVTSWEEQKFVQHHIGPVNTWMGLHDQNGPWKWVDGTDYETGFKNWRPE
QPDDWYGHGLGGGEDCAHFTDDGRWNDDVCQRPYRWVCETELDKASQEPP
LL

The antigen recognition domain may bind, suitably specifically bind, to human ASGR1 isoform a and/or human ASGR1 isoform b, preferably human ASGR1 isoform a. The antigen recognition domain may bind, suitably specifically bind, to one or more polypeptides with at least 80%, 85%, 90%, 95%, 98%, 99%, or 100% identity to SEQ ID NO: 1 and/or SEQ ID NO: 2. Preferably the antigen recognition domain binds, suitably specifically binds, to a polypeptide with at least 80%, 85%, 90%, 95%, 98%, 99%, or 100% identity to SEQ ID NO: 1.

Human ASGR2 (UniProt entry P07307) is encoded by the ASGR2 gene. Alternatively spliced transcript variants encoding multiple isoforms have been observed for this gene (Harris, R. L., et al., 2012. Molecular biology international, 2012, Article ID 283974, 10 pages). ASGR2 gives rise to five transcripts (TH2′, T1, T2, T3, and T4) encoding four isoforms (a to d) that contain different in-frame deletions arising from alternative exon splicing events. Isoforms a and c contain 5 amino acids that serve as a proteolysis cleavage signal near the junction between the transmembrane domain and the CRD. Isoforms b and d lack this signal therefore they are not proteolytically cleaved but rather remain membrane bound where they may oligomerize with ASGR1 isoform a, to form native ASGR at the cell surface. Illustrative sequences of ASGR2 isoforms a-d are shown below (SEQ ID NOs: 3-6).

SEQ ID NO: 3
Human ASGR2 isoform a (NP_001172.1 and NP_550434.
1)
MAKDFQDIQQLSSEENDHPFHQGEGPGTRRLNPRRGNPFLKGPPPAQPLA
QRLCSMVCFSLLALSFNILLLVVICVTGSQSEGHRGAQLQAELRSLKEAF
SNFSSSTLTEVQAISTHGGSVGDKITSLGAKLEKQQQDLKADHDALLFHL
KHFPVDLRFVACQMELLHSNGSQRTCCPVNWVEHQGSCYWFSHSGKAWAE
AEKYCQLENAHLVVINSWEEQKFIVQHTNPFNTWIGLTDSDGSWKWVDGT
DYRHNYKNWAVTQPDNWHGHELGGSEDCVEVQPDGRWNDDFCLQVYRWVC
EKRRNATGEVA
SEQ ID NO: 4
Human ASGR2 isoform b (NP_550435.1)
MAKDFQDIQQLSSEENDHPFHQGPPPAQPLAQRLCSMVCFSLLALSFNIL
LLVVICVTGSQSAQLQAELRSLKEAFSNFSSSTLTEVQAISTHGGSVGDK
ITSLGAKLEKQQQDLKADHDALLFHLKHFPVDLRFVACQMELLHSNGSQR
TCCPVNWVEHQGSCYWFSHSGKAWAEAEKYCQLENAHLVVINSWEEQKFI
VQHTNPFNTWIGLTDSDGSWKWDGTDYRHNYKNWAVTQPDNWHGHELGGS
EDCVEVQPDGRWNDDFCLQVYRWVCEKRRNATGEVA
SEQ ID NO: 5
Human ASGR2 isoform c (NP_550436.1)
MAKDFQDIQQLSSEENDHPFHQGPPPAQPLAQRLCSMVCFSLLALSFNIL
LLVVICVTGSQSEGHRGAQLQAELRSLKEAFSNFSSSTLTEVQAISTHGG
SVGDKITSLGAKLEKQQQDLKADHDALLFHLKHFPVDLRFVACQMELLHS
NGSQRTCCPVNWVEHQGSCYWFSHSGKAWAEAEKYCQLENAHLVVINSWE
EQKFIVQHTNPFNTWIGLTDSDGSWKWVDGTDYRHNYKNWAVTQPDNWHG
HELGGSEDCVEVQPDGRWNDDFCLQVYRWVCEKRRNATGEVA
SEQ ID NO: 6
Human ASGR2 isoform d (NP_001188281.1)
MAKDFQDIQQLSSEENDHPFHQGEGPGTRRLNPRRGNPFLKGPPPAQPLA
QRLCSMVCFSLLALSFNILLLVVICVTGSQSAQLQAELRSLKEAFSNFSS
STLTEVQAISTHGGSVGDKITSLGAKLEKQQQDLKADHDALLFHLKHFPV
DLRFVACQMELLHSNGSQRTCCPVNWVEHQGSCYWFSHSGKAWAEAEKYC
QLENAHLVVINSWEEQKFIVQHTNPFNTWIGLTDSDGSWKWVDGTDYRHN
YKNWAVTQPDNWHGHELGGSEDCVEVQPDGRWNDDFCLQVYRWVCEKRRN
ATGEVA

The antigen recognition domain may bind, suitably specifically bind, to human ASGR2 isoform a, b, c and/or d, preferably human ASGR2 isoforms b and/or d. The antigen recognition domain may bind, suitably specifically bind, to one or more polypeptides with at least 80%, 85%, 90%, 95%, 98%, 99%, or 100% identity to one or more of SEQ ID NOs: 3-6. Preferably the antigen recognition domain binds, suitably specifically binds, to a polypeptide with at least 80%, 85%, 90%, 95%, 98%, 99%, or 100% identity to SEQ ID NO: 4 or with at least 80%, 85%, 90%, 95%, 98%, 99%, or 100% identity to SEQ ID NO: 6.

The antigen recognition domain may bind, suitably specifically bind, to: (a) human ASGR comprising human ASGR1 isoform a and human ASGR2 isoform b; and/or (b) human ASGR comprising human ASGR1 isoform a and human ASGR2 isoform d. The antigen recognition domain may bind, suitably specifically bind, to: (a) one or more polypeptide oligomers comprising: (i) one or more polypeptides with at least 80%, 85%, 90%, 95%, 98%, 99%, or 100% identity to SEQ ID NO: 1; and (ii) one or more polypeptides with at least 80%, 85%, 90%, 95%, 98%, 99%, or 100% identity to SEQ ID NO: 4; and/or (b) one or more polypeptide oligomers comprising: (i) one or more polypeptides with at least 80%, 85%, 90%, 95%, 98%, 99%, or 100% identity to SEQ ID NO: 1; and (ii) one or more polypeptides with at least 80%, 85%, 90%, 95%, 98%, 99%, or 100% identity to SEQ ID NO: 6.

In some embodiments, the antigen recognition domain binds to both human ASGR1 and/or ASGR2 and ASGR1 and/or ASGR2 from other animals, for example mouse ASGR1 and/or mouse ASGR2. Thus, in some embodiments the antigen recognition domain shows cross reactivity between human ASGR and mouse ASGR. For instance, if the antigen recognition domain binds to human ASGR1 isoform a, in some embodiments it also binds to mouse ASGR1 isoform a. Illustrative mouse ASGR1 isoforms a and b, and mouse ASGR2 isoforms a and b are shown below (SEQ ID NOs: 7-10).

SEQ ID NO: 7
Mouse ASGR1 isoform a (NP_033844.1 and NP_
001278060.1)
MTKDYQDFQHLDNDNDHHQLRRGPPPTPRLLQRLCSGSRLLLLSSSLSIL
LLVVVCVITSQNSQLREDLLALRQNFSNLTVSTEDQVKALSTQGSSVGRK
MKLVESKLEKQQKDLTEDHSSLLLHVKQLVSDVRSLSCQMAAFRGNGSER
TCCPINWVEYEGSCYWFSSSVRPWTEADKYCQLENAHLVVVTSRDEQNFL
QRHMGPLNTWIGLTDQNGPWKWVDGTDYETGFQNWRPEQPDNWYGHGLGG
GEDCAHFTTDGRWNDDVCRRPYRWVCETKLDKAN
SEQ ID NO: 8
Mouse ASGR1 isoform b (NP_001278061.1)
MTKDYQDFQHLDNDNDHHQLRRGPPPTPRLLQRLCSGSRLLLLSSSLSIL
LLVVVCVITSQNSQLREDLLALRQNFSNLTVSTEDQVKALSTQGSSVGRK
MKLVESKLEKQQKDLTEGSERTCCPINWVEYEGSCYWFSSSVRPWTEADK
YCQLENAHLVVVTSRDEQNFLQRHMGPLNTWIGLTDQNGPWKWVDGTDYE
TGFQNWRPEQPDNWYGHGLGGGEDCAHFTTDGRWNDDVCRRPYRWVCETK
LDKAN
SEQ ID NO: 9
Mouse ASGR2 isoform a (NP_031519.1, NP_001300854.
1, and NP_001300855.1)
MEKDCQDIQQLDSEENDHQLSGDDEHGSHVQDPRIENPHWKGQPLSRPFP
QRLCSTFRLSLLALAFNILLLVVICVVSSQSIQLQEEFRTLKETFSNFSS
STLMEFGALDTLGGSTNAILTSWLAQLEEKQQQLKADHSTLLFHLKHFPM
DLRTLTCQLAYFQSNGTECCPVNWVEFGGSCYWFSRDGLTWAEADQYCQL
ENAHLLVINSREEQDFVVKHRSQFHIWIGLTDRDGSWKWVDGTDYRSNYR
NWAFTQPDNWQGHEQGGGEDCAEILSDGHWNDNFCQQVNRWVCEKRRNIT
H
SEQ ID NO: 10
Mouse ASGR2 isoform b (NP_001300856.1)
MEFGALDTLGGSTNAILTSWLAQLEEKQQQLKADHSTLLFHLKHFPMDLR
TLTCQLAYFQSNGTECCPVNWVEFGGSCYWFSRDGLTWAEADQYCQLENA
HLLVINSREEQDFVVKHRSQFHIWIGLTDRDGSWKWVDGTDYRSNYRNWA
FTQPDNWQGHEQGGGEDCAEILSDGHWNDNFCQQVNRVWVCEKRRNITH

The antigen recognition domain may bind, suitably specifically bind, to mouse ASGR1 isoform a, mouse ASGR1 isoform b, mouse ASGR2 isoform a, and/or mouse ASGR2 isoform b, preferably mouse ASGR1 isoform a. The antigen recognition domain may bind, suitably specifically bind, to the equivalent human protein. The antigen recognition domain may bind, suitably specifically bind, to one or more polypeptides with at least 80%, 85%, 90%, 95%, 98%, 99%, or 100% identity to one or more of SEQ ID NOs: 7-10. Preferably the antigen recognition domain binds, suitably specifically binds, to a polypeptide with at least 80%, 85%, 90%, 95%, 98%, 99%, or 100% identity to SEQ ID NO: 1 and with at least 80%, 85%, 90%, 95%, 98%, 99%, or 100% identity to SEQ ID NO: 7.

The antigen recognition domain may bind, suitably specifically bind, one or more region or epitope within the liver-specific antigen, e.g. ASGR. An epitope, also known as antigenic determinant, is the part of an antigen that is recognised by an antigen recognition domain (e.g. an antibody). In other words, the epitope is the specific piece of the antigen to which an antigen recognition domain binds. Suitably, the antigen recognition domain binds, suitably specifically binds, to one region or epitope within the liver-specific antigen, e.g. ASGR.

The antigen recognition domain used in the present invention may selectively or specifically bind to a liver-specific antigen, preferably ASGR, and thus may have a greater binding affinity for a liver-specific antigen, preferably ASGR, as compared to its binding affinity for other proteins/molecules. Suitably, “specifically binds” as used herein means that the antigen recognition domain does not bind to other proteins or binds with a greatly reduced affinity compared to the binding to the antigen to which it specifically binds (e.g. with an affinity of at least 10, 50, 100, 500, 1000 or 10000 times less than its affinity for the antigen to which it specifically binds). Thus, the antigen recognition domain as referred to herein may bind to its antigen, e.g. ASGR with at least 10, 50, 100, 500, 1000 or 10000 times the affinity of its binding to other proteins. The binding affinity of the antigen recognition domain can be determined using methods well known in the art such as with the Biacore system.

The antigen recognition domain may have a high binding affinity for the liver-specific antigen, e.g. ASGR i.e. may have a Kd in the range of 10⁻⁶M or less, 10⁻⁷M or less, 10⁻⁸M or less, 10⁻⁹M or less, 10⁻¹⁰M or less, 10⁻¹¹M or less, or 10⁻¹²M or less. The antigen recognition domain may have a binding affinity for the liver-specific antigen, e.g. ASGR that corresponds to a Kd of less than 100 nM, 50 nM, 20 nM or 10 nM, more preferably of less than 10, 9.5, 9, 8.5, 8, 7.5, 7, 6.5, 6, 5.5, 5, 4.5, 4, 3.5, 3, 2.5, 2, 1.5 or 1 nM, most preferably less than 0.9, 0.8, 0.7, 0.6, 0.5, 0.4, 0.3, 0.2 or 0.1 nM. The antigen recognition domain may have a binding affinity for the liver-specific antigen, e.g. ASGR that corresponds to a Kd of about 1 pM to about 100 nM, about 1 pM to about 10 nM, or about 1 pm to about 1 nM.

Any appropriate method of determining Kd may be used. However, conveniently the Kd may be determined in a Surface Plasmon Resonance (SPR) assay or system (e.g. a BIAcore assay or system). For example, the Kd may be determined by testing various concentrations of the antigen recognition domain against various concentrations of antigen in vitro to establish a saturation curve, for example using the Lineweaver-Burk method, or by using commercially available binding model software, such as the 1:1 binding model in the BIAcore 1000 Evaluation software. Suitably, the HBS-P buffer system (0.01M Hepes, pH 7.4, 0.15M NaCl, 0.05% surfactant P20) is used. In particular, when said antigen recognition domain is an antibody, an antibody fragment, or derived from an antibody, then the above Kds can be appropriate when the antigen recognition domain is in any format, for example when in scFv or sdAb format, or another format as described elsewhere herein.

The antigen recognition domain may have a melting temperature of 50° C., 55° C., 60° C., 65° C. or greater, preferably 55° C. or greater, for example 55° C. to 75° C. The melting temperature may determined by any method known to those of skill in the art. Suitably the melting temperature is determined by differential scanning calorimetry. Suitably, the antigen recognition domain is heated at 180° C. per hour in 1 mg/mL PBS and a detectable heat change is measure. The transition midpoint gives apparent melting temperature.

The antigen recognition domain (also known as an antigen-specific targeting domain) may be any protein or peptide that possesses the ability to specifically recognise and bind to a liver-specific antigen, preferably asialoglycoprotein receptor (ASGR). The antigen recognition domain includes any naturally occurring, synthetic, semi-synthetic, or recombinantly produced binding partner for a liver-specific antigen, preferably asialoglycoprotein receptor (ASGR). Illustrative antigen recognition domains include antibodies, antibody fragments or derivatives, extracellular domains of receptors, ligands for cell surface molecules/receptors, or receptor binding domains thereof, and tumour binding proteins. For example, suitable ligands for ASGR include beta-D-galactose and N-acetylgalactosamine (GalNAc).

Preferably, the antigen recognition domain is, or is derived from, an antibody (Ab). An antibody-derived antigen recognition domain can be a fragment of an antibody or a genetically engineered product of one of more fragments of the antibody, which fragment is involved in binding with the antigen. Examples include a camelid antibody (VHH), an antigen-binding fragment (Fab), a variable region (Fv), a single chain antibody (scFv), a single-domain antibody (sdAb), a heavy chain variable region (VH), a light chain variable region (VL), and a complementarity determining region (CDR).

In preferred embodiments, the antigen recognition domain is a single chain antibody (scFv) or a single-domain antibody (sdAb). Most preferably, the antigen recognition domain is a single-domain antibody (sdAb).

An antibody recognises an antigen via the fragment antigen-binding (Fab) variable region. Antibodies are glycoproteins belonging to the immunoglobulin superfamily. They constitute most of the gamma globulin fraction of the blood proteins. They are typically made of basic structural units, each with two large heavy chains and two small light chains. Camelid antibodies (VHH) lack light chains, and consist of two heavy chains attached to variable domains.

“Antigen-binding fragment” (Fab) refers to a region on an antibody that binds to antigens. It is composed of one constant and one variable region of each of the heavy and the light chain.

“Fv” refers to the smallest fragment of an antibody to bear the complete antigen binding site. An Fv fragment consists of the variable regions of a single light chain bound to the variable region of a single heavy chain. “Single chain antibody” (scFv) refers to an engineered antibody consisting of a light chain variable region and a heavy chain variable region connected to one another directly or via a peptide linker sequence. The peptide linker sequence is usually about 10 to 25 amino acids in length, rich in glycine for flexibility, and serine or threonine for solubility. The peptide linker sequence can either connect the N-terminus of the heavy chain variable region with the C-terminus of the light chain variable region, or vice versa.

“Single-domain antibody” (sdAb), also known as a nanobody, refers to an antibody fragment consisting of a single monomeric variable antibody domain. Accordingly, a sdAb may be a heavy chain variable region (VH) or a light chain variable region (VL).

“Heavy chain variable region” or “VH” refers to the fragment of the heavy chain of an antibody that contains three CDRs interposed between flanking stretches known as framework regions, which are more highly conserved than the CDRs and form a scaffold to support the CDRs. “Light chain variable region” or “VL” refers to the fragment of the light chain of an antibody that contains three CDRs interposed between framework regions.

“Complementarity determining region” or “CDR” with regard to antibody or antigen-binding fragment thereof refers to a highly variable loop in the variable region of the heavy chain of the light chain of an antibody. CDRs can interact with the antigen conformation and largely determine binding to the antigen (although some framework regions are known to be involved in binding). The heavy chain variable region and the light chain variable region each contain 3 CDRs (heavy chain CDRs 1, 2 and 3 and light chain CDRs 1, 2 and 3, numbered from the amino to the carboxy terminus).

The CDRs of the variable regions of a heavy and light chain of an antibody can be predicted from the heavy and light chain variable region sequences of the antibody, using prediction software available in the art, e.g. using the Abysis algorithm, or using the IMGT/V-QUEST software, e.g. the IMGT algorithm (ImMunoGeneTics) which can be found at www.IMGT.org, (see for example Lefranc et al, 2009 NAR 37:D1006-D1012 and Lefranc 2003, Leukemia 17: 260-266). CDR regions identified by either algorithm are considered to be equally suitable for use in the invention. CDRs may vary in length, depending on the antibody from which they are predicted and between the heavy and light chains. Thus, the three heavy chain CDRs of an intact antibody may be of different lengths (or may be of the same length) and the three light chain CDRs of an intact antibody may be of different lengths (or may be of the same length). A CDR for example, may range from 2 or 3 amino acids in length to 5, 6, 7, 8, 9, 10, 11, 12, 13, 14, 15, 16, 17, 18, 19 or 20 amino acids in length. Particularly, a CDR may be from 3-14 amino acids in length, e.g. at least 3 amino acids and less than 15 amino acids. It should be note that the Kabat nomenclature is followed herein where necessary, in order to define the positioning of the CDRs (Kabat et al, 1991, 5th Ed. Public Health Service, National Institutes of Health, Bethesda, Md., 647-669).

Antibodies, and derivatives and fragments thereof, that specifically bind to a liver-specific antigen, preferably asialoglycoprotein receptor (ASGR), can be prepared using methods well known by those of skill in the art. Such methods include phage display, methods to generate human or humanized antibodies, or methods using transgenic animal or plant engineered to produce human antibodies. Phage display libraries of partially or fully synthetic antibodies are available and can be screened for an antibody or fragment thereof that can bind to the target molecule. Phage display libraries of human antibodies are also available. Once identified, the amino acid sequence or polynucleotide sequence encoding for the antibody (or derivative or fragment thereof) can be isolated and/or determined. The sequence of the antibody can be used to design suitable derivatives or fragments thereof.

Suitable antibodies, and derivatives and fragments thereof, that specifically bind to a liver-specific antigen, preferably asialoglycoprotein receptor (ASGR), are known. For example, anti-NTCP antibodies are described in Stieger, B., et al., 1994. Gastroenterology, 107(6), pp. 1781-1787; anti-mannose-6-phosphate receptor antibodies are described in von Figura, K., et al., 1984. The EMBO journal, 3(6), pp. 1281-1286; anti-PDGF receptor antibodies are described in Ogawa, S., et al., 2010. Hepatology Research, 40(11), pp. 1128-1141; anti-scavenger receptor class A antibodies are described in Tomokiyo, R. I., et al., 2002. Atherosclerosis, 161(1), pp. 123-132; anti scavenger receptor class B type I antibodies are described in Meuleman, P., et al., 2012. Hepatology, 55(2), pp. 364-372; and anti heparan sulfate antibodies are described in Gao, W., et al., 2014. Hepatology, 60(2), pp. 576-587. Antibodies, and derivatives and fragments thereof, which specifically bind to liver-specific antigens are available commercially. The sequence of known antibodies can be used to design suitable derivatives or fragments thereof.

Examples of antibodies, and fragments and derivatives thereof that can be used in the invention are further described below.

The antigen recognition domain may comprise at least one CDR (e.g. CDR3), which can be predicted from an antibody which binds to a liver-specific antigen, preferably asialoglycoprotein receptor (ASGR) (or a variant of such a predicted CDR (e.g. a variant with one, two or three amino acid substitutions)). It will be appreciated that molecules containing three or fewer CDR regions (e.g. a single CDR or even a part thereof) may be capable of retaining the antigen-binding activity of the antibody from which the CDR is derived.

Molecules containing two CDR regions are described in the art as being capable of binding to a target antigen, e.g. in the form of a minibody (Vaughan and Sollazzo, 2001, Combinational Chemistry & High Throughput Screening, 4, 417-430). Molecules containing a single CDR have been described which can display strong binding activity to target (Nicaise et al, 2004, Protein Science, 13: 1882-91).

In this respect, the antigen recognition domain may comprise one or more variable heavy chain CDRs, e.g. one, two or three variable heavy chain CDRs. Alternatively, or additionally, the antigen recognition domain may comprise one or more variable light chain CDRs, e.g. one, two or three variable light chain CDRs. The antigen recognition domain may comprise three heavy chain CDRs and/or three light chain CDRs (and more particularly a heavy chain variable region comprising three CDRs and/or a light chain variable region comprising three CDRs) wherein at least one CDR, preferably all CDRs, may be from an antibody which binds to a liver-specific antigen, preferably ASGR, or may be selected from one of the CDR sequences provided below.

The antigen recognition domain may comprise any combination of variable heavy and light chain CDRs, e.g. one variable heavy chain CDR together with one variable light chain CDR, two variable heavy chain CDRs together with one variable light chain CDR, two variable heavy chain CDRs together with two variable light chain CDRs, three variable heavy chain CDRs together with one or two variable light chain CDRs, one variable heavy chain CDR together with two or three variable light chain CDRs, or three variable heavy chain CDRs together with three variable light chain CDRs. Preferably, the antigen recognition domain comprises three variable heavy chain CDRs (CDR1, CDR2 and CDR3) or three variable light chain CDRs (CDR1, CDR2 and CDR3), i.e. comprises three CDRs. Alternatively, the antigen recognition domain can comprise three variable heavy chain CDRs (CDR1, CDR2 and CDR3) and three variable light chain CDRs (CDR1, CDR2 and CDR3), or otherwise comprise six CDRs.

The one or more CDRs present within the antigen recognition domain may not all be from the same antibody, as long as the domain has the binding activity described above. Thus, one CDR may be predicted from the heavy or light chains of an antibody which binds to a liver-specific antigen, e.g. ASGR whilst another CDR present may be predicted from a different antibody which binds to the same liver-specific antigen (e.g. ASGR). In this instance, it may be preferred that CDR3 be predicted from an antibody that binds to a liver-specific antigen, e.g. ASGR. Particularly however, if more than one CDR is present in the antigen recognition domain, it is preferred that the CDRs are predicted from antibodies which bind to a liver-specific antigen, e.g. ASGR. A combination of CDRs may be used from different antibodies, particularly from antibodies that bind to the same desired region or epitope. Exemplary and preferred CDR sequences are described elsewhere herein.

In a particularly preferred embodiment, the antigen recognition domain comprises three CDRs predicted from the variable heavy chain sequence of an antibody which binds to a liver-specific antigen, e.g. ASGR and/or three CDRs predicted from the variable light chain sequence of an antibody which binds to a liver-specific antigen, e.g. ASGR (preferably the same antibody). Exemplary and preferred CDR sequences are described elsewhere herein.

In some embodiments, the antigen recognition domain is, or is derived from an antibody (e.g. is a Fab, scFv, or sdAb) wherein the antibody comprises one or more CDR regions, selected from SEQ ID NOs: 11-73, or derivatives thereof. In other words, in some embodiments the antigen recognition domain comprises one or more CDR regions, selected from SEQ ID NOs: 11-73, or derivatives thereof. Suitably, the antigen recognition domain comprises three CDR regions selected from SEQ ID NOs: 11-73, or derivatives thereof.

| Name | CDR1 | CDR2 | CDR3 |
|---|---|---|---|
| ASGR1 VH1 CDRs | EKYAMA | RISARGVT | HKRHEHTRFDS |
| (SEQ ID NOs: 11-13) | (SEQ ID NO: 11) | (SEQ ID NO: 12) | (SEQ ID NO: 13) |
| ASGR1 VH2 CDRs | RYTMG | AIGPPGSNTYYA | WVMLRGRFDY |
| (SEQ ID NOs: 14-16) | (SEQ ID NO: 14) | DSVKG | (SEQ ID NO: 16) |
| | | (SEQ ID NO: 15) | |
| ASGR1 VH3 CDRs | DYGMG | AIGRNGSQTYYA | LRRGRGLNTFTLDY |
| (SEQ ID NOs: 17-19) | (SEQ ID NO: 17) | DSVKG | (SEQ ID NO: 19) |
| | | (SEQ ID NO: 18) | |
| ASGR1 VH4 CDRs | AAGMG | AIGRNGSQTYYA | LRRGRGLNTFTLDY |
| (SEQ ID NOs: 20-22) | (SEQ ID NO: 20) | DSVKG (SEQ ID | (SEQ ID NO: 22) |
| | | NO: 21) | |
| ASGR1 VK1 CDRs | RASQAIGRWLL | PGSRLRS | QQAYAWPPT |
| (SEQ ID NOs: 23-25) | (SEQ ID NO: 23) | (SEQ ID NO: 24) | (SEQ ID NO: 25) |
| ASGR1 VK2 CDRs | RASQAIGRWLL | PGSRLQS | QQAYQLPVT |
| (SEQ ID NOs: 26-28) | (SEQ ID NO: 26) | (SEQ ID NO: 27) | (SEQ ID NO: 28) |
| ASGR1 VK3 CDRs | RASQAIGRWLL | PGSRLQS | QQAYSLPPT |
| (SEQ ID NOs: 29-31) | (SEQ ID NO: 29) | (SEQ ID NO: 30) | (SEQ ID NO: 31) |
| ASGR1 VK4 CDRs | RASGDIGHALW | RGGSALQS | GQSHVRPFT |
| (SEQ ID NOs: 32-34) | (SEQ ID NO: 32) | (SEQ ID NO: 33) | (SEQ ID NO: 34) |
| ASGR1 VK5 CDRs | QASKNIGERLV | GFASLLQS | GQYRWVPAT |
| (SEQ ID NOs: 35-37) | (SEQ ID NO: 35) | (SEQ ID NO: 36) | (SEQ ID NO: 37) |
| ASGR1 VH5 CDRs | STYPMH | SISPSGDS | NALRFDY |
| (SEQ ID NOs: 38-40) | (SEQ ID NO: 38) | (SEQ ID NO: 39) | (SEQ ID NO: 40) |
| ASGR1 VH6 CDRs | KPYAMH | SISSTGLS | DASRFRQPFDY |
| (SEQ ID NOs: 41-43) | (SEQ ID NO: 41) | (SEQ ID NO: 42) | (SEQ ID NO: 43) |
| ASGR1 VH7 CDRs | PKYGMA | RIGATGSE | HRGTAHSSFFDY |
| (SEQ ID NOs: 44-46) | (SEQ ID NO: 44) | (SEQ ID NO: 45) | (SEQ ID NO: 46) |
| ASGR1 VH8 CDRs | SANGMH | VISATGDQ | GYDRRHRKFDY |
| (SEQ ID NOs: 47-49) | (SEQ ID NO: 47) | (SEQ ID NO: 48) | (SEQ ID NO: 49) |
| ASGR1 VH9 CDRs | ADYSMY | DISPSGSM | GLPGQNMHVGFDY |
| (SEQ ID NOs: 50-52) | (SEQ ID NO: 50) | (SEQ ID NO: 51) | (SEQ ID NO: 52) |
| ASGR1 VK6 CDRs | RASQAIGRWLL | YAASRLQS | QQAYSLPPT |
| (SEQ ID NOs: 53-55) | (SEQ ID NO: 53) | (SEQ ID NO: 54) | (SEQ ID NO: 55) |
| ASGR1 VK7 CDRs | RASMSIDESLW | RGGSGLQS | GQAARRPYT |
| (SEQ ID NOs: 56-58) | (SEQ ID NO: 56) | (SEQ ID NO: 57) | (SEQ ID NO: 58) |
| ASGR1 VK8 CDRs | RASHYIGNELW | RRGSGLQS | GQARHRPYT |
| (SEQ ID NOs: 59-61) | (SEQ ID NO: 59) | (SEQ ID NO: 60) | (SEQ ID NO: 61) |
| ASGR1 VK9 CDRs | RASSNIGRSLV | AGGSLLQS | GQYAEEPFT |
| (SEQ ID NOs: 62-64) | (SEQ ID NO: 62) | (SEQ ID NO: 63) | (SEQ ID NO: 64) |
| ASGR1 VK10CDRS | RASVKIGERLW | RDASLLQS | GQSWMRPYT |
| (SEQ ID NOs: 65-67) | (SEQ ID NO: 65) | (SEQ ID NO: 66) | (SEQ ID NO: 67) |
| ASGR1 VK11 CDRs | RASSYIGGELW | SGTSGLQS | GQAAKRPFT |
| (SEQ ID NOs: 68-70) | (SEQ ID NO: 68) | (SEQ ID NO: 69) | (SEQ ID NO: 70) |
| ASGR1 VK12 CDRs | RASSWINSDLV | AGGSLLQS | GQYLEEPYT |
| (SEQ ID NOs: 71-73) | (SEQ ID NO: 71) | (SEQ ID NO: 72) | (SEQ ID NO: 73) |

Preferably, the antigen binding domain comprises CDRs (CDR1, CDR2, and CDR3), or derivatives thereof, selected from the same variable chain. For example, the antigen binding domain may comprise SEQ ID NOs: 11-13, SEQ ID NOs: 14-16, SEQ ID NOs: 17-19, SEQ ID NOs: 20-22, SEQ ID NOs: 23-25, SEQ ID NOs: 26-28, SEQ ID NOs: 29-31, SEQ ID NOs: 32-34, SEQ ID NOs: 35-37, SEQ ID NOs: 38-40, SEQ ID NOs: 41-43, SEQ ID NOs: 44-46, SEQ ID NOs: 47-49, SEQ ID NOs: 50-52, SEQ ID NOs: 53-55, SEQ ID NOs: 56-58, SEQ ID NOs: 59-61, SEQ ID NOs: 62-64, SEQ ID NOs: 65-67, SEQ ID NOs: 68-70, and/or SEQ ID NOs: 71-73, or derivatives thereof.

Preferably, the antigen binding domain may comprise SEQ ID NOs: 11-13, SEQ ID NOs: 14-16, SEQ ID NOs: 17-19, SEQ ID NOs: 20-22, SEQ ID NOs: 23-25, SEQ ID NOs: 26-28, and/or SEQ ID NOs: 29-31, or derivatives thereof.

Preferably, the antigen binding domain may comprise SEQ ID NOs: 11-13, SEQ ID NOs: 14-16, SEQ ID NOs: 17-19, SEQ ID NOs: 23-25, SEQ ID NOs: 26-28, and/or SEQ ID NOs: 29-31, or derivatives thereof.

Preferably, the antigen binding domain may comprise SEQ ID NOs: 11-13, SEQ ID NOs: 14-16, or SEQ ID NOs: 17-19, and/or SEQ ID NOs: 23-25, SEQ ID NOs: 26-28, or SEQ ID NOs: 29-31, or derivatives thereof.

In preferred embodiments, the antigen recognition domain comprises one or more CDR regions selected from SEQ ID NOs: 11-31. Suitably, the antigen recognition domain comprises three CDR regions selected from SEQ ID NOs: 11-31, or derivatives thereof.

In preferred embodiments, the antigen recognition domain comprises:
(i) CDR1 sequence EKYAMA (SEQ ID NO: 11), CDR2 sequence RISARGVT (SEQ ID NO: 12), and CDR3 sequence HKRHEHTRFDS (SEQ ID NO: 13), or derivatives thereof;
(ii) CDR1 sequence RYTMG (SEQ ID NO: 14), CDR2 sequence AIGPPGSNTYYADSVKG (SEQ ID NO: 15), and CDR3 sequence WVMLRGRFDY (SEQ ID NO: 16), or derivatives thereof;
(iii) CDR1 sequence DYGMG (SEQ ID NO: 17), CDR2 sequence AIGRNGSQTYYADSVKG (SEQ ID NO: 18), and CDR3 sequence LRRGRGLNTFTLDY (SEQ ID NO: 19), or derivatives thereof;
(iv) CDR1 sequence AAGMG (SEQ ID NO: 20), CDR2 sequence AIGRNGSQTYYADSVKG (SEQ ID NO: 21), and CDR3 sequence LRRGRGLNTFTLDY (SEQ ID NO: 22), or derivatives thereof;
(v) CDR1 sequence RASQAIGRWLL (SEQ ID NO: 23), CDR2 sequence PGSRLRS (SEQ ID NO: 24), and CDR3 sequence QQAYAWPPT (SEQ ID NO: 25), or derivatives thereof;
(vi) CDR1 sequence RASQAIGRWLL (SEQ ID NO: 26), CDR2 sequence PGSRLQS (SEQ ID NO: 27), and CDR3 sequence QQAYQLPVT (SEQ ID NO: 28), or derivatives thereof; and/or
(vii) CDR1 sequence RASQAIGRWLL (SEQ ID NO: 29), CDR2 sequence PGSRLQS (SEQ ID NO: 30), and CDR3 sequence QQAYSLPPT (SEQ ID NO: 31), or derivatives thereof.

Most preferably, the antigen recognition domain comprises CDR1 sequence EKYAMA (SEQ ID NO: 11), CDR2 sequence RISARGVT (SEQ ID NO: 12), and CDR3 sequence HKRHEHTRFDS (SEQ ID NO: 13), or derivatives thereof. Preferably, the antigen recognition domain is a sdAb comprising CDR1 sequence EKYAMA (SEQ ID NO: 11), CDR2 sequence RISARGVT (SEQ ID NO: 12), and CDR3 sequence HKRHEHTRFDS (SEQ ID NO: 13), or derivatives thereof.

In other embodiments, the antigen recognition domain comprises CDR1, CDR2 and CDR3 regions comprising:

(i) SEQ ID NOs: 11, 12 and 13, respectively, or derivatives thereof; and SEQ ID NOs: 23, 24 and 25, respectively, or derivatives thereof; or SEQ ID NOs: 26, 27 and 28, respectively, or derivatives thereof; or SEQ ID NOs: 29, 30 and 31, respectively, or derivatives thereof; preferably wherein the antigen recognition domain comprises CDR1, CDR2 and CDR3 regions comprising SEQ ID NOs: 11, 12 and 13, respectively; and SEQ ID NOs: 23, 24 and 25, respectively;

(ii) SEQ ID NOs: 14, 15 and 16, respectively, or derivatives thereof; and SEQ ID NOs: 23, 24 and 25, respectively, or derivatives thereof; or SEQ ID NOs: 26, 27 and 28, respectively, or derivatives thereof; or SEQ ID NOs: 29, 30 and 31, respectively, or derivatives thereof; preferably wherein the antigen recognition domain comprises CDR1, CDR2 and CDR3 regions comprising SEQ ID NOs: 14, 15 and 16, respectively; and SEQ ID NOs: 26, 27 and 28, respectively; or

(iii) SEQ ID NOs: 17, 18 and 19, respectively, or derivatives thereof; and SEQ ID NOs: 23, 24 and 25, respectively, or derivatives thereof; or SEQ ID NOs: 26, 27 and 28, respectively, or derivatives thereof; or SEQ ID NOs: 29, 30 and 31, respectively, or derivatives thereof; preferably wherein the antigen recognition domain comprises CDR1, CDR2 and CDR3 regions comprising SEQ ID NOs: 17, 18 and 19, respectively; and SEQ ID NOs: 29, 30 and 31, respectively.

Thus, in such embodiments, the antigen recognition domain may comprise CDR1, CDR2 and CDR3 regions comprising SEQ ID NOs: 11, 12 and 13, respectively, or derivatives thereof; and SEQ ID NOs: 23, 24 and 25, respectively, or derivatives thereof.

The antigen recognition domain may comprise CDR1, CDR2 and CDR3 regions comprising SEQ ID NOs: 11, 12 and 13, respectively, or derivatives thereof; and SEQ ID NOs: 26, 27 and 28, respectively, or derivatives thereof.

The antigen recognition domain may comprise CDR1, CDR2 and CDR3 regions comprising SEQ ID NOs: 11, 12 and 13, respectively, or derivatives thereof; and SEQ ID NOs: 29, 30 and 31, respectively, or derivatives thereof.

The antigen recognition domain may comprise CDR1, CDR2 and CDR3 regions comprising SEQ ID NOs: 14, 15 and 16, respectively, or derivatives thereof; and SEQ ID NOs: 23, 24 and 25, respectively, or derivatives thereof.

The antigen recognition domain may comprise CDR1, CDR2 and CDR3 regions comprising SEQ ID NOs: 14, 15 and 16, respectively, or derivatives thereof; and SEQ ID NOs: 26, 27 and 28, respectively, or derivatives thereof.

The antigen recognition domain may comprise CDR1, CDR2 and CDR3 regions comprising SEQ ID NOs: 14, 15 and 16, respectively, or derivatives thereof; and SEQ ID NOs: 29, 30 and 31, respectively, or derivatives thereof.

The antigen recognition domain may comprise CDR1, CDR2 and CDR3 regions comprising SEQ ID NOs: 17, 18 and 19, respectively, or derivatives thereof; and SEQ ID NOs: 23, 24 and 25, respectively, or derivatives thereof.

The antigen recognition domain may comprise CDR1, CDR2 and CDR3 regions comprising SEQ ID NOs: 17, 18 and 19, respectively, or derivatives thereof; and SEQ ID NOs: 26, 27 and 28, respectively, or derivatives thereof.

The antigen recognition domain may comprise CDR1, CDR2 and CDR3 regions comprising SEQ ID NOs: 17, 18 and 19, respectively, or derivatives thereof; and SEQ ID NOs: 29, 30 and 31, respectively, or derivatives thereof.

CARs comprising such antigen recognition domains form a yet further aspect of the invention.

Preferably the above antigen recognition domains comprising six CDRs are scFvs.

The present invention includes “derivatives” of the CDR regions (and VH and VL (VK) regions, and CDR regions within said regions) described above and elsewhere herein. The term “derivative” as used herein is defined below in the section “Variants, derivatives and fragments”. It will be appreciated that one or more amino acid substitutions may be made in the CDRs whilst retaining the antigen-binding ability. For instance, the CDR derivatives may comprise 3 or fewer amino acid substitutions, e.g. 3 amino acid substitutions, 2 amino acid substitutions or 1 amino acid substitution. In particular, in some embodiments the CDR derivatives comprise one amino acid substitution and retain the antigen-binding ability. The derivative may be a variant, for example a variant with at least 80% or 90% identity to the CDR.

In some embodiments, the antigen recognition domain comprises or consists of an amino acid sequence which has at least about 70%, 80%, 85%, 90%, 95%, 98%, 99%, or 100% identity to one or more of SEQ ID NOs: 74-94.

| Name | Sequence |
|---|---|
| ASGR1 VH1 | EVQLLESGGGLVQPGGSLRLSCAASGFTFEKYAMAWVRQAPGKGLEW |
| (SEQ ID NO: 74) | VSRISARGVTTYYADSVKGRFTISRDNSKNTLYLQMNSLRAEDTAVYYC |
| | AKHKRHEHTRFDSWGQGTLVTVSS |
| ASGR1 VH2 | ELQLLEFGGGLVQPGGSLRLSCTTSGFTFSRYTMGWVRQAPGKGLEW |
| (SEQ ID NO: 75) | VSAIGPPGSNTYYADSVKGRFTISRDNSKNTLYLQMNSLRAEDTAVYYC |
| | AKWVMLRGRFDYWGQGTLVTVSS |
| ASGR1 VH3 | EVQLLESGGGLVQPGGSLRLSCAASGFTFEDYGMGWVRQAPGKGLE |
| (SEQ ID NO: 76) | WVSAIGRNGSQTYYADSVKGRFTISRDNSKNTLYLQMNSLRAEDTAVY |
| | YCAKLRRGRGLNTFTLDYWGQGTLVTVSS |
| ASGR1 VH4 | EVQLLESGGGLVQPGGSLRLSCAASGFTFGAAGMGWVRQAPGKGLE |
| (SEQ ID NO: 77) | WVSAIGRNGSQTYYADSVKGRFTISRDNSKNTLYLQMNSLRAEDTAVY |
| | YCAKLRRGRGLNTFTLDYWGQGTLVTVSS |
| ASGR1 VK1 | DIQMTQSPSSLSASVGDRVTITCRASQAIGRWLLWYQQKPGKAPKLLIG |
| (SEQ ID NO: 78) | PGSRLRSGVPSRFSGSGSGTDFTLTISSLQPEDFVTYYCQQAYAWPPT |
| | FGQGTKVEIKR |
| ASGR1 VK2 | DIQMTQSPSSLSASVGDRVTITCRASQAIGRWLLWYQQKPGKAPKHLIG |
| (SEQ ID NO: 79) | PGSRLQSGVPSRFSGSGSGTDFTLTISSLQPEDFATYYCQQAYQLPVTF |
| | GQGTKVEIKR |
| ASGR1 VK3 | DIQMTQSPSSLSASVGDRVTITCRASQAIGRWLLWYQQKPGKAPKLLIG |
| (SEQ ID NO: 80) | PGSRLQSGVPSRFSGSGSGTDFTLTIGSLQPEDFATYYCQQAYSLPPTF |
| | GQGTKVEIKR |
| ASGR1 VK4 | DIQMTQSPSSLSASVGDRVTITCRASGDIGHALWWYQQKPGKAPKLLIR |
| (SEQ ID NO: 81) | GGSALQSGVPSRFSGSGSGTDFTLTISSLQPEDFATYYCGQSHVRPFTF |
| | GQGTKVEIKR |
| ASGR1 VK5 | DIQMTQSPSSLSASVGDRVTITCQASKNIGERLVWYQQKPGKAPKLLIG |
| (SEQ ID NO: 82) | FASLLQSGVPSRFSGSGSGTDFTLTISSLQPEDFATYYCGQYRWVPATF |
| | GQGTKVEIKR |
| ASGR1 VH5 | EVQLLESGGGLVQPGGSLRLSCAASGFTFSTYPMHWARQAPGKGLEW |
| (SEQ ID NO: 83) | VSSISPSGDSTYYADSVKGRFTISRDNSKNTLYLQMNSLRAEDTAVYYC |
| | AKNALRFDYWGQGTLVTVSS |
| ASGR1 VH6 | EVQLLESGGGLVQPGGSLRLSCAASGFTFKPYAMHWVRQAPGKGLEW |
| (SEQ ID NO: 84) | VSSISSTGLSTYYADSVKGRFTISRDNSKNTLYLQMNSLRAEDTAVYYCA |
| | KDASRFRQPFDYWGQGTLVTVSS |
| ASGR1 VH7 | EVQLLESGGGLVQPGGSLRLSCAASGFTFPKYGMAWVRQAPGKGLEW |
| (SEQ ID NO: 85) | VSRIGATGSETYYADSVKGRFTISRDNSKNTLYLQMNSLRAEDTAVYYC |
| | AKHRGTAHSSFFDYWGQGTLVTVSS |
| ASGR1 VH8 | EVQLLESGGGLVQPGGSLRLSCAASGFTFSANGMHWVRQAPGKGLEW |
| (SEQ ID NO: 86) | VSVISATGDQTYYADSVKGRFTISRDNSKNTLYLQMNSLRAEDTAVYYC |
| | AKGYDRRHRKFDYWGQGTLVTVSS |
| ASGR1 VH9 | EVQLLESGGGLVQPGGSLRLSCAASGFTFADYSMYWVRQAPGKGLEW |
| (SEQ ID NO: 87) | VSDISPSGSMTYYADSVKGRFTISRDNSKNTLYLQMNSLRAEDTAVYYC |
| | AKGLPGQNMHVGFDYWGQGTLVTVSS |
| ASGR1 VK6 | DIQMTQSPSSLSASVGDRVTITCRASQAIGRWLLWYQQKPGKAPKLLIY |
| (SEQ ID NO: 88) | AASRLQSGVPSRFSGSGSGTDFTLTISSLQPEDFATYYCQQAYSLPPTF |
| | GQGTKVEIKR |
| ASGR1 VK7 | DIQMTQSPSSLSASVGDRVTITCRASMSIDESLWWYQQKPGKAPKLLIR |
| (SEQ ID NO: 89) | GGSGLQSGVPSRFSGSGSGTDFTLTISSLQPEDFATYYCGQAARRPYT |
| | FGQGTKVEIKR |
| ASGR1 VK8 | DIQMTQSPSSLSASVGDRVTITCRASHYIGNELWWYQQKPGKAPKLLIR |
| (SEQ ID NO: 90) | RGSGLQSGVPSRFSGSGSGTDFTLTISSLQPEDFATYYCGQARHRPYT |
| | FGQGTRVEIKR |
| ASGR1 VK9 | DIQMTQSPSSLSASVGDRVTITCRASSNIGRSLVWYQQKPGKAPKLLIA |
| (SEQ ID NO: 91) | GGSLLQSGVPSRFSGSGSGTDFTLTISSLQPEDFATYYCGQYAEEPFTF |
| | GQGTRVEIKR |
| ASGR1 VK10 | DIQMTQSPSSLSASVGDRVTITCRASVKIGERLWWYQQKPGEAPKLLIR |
| (SEQ ID NO: 92) | DASLLQSGVPSRFSGSGSGTDFTLTISSLQPEDFATYYCGQSWMRPYT |
| | FGQGTKVEIKR |
| ASGR1 VK11 | DIQMTQSPSSLSASVGDRVTITCRASSYIGGELWWYQQKPGKAPKLLIS |
| (SEQ ID NO: 93) | GTSGLQSGVPSRFSGSGSGTDFTLTISSLQPEDFATYYCGQAAKRPFTF |
| | GQGTKVEIKR |
| ASGR1 VK12 | DIQMTQSPSSLSASVGDRVTITCRASSWINSDLVWYQQKPGEAPKLLIA |
| (SEQ ID NO: 94) | GGSLLQSGVPSRFSGSGSGTDFTLTISSLQPEDFATYYCGQYLEEPYTF |
| | GQGTKVEVKR |

In preferred embodiments, the antigen recognition domain comprises or consists of an amino acid sequence which has at least about 70%, 80%, 85%, 90%, 95%, 98%, 99%, or 100% identity to one or more of SEQ ID NOs: 74-80.

Most preferably, the antigen recognition domain comprises or consists of an amino acid sequence which has at least about 70%, 80%, 85%, 90%, 95%, 98%, 99%, or 100% identity to ASGR1 VH1 (SEQ ID NO: 74).

In some embodiments, the antigen recognition domain comprises or consists of an amino acid sequence of SEQ ID NOs: 74, 75 or 76, or derivatives thereof, and/or an amino acid sequence of SEQ ID NOs: 78, 79 or 80, or derivatives thereof. In preferred embodiments, the antigen recognition domain comprises or consists of amino acid sequences of SEQ ID NOs: 74 and 78, or derivatives thereof; SEQ ID NOs: 75 and 79, or derivatives thereof; or SEQ ID NOs: 76 and 80, or derivatives thereof.

Thus, in such embodiments, the antigen recognition domain may comprise or consist of SEQ ID NOs: 74 and 78, or derivatives thereof. The antigen recognition domain may comprise or consist of SEQ ID NOs: 74 and 79, or derivatives thereof. The antigen recognition domain may comprise or consist of SEQ ID NOs: 74 and 80, or derivatives thereof.

The antigen recognition domain may comprise or consist of SEQ ID NOs: 75 and 78, or derivatives thereof. The antigen recognition domain may comprise or consist of SEQ ID NOs: 75 and 79, or derivatives thereof. The antigen recognition domain may comprise or consist of SEQ ID NOs: 75 and 80, or derivatives thereof.

The antigen recognition domain may comprise or consist of SEQ ID NOs: 76 and 78, or derivatives thereof. The antigen recognition domain may comprise or consist of SEQ ID NOs: 76 and 79, or derivatives thereof. The antigen recognition domain may comprise or consist of SEQ ID NOs: 76 and 80, or derivatives thereof.

Preferably the above antigen recognition domains comprising a VH and VL (VK) domain are scFvs. Such scFvs can contain a linker between the VH and VL (VK) domains.

The antigen recognition domain may further comprise SEQ ID NO. 74 or a derivative thereof and any one of SEQ ID Nos 81, 82 or 88 to 94 or derivatives thereof.

The antigen recognition domain may further comprise SEQ ID NO. 75 or a derivative thereof and any one of SEQ ID Nos 81, 82 or 88 to 94 or derivatives thereof.

The antigen recognition domain may further comprise SEQ ID NO. 76 or a derivative thereof and any one of SEQ ID Nos 81, 82 or 88 to 94 or derivatives thereof.

The antigen recognition domain may further comprise SEQ ID NO. 77 or a derivative thereof and any one of SEQ ID Nos 78 to 82 or 88 to 94 or derivatives thereof.

The antigen recognition domain may further comprise SEQ ID NO. 83 or a derivative thereof and any one of SEQ ID Nos 78 to 82 or 88 to 94 or derivatives thereof.

The antigen recognition domain may further comprise SEQ ID NO. 84 or a derivative thereof and any one of SEQ ID Nos 78 to 82 or 88 to 94 or derivatives thereof.

The antigen recognition domain may further comprise SEQ ID NO. 85 or a derivative thereof and any one of SEQ ID Nos 78 to 82 or 88 to 94 or derivatives thereof.

The antigen recognition domain may further comprise SEQ ID NO. 86 or a derivative thereof and any one of SEQ ID Nos 78 to 82 or 88 to 94 or derivatives thereof.

The antigen recognition domain may further comprise SEQ ID NO. 87 or a derivative thereof and any one of SEQ ID Nos 78 to 82 or 88 to 94 or derivatives thereof.

Some exemplary scFvs (scFv fragments) are outlined below:

(SEQ ID NO: 173)
EVQLLESGGGLVQPGGSLRLSCAASGFTFEKYAMAWVRQAPGKGLEWVSRISARGVTTYYADSVKGRF
TISRDNSKNTLYLQMNSLRAEDTAVYYCAKHKRHEHTRFDSWGQGTLVTVSSLVTVSSGGGGSGGGGS
GGGGSDIQMTQSPSSLSASVGDRVTITCRASQAIGRWLLWYQQKPGKAPKLLIGPGSRLRSGVPSRFS
GSGSGTDFTLTISSLQPEDFVTYYCQQAYAWPPTFGQGTKVEIKR
(SEQ ID NO: 174)
ELQLLEFGGGLVQPGGSLRLSCTTSGFTFSRYTMGWVRQAPGKGLEWVSAIGPPGSNTYYADSVKGRF
TISRDNSKNTLYLQMNSLRAEDTAVYYCAKWVMLRGRFDYWGQGTLVTVSSLVTVSSGGGGSGGGGSG
GGGSDIQMTQSPSSLSASVGDRVTITCRASQAIGRWLLWYQQKPGKAPKHLIGPGSRLQSGVPSRFSG
SGSGTDFTLTISSLQPEDFATYYCQQAYQLPVTFGQGTKVEIKR
(SEQ ID NO: 175)
EVQLLESGGGLVQPGGSLRLSCAASGFTFEDYGMGWVRQAPGKGLEWVSAIGRNGSQTYYADSVKGRF
TISRDNSKNTLYLQMNSLRAEDTAVYYCAKLRRGRGLNTFTLDYWGQGTLVTVSSLVTVSSGGGGSGG
GGSGGGGSDIQMTQSPSSLSASVGDRVTITCRASQAIGRWLLWYQQKPGKAPKLLIGPGSRLQSGVPS
RFSGSGSGTDFTLTIGSLQPEDFATYYCQQAYSLPPTFGQGTKVEIKR

CARs comprising such antigen recognition domains form a yet further aspect of the invention.

The antigen recognition domain variants described herein retain antigen-binding ability. For example, the variants may be capable of binding ASGR to at least 10%, at least 20%, at least 30%, at least 40%, at least 50%, at least 60%, at least 70%, at least 80%, or at least 90% of the level of the corresponding reference amino acid sequence. The variant may be capable of binding ASGR to a similar or the same level as the corresponding reference amino acid sequence or may be capable of binding ASGR to a greater level than the corresponding reference amino acid sequence (e.g. increased by at least 10%, at least 20%, at least 30%, at least 40% or at least 50%). Accordingly, the antigen recognition domain may comprise or consist of an amino acid sequence comprising the CDRs of ASGR1 VH1-4 (SEQ ID NOs: 11-22) and/or ASGR1 VK1-3 (SEQ ID NOs: 23-31) (shown above, underlined in SEQ ID NOs: 74-77 and SEQ ID NOs: 78-80, respectively). The antigen recognition domain may comprise or consist of an amino acid sequence comprising the CDRs of ASGR1 VH1-3 (SEQ ID NOs: 11-19) and/or ASGR1 VK1-3 (SEQ ID NOs: 23-31) (shown above, underlined in SEQ ID NOs: 74-76 and SEQ ID NOs: 78-80, respectively). Substitutions, variations, modifications, replacements, deletions and/or additions of one (or more) amino acid residues may occur in the framework region.

Thus, in some embodiments the antigen recognition domain comprises or consists of:
(i) an amino acid sequence which has at least about 70%, 80%, 85%, 90%, 95%, 98%, 99%, or 100% identity to ASGR1 VH1 (SEQ ID NO: 74), wherein the amino acid sequence comprises CDR1 sequence EKYAMA (SEQ ID NO: 11), CDR2 sequence RISARGVT (SEQ ID NO: 12), and CDR3 sequence HKRHEHTRFDS (SEQ ID NO: 13);
(ii) an amino acid sequence which has at least about 70%, 80%, 85%, 90%, 95%, 98%, 99%, or 100% identity to ASGR1 VH2 (SEQ ID NO: 75), wherein the amino acid sequence comprises CDR1 sequence RYTMG (SEQ ID NO: 14), CDR2 sequence AIGPPGSNTYYADSVKG (SEQ ID NO: 15), and CDR3 sequence WVMLRGRFDY (SEQ ID NO: 16);
(iii) an amino acid sequence which has at least about 70%, 80%, 85%, 90%, 95%, 98%, 99%, or 100% identity to ASGR1 VH3 (SEQ ID NO: 76), wherein the amino acid sequence comprises CDR1 sequence DYGMG (SEQ ID NO: 17), CDR2 sequence AIGRNGSQTYYADSVKG (SEQ ID NO: 18), and CDR3 sequence LRRGRGLNTFTLDY (SEQ ID NO: 19);
(iv) an amino acid sequence which has at least about 70%, 80%, 85%, 90%, 95%, 98%, 99%, or 100% identity to ASGR1 VH4 (SEQ ID NO: 77), wherein the amino acid sequence comprises CDR1 sequence AAGMG (SEQ ID NO: 20), CDR2 sequence AIGRNGSQTYYADSVKG (SEQ ID NO: 21), and CDR3 sequence LRRGRGLNTFTLDY (SEQ ID NO: 22);
(v) an amino acid sequence which has at least about 70%, 80%, 85%, 90%, 95%, 98%, 99%, or 100% identity to ASGR1 VK1 (SEQ ID NO: 78), wherein the amino acid sequence comprises CDR1 sequence RASQAIGRWLL (SEQ ID NO: 23), CDR2 sequence PGSRLRS (SEQ ID NO: 24), and CDR3 sequence QQAYAWPPT (SEQ ID NO: 25);
(vi) an amino acid sequence which has at least about 70%, 80%, 85%, 90%, 95%, 98%, 99%, or 100% identity to ASGR1 VK2 (SEQ ID NO: 79), wherein the amino acid sequence comprises CDR1 sequence RASQAIGRWLL (SEQ ID NO: 26), CDR2 sequence PGSRLQS (SEQ ID NO: 27), and CDR3 sequence QQAYQLPVT (SEQ ID NO: 28); or
(vii) an amino acid sequence which has at least about 70%, 80%, 85%, 90%, 95%, 98%, 99%, or 100% identity to ASGR1 VK3 (SEQ ID NO: 80), wherein the amino acid sequence comprises CDR1 sequence RASQAIGRWLL (SEQ ID NO: 29), CDR2 sequence PGSRLQS (SEQ ID NO: 30), and CDR3 sequence QQAYSLPPT (SEQ ID NO: 31).

In a preferred embodiment the antigen recognition domain comprises or consists of an amino acid sequence which has at least about 70%, 80%, 85%, 90%, 95%, 98%, 99%, or 100% identity to ASGR1 VH1 (SEQ ID NO: 74), wherein the amino acid sequence comprises CDR1 sequence EKYAMA (SEQ ID NO: 11), CDR2 sequence RISARGVT (SEQ ID NO: 12), and CDR3 sequence HKRHEHTRFDS (SEQ ID NO: 13), or derivatives thereof.

Although a CAR of the invention may comprise more than one antigen recognition domain, i.e. may bind to more than one of the liver specific antigens, or more than one epitope within a liver specific antigen as defined above, for example, as part of a dual or two polypeptide CAR system, in a particular embodiment, the CAR of the invention may comprise only one or a single antigen recognition domain.

Hinge Domain

The CAR may comprise a hinge domain.

The “hinge domain”, also referred to as the “spacer domain”, as used herein refers to the extracellular part of the CAR that separates the antigen binding domain from the transmembrane domain. The hinge may provide flexibility to access the targeted antigen. For example, long spacers provide extra flexibility to the CAR and allow for better access to membrane-proximal epitopes

Suitable hinge domains will be apparent to those of skill in the art (e.g. Guedan, S., et al., 2018. Molecular Therapy-Methods & Clinical Development, 12, 145-156). Suitable hinge domains include, but are not limited to: CD28 hinge domain, a CD8α hinge domain, an IgG hinge domain, and an IgD hinge domain. Preferably the hinge domain is a CD8α or CD28 hinge domain.

Suitably, the hinge domain may comprise the amino acid sequence shown as SEQ ID NO: 95, or a variant which is at least 80% identical to SEQ ID NO: 95.

Illustrative CD28 hinge domain (SEQ ID NO: 95):
IEVMYPPPYLDNEKSNGTIIHVKGKHLCPSPLFPGPSKP

Suitably, the variant may be at least 85, 90, 95, 97, 98 or 99% identical to SEQ ID NO: 95.

Suitably, the hinge domain may comprise the amino acid sequence shown as SEQ ID NO: 96, or a variant which is at least 80% identical to SEQ ID NO: 96.

Illustrative CD8 alpha hinge domain (SEQ ID
NO: 96):
TTTPAPRPPTPAPTIASQPLSLRPEACRPAAGGAVHTRGLDFACD

Suitably, the variant may be at least 85, 90, 95, 97, 98 or 99% identical to SEQ ID NO: 96.

Suitably, the CAR may encode a tag, such as a c-Myc tag (EQKLISEEDL—SEQ ID NO: 97). Suitably the tag may be incorporated into the extracellular domain of the CAR, for example in the hinge domain of the extracellular domain. An illustrative CD28 hinge domain with an integrated c-Myc tag is shown below.

Illustrative CD28 hinge domain with an integrated
c-Myc tag (SEQ ID NO: 98):
IEVEQKLISEEDLLDNEKSNGTIIHVKGKHLCPSPLFPGPSKP

Suitably, the variant may be at least 85, 90, 95, 97, 98 or 99% identical to SEQ ID NO: 98.

Transmembrane Domain

The CAR may comprise a transmembrane domain.

The “transmembrane domain” as used herein refers to the part of the CAR that anchors the CAR into the cell membrane of the Treg. Thus, the transmembrane domain is capable of spanning or being present within the cell membrane of the Treg. The transmembrane domain may be derived from a protein comprising an extracellular and/or intracellular portions and thus the transmembrane domain as used herein may be attached to extracellular and/or intracellular residues derived from the protein of origin, in addition to the portion within or spanning the cell membrane. For example, the transmembrane domain may be attached to a hinge domain derived from the protein of origin e.g. a transmembrane domain derived from CD8α may be attached to a hinge domain derived from CD8α. The presence of a transmembrane domain within a cell membrane can be assessed using any suitable method known in the art, including fluorescence labelling with fluorescence microscopy.

Suitable transmembrane domains will be apparent to those of skill in the art. The transmembrane domain may comprise the transmembrane sequence from any protein which has a transmembrane domain, including any of the type I, type II or type III transmembrane proteins. The transmembrane domain of the CAR may also comprise an artificial hydrophobic sequence. The transmembrane domain may be selected so as not to dimerize.

Examples of transmembrane (TM) domains used in CAR constructs are: 1) The CD28 TM domain (Pule et al, Mol Ther, 2005, November; 12(5):933-41; Brentjens et al, CCR, 2007, Sep. 15; 13(18 Pt 1):5426-35; Casucci et al, Blood, 2013, Nov. 14; 122(20):3461-72.); 2) The OX40 TM domain (Pule et al, Mol Ther, 2005, November; 12(5):933-41); 3) The 41BB TM domain (Brentjens et al, CCR, 2007, Sep. 15; 13(18 Pt 1):5426-35); 4) The CD3 zeta TM domain (Pule et al, Mol Ther, 2005, November; 12(5):933-41; Savoldo B, Blood, 2009, Jun. 18; 113(25):6392-402.); 5) The CD8 alpha TM domain (Maher et al, Nat Biotechnol, 2002, January; 20(1):70-5.; Imai C, Leukemia, 2004, April; 18(4):676-84; Brentjens et al, CCR, 2007, Sep. 15; 13(18 Pt 1):5426-35; Milone et al, Mol Ther, 2009, August; 17(8):1453-64.); 6) the ICOS TM domain; 7) the CD4 TM domain.

Suitably, the CAR may comprise the CD28 transmembrane domain. Suitably, the transmembrane domain may comprise the amino acid sequence shown as SEQ ID NO: 99, or a variant which is at least 80% identical to SEQ ID NO: 99.

Illustrative CD28 TM domain (AA 153 to 179) (SEQ
ID NO: 99):
FWWVLVVVGGVLACYSLLVTVAFIFWV

Suitably, the variant may be at least 85, 90, 95, 97, 98 or 99% identical to SEQ ID NO: 99.

Suitably, the CAR may comprise the CD8α transmembrane domain. Suitably, the transmembrane domain may comprise the amino acid sequence shown as SEQ ID NO: 165, or a variant which is at least 80% identical to SEQ ID NO: 165.

Illustrative CD8α TM domain (AA 183 to 203) (SEQ
ID NO: 165):
IYIWAPLAGTCGVLLLSLVIT

Suitably, the variant may be at least 85, 90, 95, 97, 98 or 99% identical to SEQ ID NO: 165.

Suitably, the CAR may comprise the CD28 hinge and transmembrane domain (optionally with c-Myc tag). Suitably, the hinge and transmembrane domain may comprise the amino acid sequence shown as SEQ ID NO: 100 or SEQ ID NO: 101, or a variant which is at least 80% identical to SEQ ID NO: 100 or SEQ ID NO: 101.

Illustrative CD28 hinge and transmembrane domain
(SEQ ID NO: 100):
IEVMYPPPYLDNEKSNGTIIHVKGKHLCPSPLFPGPSKPFWVLVWGGVL
ACYSLLVTVAFIIFWV
Illustrative CD28 hinge and transmembrane domain
with c-Myc tag (SEQ ID NO: 101):
IEVEQKLISEEDLLDNEKSNGTIIHVKGKHLCPSPLFPGPSKPFWVLVW
GGVLACYSLLVTVAFIIFWV

Suitably, the variant may be at least 85, 90, 95, 97, 98 or 99% identical to SEQ ID NO: 100 or SEQ ID NO: 101.

Suitably, the CAR may comprise the CD8α hinge domain and the CD28 transmembrane domain. Suitably, the hinge and transmembrane domain may comprise the amino acid sequence shown as SEQ ID NO: 102, or a variant which is at least 80% identical to SEQ ID NO: 102.

Illustrative CD8α hinge domain and the CD28
transmembrane domain (SEQ ID NO: 102):
TTTPAPRPPTPAPTIASQPLSLRPEACRPAAGGAVHTRGLDFACDFWLV
VVGGVLACYSLLVTVAFIIFWV

Suitably, the variant may be at least 85, 90, 95, 97, 98 or 99% identical to SEQ ID NO: 102.

Suitably, the CAR may comprise the CD28 hinge domain and the CD8α transmembrane domain. Suitably, the hinge and transmembrane domain may comprise the amino acid sequence shown as SEQ ID NO: 166, or a variant which is at least 80% identical to SEQ ID NO: 166.

Illustrative CD28 hinge domain and the CD8α
transmembrane domain (SEQ ID NO: 166):
IEVMYPPPYLDNEKSNGTIIHVKGKHLCPSPLFPGPSKPIYIWAPLAGT
CGVLLLSLVIT

Suitably, the variant may be at least 85, 90, 95, 97, 98 or 99% identical to SEQ ID NO: 166.

Endodomain

The CAR may comprise an endodomain comprising one or more intracellular signalling domains and optionally one or more co-stimulatory domains.

The CAR may comprise one or more intracellular signalling domains.

The “intracellular signalling domain” as used herein refers to the intracellular part of the CAR that participates in transducing the message of the effective CAR binding to the liver-specific antigen (e.g. ASGR) into the interior of the Treg to elicit Treg function, e.g. immunosuppressive function.

Suitable intracellular signalling domains will be apparent to those of skill in the art. The intracellular signalling domain is necessary to transduce the effector function signal and direct the Treg to perform its specialized function upon antigen binding. Examples of intracellular signalling domains include, but are not limited to, ζ chain of the T-cell receptor or any of its homologs (e.g., η chain, FcεR1γ and β chains, MB1 (Igα) chain, B29 (Igβ) chain, etc.), CD3 polypeptides (Δ, δ and ε), syk family tyrosine kinases (Syk, ZAP 70, etc.), src family tyrosine kinases (Lck, Fyn, Lyn, etc.) and other molecules involved in T-cell transduction, such as CD2, CD5 and CD28. The intracellular signalling domain may be human CD3 zeta signalling domain, FcyRIII, FcsRI, cytoplasmic tails of Fc receptors, immunoreceptor tyrosine-based activation motif (ITAM) bearing cytoplasmic receptors or combinations thereof.

Preferably, the intracellular signalling domain comprises the intracellular signalling domain of human CD3 zeta signalling domain. Preferably, the CD3 zeta signalling domain is in the same polypeptide chain as the antigen recognition domain which binds or specifically binds to ASGR. Suitably, the intracellular signalling domain may comprise the amino acid sequence shown as SEQ ID NO: 103, or a variant which is at least 80% identical to SEQ ID NO: 103.

Illustrative CD3 zeta signalling domain (SEQ ID
NO: 103):
RVKFSRSADAPAYQQGQNQLYNELNLGRREEYDVLDKRRGRDPEMGGKP
RRKNPQEGLYNELQKDKMAEAYSEIGMKGERRRGKGHDGLYQGLSTATK
DTYDALHMQALPPR

Suitably, the variant may be at least 85, 90, 95, 97, 98 or 99% identical to SEQ ID NO: 103.

The intracellular signalling domain of the CAR may comprise the CD28 signalling domain. Suitably, the intracellular signalling domain may comprise the amino acid sequence shown as SEQ ID NO: 104, or a variant which is at least 80% identical to SEQ ID NO: 104.

Illustrative CD28 signalling domain (SEQ ID
NO: 104):
RSKRSRLLHSDYMNMTPRRPGPTRKHYQPYAPPRDFAAYRS

Suitably, the variant may be at least 85, 90, 95, 97, 98 or 99% identical to SEQ ID NO: 104.

The intracellular signalling domain of the CAR may comprise the CD27 signalling domain. Suitably, the intracellular signalling domain may comprise the amino acid sequence shown as SEQ ID NO: 105, or a variant which is at least 80% identical to SEQ ID NO: 105.

Illustrative CD27 signalling domain (SEQ ID
NO: 105):
QRRKYRSNKGESPVEPAEPCHYSCPREEEGSTIPIQEDYRKPEPACSP

In one embodiment, the intracellular signalling domain comprises a signalling motif which has at least 85, 90, 95, 97, 98 or 99% identity to SEQ ID NO: 105.

Additional intracellular signalling domains will be apparent to those of skill in the art and may be used in connection with alternate embodiments of the invention.

The CAR may also comprise one or more co-stimulatory domains.

A “co-stimulatory domains” as used herein refers to an intracellular part of the CAR that may promote Treg function (e.g. immunosuppressive function), expansion, and/or persistence.

Accordingly, the CAR may comprise a compound endodomain comprising a fusion of the one or more co-stimulatory domains to that of an intracellular signalling domain e.g. CD3ζ. Such a compound endodomain may be referred to as a second generation CAR which can transmit an activating and co-stimulatory signal simultaneously after antigen recognition. The co-stimulatory domain most commonly used is that of CD28. This supplies the most potent co-stimulatory signal—namely immunological signal 2, which triggers Treg proliferation. Suitable co-stimulatory domains will be apparent to those of skill in the art.

Suitably, the one or more co-stimulatory domains may comprise the amino acid sequence shown as SEQ ID NO: 104 or 105, or a variant which is at least 80% (e.g. at least 85, 90, 95, 97, 98 or 99%) identical to SEQ ID NO: 104 or 105.

Suitably, the one or more co-stimulatory domains may comprise one or more TNF receptor family signalling domain, such as the signalling domain of OX40, 4-1BB, ICOS or TNFRSF25.

Illustrative sequences for OX40, 4-1BB, ICOS and TNFRSF25 signalling domains are shown below. The one or more co-stimulatory domains may comprise one or more of SEQ ID NOs: 106-109 or a variant which is at least 80% identical to one or more of SEQ ID NOs: 106-109.

Illustrative OX40 signalling domain (SEQ ID
NO: 106):
ALYLLRRDQRLPPDAHKPPGGGSFRTPIQEEQADAHSTLAKI
Illustrative 41BB signalling domain (SEQ ID
NO: 107):
KRGRKKLLYIFKQPFMRPVQTTQEEDGCSCRFPEEEEGGCEL
Illustrative ICOS signalling domain (SEQ ID
NO: 108):
CWLTKKKYSSSVHDPNGEYMFMRAVNTAKKSRLTDVTL
Illustrative TNFRSF25 signalling domain (SEQ ID
NO: 109):
TYTYRHCWPHKPLVTADEAGMEALTPPPATHLSPLDSAHTLLAPPDSSE
KICTVQLVGNSWTPGYPETQEALCPQVTWSWDQLPSRALGPAAAPTLSP
ESPAGSPAMMLQPGPQLYDVMDAVPARRWKEFVRTLGLREAEIEAVEVE
IGRFRDQQYEMLKRWRQQQPAGLGAVYAALERMGLDGCVEDLRSRLQRG
P

The one or more co-stimulatory domains may comprise a variant of one or more of OX40, 4-1BB, ICOS and TNFRSF25 signalling domains which has at least 85, 90, 95, 97, 98 or 99% identity to any one of SEQ ID NOs: 106-109.

The endodomain of the CAR may comprise the CD28 signalling domain and the CD3 zeta signalling domain. Suitably, the endodomain may comprise the amino acid sequence shown as SEQ ID NO: 110, or a variant which is at least 80% identical to SEQ ID NO: 110.

Illustrative CD28 signalling domain and CD3 zeta
signalling domain (SEQ ID NO: 110):
RSKRSRLLHSDYMNMTPRRPGPTRKHYQPYAPPRDFAAYRSRVKFSRSA
DAPAYQQGQNQLYNELNLGRREEYDVLDKRRGRDPEMGGKPRRKNPQEG
LYNELQKDKMAEAYSEIGMKGERRRGKGHDGLYQGLSTATKDTYDALHM
QALPPR

Suitably, the variant may be at least 85, 90, 95, 97, 98 or 99% identical to SEQ ID NO: 110.

In some embodiments the CAR comprises an endodomain which comprises a STAT5 association motif and a JAK1- and/or a JAK2-binding motif.

“Signal Transducer and Activator of Transcription 5” (STAT5) is a transcription factor involved in the IL-2 signalling pathway that plays a key role in Treg function, stability and survival by promoting the expression of genes such as FOXP3, IL2RA and BCLXL. In order to be functional and translocate into the nucleus, STAT5 needs to be phosphorylated. IL-2 ligation results in STAT5 phosphorylation by activating the Jak1 and Jak3 kinases via specific signalling domains present in the IL-2Rβ and IL-2Rγ chain, respectively. Although Jak1 (or Jak2) can phosphorylate STAT5 without the need of Jak3, STAT5 activity is increased by the transphosphorylation of both Jak1 and Jak3, which stabilizes their activity.

“STAT5 association motif” as used herein refers to an amino acid motif which comprises a tyrosine and is capable of binding a STAT5 polypeptide. Any method known in the art for determining protein:protein interactions may be used to determine whether an association motif is capable of binding to STAT5. For example, co-immunoprecipitation followed by western blot.

The present invention thus provides an engineered Treg comprising a CAR with the ability to bind to a liver-specific antigen, preferably asialoglycoprotein receptor (ASGR), which CAR also provides a STAT5-mediated pro-survival signal to the Treg exclusively upon CAR binding to the antigen. In particular, after antigen recognition, the CARs cluster and a signal is transmitted to the engineered Treg via the intracellular signalling domain (endodomain) of the CAR. When the CAR comprises an endodomain which comprises a STAT5 association motif and a JAK1- and/or a JAK2-binding motif, clustering of the CAR leads to STAT5 and JAK1 and/or JAK2 recruitment and activation; and thus provides a signal that enhances the function and the survival of the engineered Treg in an antigen-specific manner without being dependent on the availability of IL-2 in the microenvironment.

The engineered Tregs of the present invention with STAT5 signalling may be particularly effective in providing a survival advantage to the engineered CAR-Tregs after antigen recognition compared to the general T cell population of the subject. In particular, in the context of e.g. transplantation where the use of immunosuppressive drugs reduces the availability of IL-2, the STAT5 signalling of the CAR-Tregs provides additional survival and functional effects on the cells of the invention in an otherwise disadvantageous microenvironment.

Suitably, the CAR endodomain may comprise two or more STAT5 association motifs as defined herein. For example, the CAR endodomain may comprise two, three, four, five or more STAT5 association motifs as defined herein. Preferably, the CAR endodomain may comprise two or three STAT5 association motifs as defined herein.

Suitably, the STAT5 association motif may exist endogenously in a cytoplasmic domain of a transmembrane protein. For example, the STAT5 association motif may be from an interleukin receptor (IL) receptor endodomain or a hormone receptor.

The CAR endodomain may comprise an amino acid sequence selected from any chain of the interleukin receptors where STAT5 is a downstream component, for example, the cytoplasmic domain comprising amino acid numbers 266 to 551 of IL-2 receptor β chain (NCBI REFSEQ: NP_000869.1, SEQ ID NO: 111), amino acid numbers 265 to 459 of IL-7R a chain (NCBI REFSEQ: NP_002176.2, SEQ ID NO: 112), IL-7RA 2Y chain truncated (SEQ ID NO: 113), amino acid numbers 292 to 521 of IL-9R chain (NCBI REFSEQ: NP_002177.2, SEQ ID NO: 114), amino acid numbers 257 to 825 of IL-4R α chain (NCBI REFSEQ: NPJD00409.1, SEQ ID NO: 115), amino acid numbers 461 to 897 of IL-3R β chain (NCBI REFSEQ: NP_000386.1, SEQ ID NO: 116) or amino acid numbers 314 to 502 of IL-17R β chain (NCBI REFSEQ: NP_061195.2, SEQ ID NO: 117) may be used. The entire region of the cytoplasmic domain of interleukin receptor chain may be used.

-IL7RB (AA 265 to 551 of NP_000869.1)
SEQ ID NO: 111
NCRNTGPWLKKVLKCNTPDPSKFFSQLSSEHGGDVQKWLSSPFPSSSFSPGGLAPEISPL
EVLERDKVTQLLLQQDKVPEPASLSSNHSLTSCFTNQGYFFFHLPDALEIEACQVYFTYDPY
SEEDPDEGVAGAPTGSSPQPLQPLSGEDDAYCTFPSRDDLLLFSPSLLGGPSPPSTAPGG
SGAGEERMPPSLQERVPRDWDPQPLGPPTPGVPDLVDFQPPPELVLREAGEEVPDAGPR
EGVSFPWSRPPGQGEFRALNARLPLNTDAYLSLQELQGQDPTHLV
-IL7RA (AA 265 to 459 of NP_002176.2)
SEQ ID NO: 112
KKRIKPIVWPSLPDHKKTLEHLCKKPRKNLNVSFNPESFLDCQIHRVDDIQARDEVEGFLQD
TFPQQLEESEKQRLGGDVQSPNCPSEDVVITPESFGRDSSLTCLAGNVSACDAPILSSSRS
LDCRESGKNGPHVYQDLLLSLGTTNSTLPPPFSLQSGILTLNPVAQGQPILTSLGSNQEEAY
VTMSSFYQNQ
-IL7RA 2Y truncated:
SEQ ID NO: 113
KKRIKPIVWPSLPDHKKTLEHLCKKPRKNLNVSFNPESFLDCQIHRVDDIQARDEVEGFLQD
TFPQQPILTSLGSNQEEAYVTMSSFYQNQ
-IL9R (AA 292 to 521of NP_002177.2)
SEQ ID NO: 114
KLSPRVKRIFYQNVPSPAMFFQPLYSVHNGNFQTWMGAHGAGVLLSQDCAGTPQGALEP
CVQEATALLTCGPARPWKSVALEEEQEGPGTRLPGNLSSEDVLPAGCTEWRVQTLAYLPQ
EDWAPTSLTRPAPPDSEGSRSSSSSSSSNNNNYCALGCYGGWHLSALPGNTQSSGPIPAL
ACGLSCDHQGLETQQGVAWLAGHCQRPGLHEDLQGMLLPSVLSKARSWTF
-IL4RA (AA 257 to 825 of NPJD00409.1)
SEQ ID NO: 115
KIKKEWWDQIPNPARSRLVAIIIQDAQGSQWEKRSRGQEPAKCPHWKNCLTKLLPCFLEHN
MKRDEDPHKAAKEMPFQGSGKSAWCPVEISKTVLWPESISVVRCVELFEAPVECEEEEEV
EEEKGSFCASPESSRDDFQEGREGIVARLTESLFLDLLGEENGGFCQQDMGESCLLPPSG
STSAHMPWDEFPSAGPKEAPPWGKEQPLHLEPSPPASPTQSPDNLTCTETPLVIAGNPAY
RSFSNSLSQSPCPRELGPDPLLARHLEEVEPEMPCVPQLSEPTTVPQPEPETWEQILRRNV
LQHGAAAAPVSAPTSGYQEFVHAVEQGGTQASAVVGLGPPGEAGYKAFSSLLASSAVSPE
KCGFGASSGEEGYKPFQDLIPGCPGDPAPVPVPLFTFGLDREPPRSPQSSHLPSSSPEHL
GLEPGEKVEDMPKPPLPQEQATDPLVDSLGSGIVYSALTCHLCGHLKQCHGQEDGGQTPV
MASPCCGCCCGDRSSPPTTPLRAPDPSPGGVPLEASLCPASLAPSGISEKSKSSSSFHPAP
GNAQSSSQTPKIVNFVSVGPTYMRVS
-IL3RB (AA 461 to 897 of NP_000386.1)
SEQ ID NO: 116
RFCGIYGYRLRRKWEEKIPNPSKSHLFQNGSAELWPPGSMSAFTSGSPPHQGPWGSRFP
ELEGVFPVGFGDSEVSPLTIEDPKHVCDPPSGPDTTPAASDLPTEQPPSPQPGPPAASHTP
EKQASSFDFNGPYLGPPHSRSLPDILGQPEPPQEGGSQKSPPPGSLEYLCLPAGGQVQLV
PLAQAMGPGQAVEVERRPSQGAAGSPSLESGGGPAPPALGPRVGGQDQKDSPVAIPMSS
GDTEDPGVASGYVSSADLVFTPNSGASSVSLVPSLGLPSDQTPSLCPGLASGPPGAPGPV
KSGFEGYVELPPIEGRSPRSPRNNPVPPEAKSPVLNPGERPADVSPTSPQPEGLLVLQQV
GDYCFLPGLGPGPLSLRSKPSSPGPGPEIKNLDQAFQVKKPPGQAVPQVPVIQLFKALKQQ
DYLSLPPWEVNKPGEVC
-IL17RB (AA 314 to 502 of NP_061195.2)
SEQ ID NO: 117
RHERIKKTSFSTTTLLPPIKVLVVYPSEICFHHTICYFTEFLQNHCRSEVILEKWQKKKIAEMG
PVQWLATQKKAADKVVFLLSNDVNSVCDGTCGKSEGSPSENSQDLFPLAFNLFCSDLRSQI
HLHKYVVVYFREIDTKDDYNALSVCPKYHLMKDATAFCAELLHVKQQVSAGKRSQACHDG
CCSL

The CAR endodomain may comprise a STAT5 association motif that comprises an amino acid sequence shown as one of SEQ ID NOs: 111-117, or a variant which is at least 80, 85, 90, 95, 96, 97, 98 or 99% identical to one of SEQ ID NOs: 111-117. For example, the variant may be capable of binding STAT5 to at least 10%, at least 20%, at least 30%, at least 40%, at least 50%, at least 60%, at least 70%, at least 80%, or at least 90% of the level of an amino acid sequence shown as one of SEQ ID NO: 111-117. The variant may be capable of binding STAT5 to a similar or the same level as one of SEQ ID NO: 111-117 or may be capable of binding STAT5 to a greater level than an amino acid sequence shown as one of SEQ ID NO: 111-117 (e.g. increased by at least 10%, at least 20%, at least 30%, at least 40% or at least 50%).

For example, the STAT5 association motif may be from IL2Rβ, IL7Rα, IL-3Rβ (CSF2RB), IL-9R, IL-4Rα, IL-17Rβ, erythropoietin receptor, thrombopoietin receptor, growth hormone receptor and prolactin receptor.

The STAT5 association motif may comprise the amino acid motif YXXF/L (SEQ ID NO: 118); wherein X is any amino acid.

Suitably, the STAT5 association motif may comprise the amino acid motif YCTF (SEQ ID NO: 119), YFFF (SEQ ID NO: 120), YLSL (SEQ ID NO: 121), or YLSLQ (SEQ ID NO: 122).

Suitably, the STAT5 association motif may comprise the amino acid motif YLSLQ (SEQ ID NO: 122).

The CAR endodomain may comprise one or more STAT5 association motif comprising the amino acid motif YCTF (SEQ ID NO: 119), YFFF (SEQ ID NO: 120), YLSL (SEQ ID NO: 121), and/or YLSLQ (SEQ ID NO: 122).

The CAR endodomain may comprise a first STAT5 association motif comprising the amino acid motif YLSLQ (SEQ ID NO: 122) and a second STAT5 association motif comprising the amino acid motif YCTF (SEQ ID NO: 119) or YFFF (SEQ ID NO: 120).

The CAR endodomain may comprise the following STAT5 association motifs: YLSLQ (SEQ ID NO: 122), YCTF (SEQ ID NO: 119) and YFFF (SEQ ID NO: 120).

“JAK1- and/or a JAK2-binding motif” as used herein refers to BOX motif which allows for tyrosine kinase JAK1 and/or JAK2 association. Suitable JAK1- and JAK2-binding motifs are described, for example, by Ferrao & Lupardus (Frontiers in Endocrinology; 2017; 8(71); which is incorporated herein by reference).

Any method known in the art for determining protein:protein interactions may be used to determine whether a motif is capable of binding to JAK1 and/or JAK2. For example, co-immunoprecipitation followed by western blot.

The JAK1 and/or JAK2-binding motif may occur endogenously in a cytoplasmic domain of a transmembrane protein.

For example, the JAK1 and/or JAK2-binding motif may be from IFNLR1, IFNAR, IFNGR1, IL10RA, IL20RA, IL22RA, IFNGR2 or IL10RB.

The JAK1-binding motif may comprise an amino acid motif shown as SEQ ID NOs: 123-129 or a variant thereof which is capable of binding JAK1. For example, the variant may be capable of binding JAK1 to at least 10%, at least 20%, at least 30%, at least 40%, at least 50%, at least 60%, at least 70%, at least 80%, or at least 90% of the level of an amino acid sequence shown as one of SEQ ID NO: 123-129. The variant may be capable of binding JAK1 to a similar or the same level as one of SEQ ID NO: 123-129 or may be capable of binding JAK1 to a greater level than an amino acid sequence shown as one of SEQ ID NO: 123-129 (e.g. increased by at least 10%, at least 20%, at least 30%, at least 40% or at least 50%).

| | |
|---|---|
| JAK1-binding motif 1 | KVLKCNTPDPSKFFSQLSSEHGGDVQKWLSSPFPSSSFSPGGLAPE |
| (SEQ ID NO: 123) | ISPLEVLERDK |
| JAK1-binding motif 2 | NPWFQRAKMPRALDFSGHTHPVATFQPSRPESVNDLFLCPQKELT |
| (SEQ ID NO: 124) | |
| JAK1-binding motif 3 | GYICLRNSLPKVLNFHNFLAWPFPNLPPLEAMDMVEVIYINR |
| (SEQ ID NO: 125) | |
| JAK1-binding motif 4 | PLKEKSIILPKSLISVVRSATLETKPESKYVSLITSYQPFSL |
| (SEQ ID NO: 126) | |
| JAK1-binding motif 5 | RRRKKLPSVLLFKKPSPFIFISQRPSPETQDTIHPLDEEAFLK |
| (SEQ ID NO: 127) | |
| JAK1-binding motif 6 | YIHVGKEKHPANLILIYGNEFDKRFFVPAEKIVINFITLNISDDS |
| (SEQ ID NO: 128) | |
| JAK1-binding motif 7 | RYVTKPPAPPNSLNVQRVLTFQPLRFIQEHVLIPVFDLSGP |
| (SEQ ID NO: 129) | |

The variant may comprise one, two or three amino acid differences compared to any of SEQ ID NOs: 123-129 and retain the ability to bind JAK1.

The variant may be at least 80, 85, 90, 95, 96, 97, 98 or 99% identical to any one of SEQ ID NOs: 123-129.

In a preferred embodiment, the JAK1-binding domain comprises SEQ ID NO: 123 or a variant thereof which is capable of binding JAK1.

The JAK2-binding motif may comprise an amino acid motif shown as SEQ ID NOs: 130-132 or a variant therefore which is capable of binding JAK2. For example, the variant may be capable of binding JAK2 to at least 10%, at least 20%, at least 30%, at least 40%, at least 50%, at least 60%, at least 70%, at least 80%, or at least 90% of the level of an amino acid sequence shown as one of SEQ ID NO: 130-132. The variant may be capable of binding JAK2 to a similar or the same level as one of SEQ ID NO: 130-132 or may be capable of binding JAK2 to a greater level than an amino acid sequence shown as one of SEQ ID NO: 130-132 (e.g. increased by at least 10%, at least 20%, at least 30%, at least 40% or at least 50%).

| | |
|---|---|
| JAK2-binding motif 1 | NYVFFPSLKPSSSIDEYFSEQPLKNLLLSTSEEQIEKCFIIEN |
| (SEQ ID NO: 130) | |
| JAK2-binding motif 2 | YWFHTPPSIPLQIEEYLKDPTQPILEALDKDSSPKDDVWDSVSIISFPE |
| (SEQ ID NO: 131) | |
| JAK2-binding motif 3 | YAFSPRNSLPQHLKEFLGHPHHNTLLFFSFPLSDENDVFDKLSVIAED |
| (SEQ ID NO: 132) | SES |

The variant may comprise one, two or three amino acid differences compared to any of SEQ ID NOs: 130-132 and retain the ability to bind JAK2.

The variant may be at least 80, 85, 90, 95, 96, 97, 98 or 99% identical to any one of SEQ ID NOs: 130-132.

Any method known in the art for determining protein:protein interactions may be used to determine whether a JAK1- or JAK2-binding motif is capable of binding to a JAK1 or JAK2. For example, co-immunoprecipitation followed by western blot.

Suitably, the endodomain of the CAR described herein may not comprise a “Signal Transducer and Activator of Transcription 3” (STAT3) association motif.

STAT3 has been described as a detrimental signal for the stability and function of Tregs. For example, STAT3 signalling promotes the expression of pro-inflammatory genes such IL17, IL21, and IL22. As such, the use of a CAR which does not comprise a STAT3 association motif provides particular advantages in the context of an engineered Treg of the present invention.

A STAT3 association motif may comprise the amino acid sequence YXXQ (SEQ ID NO: 133), wherein “X” is any amino acid, and be capable of binding STAT3. Any method known in the art for determining protein:protein interactions may be used to determine whether a STAT3 association motif is capable of binding to STAT3. For example, co-immunoprecipitation followed by western blot.

Suitably, the CAR endodomain does not comprise the amino acid sequence YXXQ (SEQ ID NO: 133), wherein “X” is any amino acid.

“STAT3 association motif” may refer to an amino acid motif which comprises a tyrosine and is capable of functionally binding (i.e. leading to activation of) a STAT3 polypeptide, when present in a Treg.

Suitably, the CAR endodomain does not comprise an amino acid motif which comprises a tyrosine and is capable of binding a STAT3 polypeptide. For example, suitably the CAR endodomain does not comprise an amino acid motif which comprises a tyrosine and is capable of functionally binding (i.e. leading to activation of) a STAT3 polypeptide, when present in a Treg.

Suitably, the endodomain of the present CAR is not capable of inducing productive STAT3 and/or STAT1 signalling when expressed in a Treg. In other words, when expressed in a Treg, the present CAR is not capable of functionally binding and/or inducing phosphorylation and activation of STAT3 and/or STAT1. Suitably, the CAR is not capable of inducing STAT3 and/or STAT1 dependent transcriptional activation when expressed in a Treg.

Suitably, the IL2Rβ endodomain portion of the CAR endodomain does not comprise a STAT3 association motif as defined herein.

Suitably, the CAR endodomain may comprise an IL2Rβ endodomain. Suitably, the CAR endodomain may comprise the amino acid sequence shown as SEQ ID NO: 134; or a variant which has at least 80% sequence identity to SEQ ID NO: 134.

Illustrative IL2Rβ endodomain
(SEQ ID NO: 134)
NCRNTGPWLKKVLKCNTPDPSKFFSQLSSEHGGDVQKWLSSPFPSSSFS
PGGLAPEISPLEVLERDKVTQLLLQQDKVPEPASLSSNHSLTSCFTNQG
YFFFHLPDALEIEACQVYFTYDPYSEEDPDEGVAGAPTGSSPQPLQPLS
GEDDAYCTFPSRDDLLLFSPSLLGGPSPPSTAPGGSGAGEERMPPSLQE
RVPRDWDPQPLGPPTPGVPDLVDFQPPPELVLREAGEEVPDAGPREGVS
FPWSRPPGQGEFRALNARLPLNTDAYLSLQELQGQDPTHLV

The variant may be at least 80, 85, 90, 95, 96, 97, 98 or 99% identical to SEQ ID NO: 134.

Suitably, the CAR endodomain may comprise a truncated IL2Rβ endodomain. Suitably, the CAR endodomain may comprise the amino acid sequence shown as SEQ ID NO: 135 or SEQ ID NO: 136; or a variant of SEQ ID NO: 135 or SEQ ID NO: 136 which has at least 80% sequence identity thereto.

Illustrative IL2RB truncated-Y510
(SEQ ID NO: 135)
NCRNTGPWLKKVLKCNTPDPSKFFSQLSSEHGGDVQKWLSSPFPSSSFS
PGGLAPEISPLEVLERDKVTQLLPLNTDAYLSLQELQGQDPTHLV
Illustrative IL2RB truncated-Y510 & Y392
(SEQ ID NO: 136)
NCRNTGPWLKKVLKCNTPDPSKFFSQLSSEHGGDVQKWLSSPFPSSSFS
PGGLAPEISPLEVLERDKVTQLLDAYCTFPSRDDLLLFSPSLLGGPSPP
STAPGGSGAGEERMPPSLQERVPRDWDPQPLGPPTPGVPDLVDFQPPPE
LVLREAGEEVPDAGPREGVSFPWSRPPGQGEFRALNARLPLNTDAYLSL
QELQGQDPTHLV

The variant may be at least 80, 85, 90, 95, 96, 97, 98 or 99% identical to SEQ ID NO: 135 or SEQ ID NO: 136.

STAT5 activity is increased by the transphosphorylation of both a Jak1/2 and Jak3, as this stabilizes their activity. Suitably, the CAR endodomain as described herein may further comprise a JAK3-binding motif. “JAK3-binding motif” as used herein refers to BOX motif which allows for tyrosine kinase JAK3. Suitable JAK3-binding motifs are described, for example, by Ferrao & Lupardus (Frontiers in Endocrinology; 2017; 8(71); which is incorporated herein by reference).

Any method known in the art for determining protein:protein interactions may be used to determine whether a motif is capable of binding to JAK3. For example, co-immunoprecipitation followed by western blot.

The JAK3-binding motif may occur endogenously in a cytoplasmic domain of a transmembrane protein.

For example, the JAK3-binding motif may be from an IL-2Rγ polypeptide.

The JAK3-binding motif may comprise an amino acid motif shown as JSEQ ID NO: 137 or 138, or a variant therefore which is capable of binding JAK3.

JAK3-binding motif 1
(SEQ ID NO: 137)
ERTMPRIPTLKNLEDLVTEYHGNFSAWSGVSKGLAESLQPDYSERLCLV
SEI
JAK3-binding motif 2
(SEQ ID NO: 138)
ERTMPRIPTLKNLEDLVTEYHGNFSAWSGVSKGLAESLQPDYSERLCLV
SEIPPKGGALGEGPGASPCNQHSPYWAPPCYTLKPET

The variant may be at least 80, 85, 90, 95, 96, 97, 98 or 99% identical to SEQ ID NO: 137 or 138.

In some embodiments the CAR comprises an endodomain which comprises a STAT5 association motif, a JAK1- and/or a JAK2-binding motif, and a JAK3-binding motif.

In a preferred embodiment, the CAR endodomain comprises one or more JAK1-binding domains and at least one JAK3-binding domain.

Suitably, the CAR endodomain may comprise SEQ ID NO: 139 or a variant which has at least 85, 90, 95, 97, 98 or 99% identity to SEQ ID NO: 139.

(illustrative endodomain sequence comprising CD28,
IL2RG-T52, IL2RB-Y510, and CD3 zeta signalling
domains)
SEQ ID NO: 139
RSKRSRLLHSDYMNMTPRRPGPTRKHYQPYAPPRDFAAYRSERTMPRIP
TLKNLEDLVTEYHGNFSAWSGVSKGLAESLQPDYSERLCLVSEINCRNT
GPWLKKVLKCNTPDPSKFFSQLSSEHGGDVQKWLSSPFPSSSFSPGGLA
PEISPLEVLERDKVTQLLPLNTDAYLSLQELQGQDPTHLVRVKFSRSAD
APAYQQGQNQLYNELNLGRREEYDVLDKRRGRDPEMGGKPRRKNPQEGL
YNELQKDKMAEAYSEIGMKGERRRGKGHDGLYQGLSTATKDTYDALHMQ
ALPPR

Suitably, the CAR endodomain may comprise SEQ ID NO: 140 or a variant which has at least 85, 90, 95, 97, 98 or 99% identity to SEQ ID NO: 140.

(illustrative endodomain sequence comprising CD28,
IL2RG-T52, IL7RA-2Y, and CD3 zeta signalling
domains)
SEQ ID NO: 140
RSKRSRLLHSDYMNMTPRRPGPTRKHYQPYAPPRDFAAYRSERTMPRIP
TLKNLEDLVTEYHGNFSAWSGVSKGLAESLQPDYSERLCLVSEIKKRIK
PIVWPSLPDHKKTLEHLCKKPRKNLNVSFNPESFLDCQIHRVDDIQARD
EVEGFLQDTFPQQPILTSLGSNQEEAYVTMSSFYQNQRVKFSRSADAPA
YQQGQNQLYNELNLGRREEYDVLDKRRGRDPEMGGKPRRKNPQEGLYNE
LQKDKMAEAYSEIGMKGERRRGKGHDGLYQGLSTATKDTYDALHMQALP
PRGSGATNFSLLKQAGDVEENPG

Other Domains

In some embodiments the CAR comprises one or more signal peptides.

The CAR may comprise a leader sequence which targets it to the endoplasmic reticulum pathway for expression on the cell surface. An illustrative leader sequence is CD8 leader (SEQ ID NO: 141).

CD8 leader (SEQ ID NO: 141):
MALPVTALLLPLALLLHAARP

In some embodiments the CAR comprise one or more reporter domains, optionally in combination with a self-cleaving or cleavage domain.

Suitable reporter domains are well known in the art and include, but are not limited to, fluorescent proteins—such as GFP. The use of a reporter domain is advantageous as it allows a Treg in which a polynucleotide or vector of the present invention has been successfully introduced (such that the encoded CAR is expressed) to be selected and isolated from a starting cell population using common methods, e.g. flow cytometry.

Suitably, the reporter domain may be a fluorescent protein, for example GFP, YFP, RFP, tdTomato, dsRed, or variants thereof. In some embodiments the fluorescent protein is GFP or a GFP variant. Suitably, the GFP variant may comprise the amino acid sequence shown as SEQ ID NO: 142, or a variant which is at least 80% identical to SEQ ID NO: 142.

Illustrative eGFP (SEQ ID NO: 142):
MVSKGEELFTGVVPILVELDGDVNGHKFSVSGEGEGDATYGKLTLKFIC
TTGKLPVPWPTLVTTLTYGVQCFSRYPDHMKQHDFFKSAMPEGYVQERT
IFFKDDGNYKTRAEVKFEGDTLVNRIELKGIDFKEDGNILGHKLEYNYN
SHNVYIMADKQKNGIKVNFKIRHNIEDGSVQLADHYQQNTPIGDGPVLL
PDNHYLSTQSKLSKDPNEKRDHMVLLEFVTAAGITLGMDELYK

Suitably, the variant may be at least 85, 90, 95, 97, 98 or 99% identical to SEQ ID NO: 142.

Suitably, the reporter domain may be a luciferase-based reporter, a PET reporter (e.g. Sodium Iodide Symporter (NIS)), or a membrane protein (e.g. CD34, low-affinity nerve growth factor receptor (LNGFR)).

The nucleic acid sequences encoding the CAR and the reporter domain may be separated by a co-expression site which enables expression of each polypeptide as a discrete entity. Suitable co-expression sites are known in the art and include, for example, internal ribosome entry sites (IRES) and self-cleaving peptides.

Accordingly, the CAR may further comprise a self-cleaving or cleavage domain. Such sequences may either auto-cleave during protein production or may be cleaved by common enzymes present in the cell. Accordingly, inclusion of such self-cleaving or cleavage domains in the polypeptide sequence enables a first and a second polypeptide to be expressed as a single polypeptide, which is subsequently cleaved to provide discrete, separated functional polypeptides. Suitable self-cleaving or cleavage domains include, but are not limited to P2A peptide, T2A peptide, E2A peptide, F2A peptide, and furin site (SEQ ID NOs: 143-148).

P2A peptide-cleavage domain:
(SEQ ID NO: 143)
GSGATNFSLLKQAGDVEENPGP
T2A peptide-cleavage domain:
(SEQ ID NO: 144)
GSGEGRGSLLTCGDVEENPGP
E2A peptide-cleavage domain:
(SEQ ID NO: 145)
GSGQCTNYALLKLAGDVESNPGP
F2A peptide-cleavage domain:
(SEQ ID NO: 146)
GSGVKQTLNFDLLKLAGDVESNPGP
Furin site-cleavage domain:
(SEQ ID NO: 147)
RXXR (preferentially: RRKR (SEQ ID NO: 148))

Illustrative CARs

Illustrative CARs for use in the present invention are shown below. The CAR may comprise a sequence which has at least 70, 80, 85, 90, 95, 97, 98, 99% or 100% identity to one or more of SEQ ID NOs: 149-152 or 167-169. Preferably any such variant has at least partial functionality as compared to SEQ ID NOs: 149-152 or 167-169. For example, the variant may have at least 10%, at least 20%, at least 30%, at least 40%, at least 50%, at least 60%, at least 70%, at least 80%, or at least 90% of the function of an amino acid sequence shown as one of SEQ ID NO: 149-152 or 167-169. The variant may have functionality similar to or the same level as one of SEQ ID NO: 149-152 or 167-169 or may have functionality of a greater level than an amino acid sequence shown as one of SEQ ID NO: 149-152 or 167-169 (e.g. increased by at least 10%, at least 20%, at least 30%, at least 40% or at least 50%).

-Illustrative CAR 1: CAR containing CD8 leader, ASGR1 VH, CD8α hinge,
CD28 transmembrane, CD28 cytoplasmic, CD3z cytoplasmic
SEQ ID NO: 149
MALPVTALLLPLALLLHAARPEVQLLESGGGLVQPGGSLRLSCAASGFTFEKYAMAWRQA
PGKGLEWSRISARGVTTYYADSVKGRFTISRDNSKNTLYLQMNSLRAEDTAVYYCAKHKR
HEHTRFDSWGQGTLVTVSSTTTPAPRPPTPAPTIASQPLSLRPEACRPAAGGAVHTRGLDF
ACDFWVLVVVGGVLACYSLLVTVAFIIFWVRSKRSRLLHSDYMNMTPRRPGPTRKHYQPYA
PPRDFAAYRSRVKFSRSADAPAYQQGQNQLYNELNLGRREEYDVLDKRRGRDPEMGGKP
RRKNPQEGLYNELQKDKMAEAYSEIGMKGERRRGKGHDGLYQGLSTATKDTYDALHMQA
LPPR
-Illustrative CAR 2: CAR containing CD8 leader, ASGR1 VH, CD28 hinge
with c-Myc tag, CD28 transmembrane, CD28 cytoplasmic, CD3z cytoplasmic
SEQ ID NO: 150
MALPVTALLLPLALLLHAARPEVQLLESGGGLVQPGGSLRLSCAASGFTFEKYAMAWRQA
PGKGLEWVSRISARGVTTYYADSVKGRFTISRDNSKNTLYLQMNSLRAEDTAVYYCAKHKR
HEHTRFDSWGQGTLVTVSSAAAIEVEQKLISEEDLLDNEKSNGTIIHVKGKHLCPSPLFPGP
SKPFWVLVWGGVLACYSLLVTVAFIIFWVRSKRSRLLHSDYMNMTPRRPGPTRKHYQPYA
PPRDFAAYRSRVKFSRSADAPAYQQGQNQLYNELNLGRREEYDVLDKRRGRDPEMGGKP
RRKNPQEGLYNELQKDKMAEAYSEIGMKGERRRGKGHDGLYQGLSTATKDTYDALHMQA
LPPR
-Illustrative CAR 3: CAR containing CD8 leader, ASGR1 VH, CD8α hinge,
CD28 transmembrane, CD28 cytoplasmic, CD3z cytoplasmic, FP2A domain, GFP
SEQ ID NO: 151
MALPVTALLLPLALLLHAARPEVQLLESGGGLVQPGGSLRLSCAASGFTFEKYAMAWVRQA
PGKGLEWVSRISARGVTTYYADSVKGRFTISRDNSKNTLYLQMNSLRAEDTAVYYCAKHKR
HEHTRFDSWGQGTLVTVSSTTTPAPRPPTPAPTIASQPLSLRPEACRPAAGGAVHTRGLDF
ACDFWVLVWGGVLACYSLLVTVAFIIFWVRSKRSRLLHSDYMNMTPRRPGPTRKHYQPYA
PPRDFAAYRSRVKFSRSADAPAYQQGQNQLYNELNLGRREEYDVLDKRRGRDPEMGGKP
RRKNPQEGLYNELQKDKMAEAYSEIGMKGERRRGKGHDGLYQGLSTATKDTYDALHMQA
LPPRRRKRGSGATNFSLLKQAGDVEENPGPTRGGGATMVSKGEELFTGVVPILVELDGDV
NGHKFSVSGEGEGDATYGKLTLKFICTTGKLPVPWPTLVTTLTYGVQCFSRYPDHMKQHD
FFKSAMPEGYVQERTIFFKDDGNYKTRAEVKFEGDTLVNRIELKGIDFKEDGNILGHKLEYN
YNSHNVYIMADKQKNGIKVNFKIRHNIEDGSVQLADHYQQNTPIGDGPVLLPDNHYLSTQSK
LSKDPNEKRDHMVLLEFVTAAGITLGMDELYK
-Illustrative CAR 4: CAR containing CD8 leader, ASGR1 VH, CD8α hinge,
CD28 transmembrane, CD28 cytoplasmic, CD3z cytoplasmic, FP2A domain, GFP
SEQ ID NO: 152
MALPVTALLLPLALLLHAARPEVQLLESGGGLVQPGGSLRLSCAASGFTFEKYAMAWVRQA
PGKGLEWVSRISARGVTTYYADSVKGRFTISRDNSKNTLYLQMNSLRAEDTAVYYCAKHKR
HEHTRFDSWGQGTLVTVSSAAAIEVEQKLISEEDLLDNEKSNGTIIHVKGKHLCPSPLFPGP
SKPFWVLVWGGVLACYSLLVTVAFIIFWVRSKRSRLLHSDYMNMTPRRPGPTRKHYQPYA
PPRDFAAYRSRVKFSRSADAPAYQQGQNQLYNELNLGRREEYDVLDKRRGRDPEMGGKP
RRKNPQEGLYNELQKDKMAEAYSEIGMKGERRRGKGHDGLYQGLSTATKDTYDALHMQA
LPPRRRKRGSGATNFSLLKQAGDVEENPGPTRGGGATMVSKGEELFTGVVPILVELDGDV
NGHKFSVSGEGEGDATYGKLTLKFICTTGKLPVPWPTLVTTLTYGVQCFSRYPDHMKQHD
FFKSAMPEGYVQERTIFFKDDGNYKTRAEVKFEGDTLVNRIELKGIDFKEDGNILGHKLEYN
YNSHNVYIMADKQKNGIKVNFKIRHNIEDGSVQLADHYQQNTPIGDGPVLLPDNHYLSTQSK
LSKDPNEKRDHMVLLEFVTAAGITLGMDELYK
-Illustrative CAR 5: CAR containing CD8 leader, ASGR1 VH (VH1, SEQ ID
NO: 74), linker, ASGR1 VL (VK1, SEQ ID NO: 78), CD28 hinge with c-Myc tag,
CD28 transmembrane, CD28 cytoplasmic, CD3z cytoplasmic
SEQ ID NO: 167
MALPVTALLLPLALLLHAARPEVQLLESGGGLVQPGGSLRLSCAASGFTFEKYAMAWVRQAPGKGLEW
VSRISARGVTTYYADSVKGRFTISRDNSKNTLYLQMNSLRAEDTAVYYCAKHKRHEHTRFDSWGQGTL
VTVSSLVTVSSGGGGSGGGGSGGGGSDIQMTQSPSSLSASVGDRVTITCRASQAIGRWLLWYQQKPGK
APKLLIGPGSRLRSGVPSRFSGSGSGTDFTLTISSLQPEDFVTYYCQQAYAWPPTFGQGTKVEIKRAA
AIEVEQKLISEEDLLDNEKSNGTIIHVKGKHLCPSPLFPGPSKPFWVLVVVGGVLACYSLLVTVAFII
FWVRSKRSRLLHSDYMNMTPRRPGPTRKHYQPYAPPRDFAAYRSRVKFSRSADAPAYQQGQNQLYNEL
NLGRREEYDVLDKRRGRDPEMGGKPRRKNPQEGLYNELQKDKMAEAYSEIGMKGERRRGKGHDGLYQG
LSTATKDTYDALHMQALPPR
-Illustrative CAR 6: CAR containing CD8 leader, ASGR1 VH (VH2, SEQ ID
NO: 75), linker, ASGR1 VL (VK2, SEQ ID NO: 79), CD28 hinge with c-Myc tag,
CD28 transmembrane, CD28 cytoplasmic, CD3z cytoplasmic
SEQ ID NO: 168
MALPVTALLLPLALLLHAARPELQLLEFGGGLVQPGGSLRLSCTTSGFTFSRYTMGWVRQAPGKGLEW
VSAIGPPGSNTYYADSVKGRFTISRDNSKNTLYLQMNSLRAEDTAVYYCAKWVMLRGRFDYWGQGTLV
TVSSLVTVSSGGGGSGGGGSGGGGSDIQMTQSPSSLSASVGDRVTITCRASQAIGRWLLWYQQKPGKA
PKHLIGPGSRLQSGVPSRFSGSGSGTDFTLTISSLQPEDFATYYCQQAYQLPVTFGQGTKVEIKRAAA
IEVEQKLISEEDLLDNEKSNGTIIHVKGKHLCPSPLFPGPSKPFWVLVVVGGVLACYSLLVTVAFIIF
WVRSKRSRLLHSDYMNMTPRRPGPTRKHYQPYAPPRDFAAYRSRVKFSRSADAPAYQQGQNQLYNELN
LGRREEYDVLDKRRGRDPEMGGKPRRKNPQEGLYNELQKDKMAEAYSEIGMKGERRRGKGHDGLYQGL
STATKDTYDALHMQALPPR
-Illustrative CAR 7: CAR containing CD8 leader, ASGR1 VH (VH3, SEQ ID
NO: 76), linker, ASGR1 VL (VK3, SEQ ID NO: 80), CD28 hinge with c-Myc tag,
CD28 transmembrane, CD28 cytoplasmic, CD3z cytoplasmic
SEQ ID NO: 169
MALPVTALLLPLALLLHAARPEVQLLESGGGLVQPGGSLRLSCAASGFTFEDYGMGWVRQAPGKGLEW
VSAIGRNGSQTYYADSVKGRFTISRDNSKNTLYLQMNSLRAEDTAVYYCAKLRRGRGLNTFTLDYWGQ
GTLVTVSSLVTVSSGGGGSGGGGSGGGGSDIQMTQSPSSLSASVGDRVTITCRASQAIGRWLLWYQQK
PGKAPKLLIGPGSRLQSGVPSRFSGSGSGTDFTLTIGSLQPEDFATYYCQQAYSLPPTFGQGTKVEIK
RAAAIEVEQKLISEEDLLDNEKSNGTIIHVKGKHLCPSPLFPGPSKPFWVLVVVGGVLACYSLLVTVA
FIIFWVRSKRSRLLHSDYMNMTPRRPGPTRKHYQPYAPPRDFAAYRSRVKFSRSADAPAYQQGQNQLY
NELNLGRREEYDVLDKRRGRDPEMGGKPRRKNPQEGLYNELQKDKMAEAYSEIGMKGERRRGKGHDGL
YOGLSTATKDTYDALHMQALPPR

Variants, Derivatives and Fragments

In addition to the specific proteins, peptides and nucleotides mentioned herein, the present invention also encompasses the use of derivatives, variants and fragments thereof.

The term “derivative” as used herein, in relation to proteins or polypeptides of the present invention includes any substitution of, variation of, modification of, replacement of, deletion of and/or addition of one (or more) amino acid residues from or to the sequence providing that the resultant protein or polypeptide retains the desired function (for example, where the derivative or variant is an antigen binding domain, the desired function may be the ability of the antigen binding domain to bind its target antigen, or where the derivative or variant is a signalling domain, the desired function may be the ability of that domain to signal (e.g. activate or inactivate a downstream molecule). The variant or derivative may have at least 10%, at least 20%, at least 30%, at least 40%, at least 50%, at least 60%, at least 70%, at least 80%, or at least 90% function as compared to the corresponding reference sequence; a similar or the same level of function as compared to the corresponding reference sequence; or an increased level of function as compared to the corresponding reference sequence, for example function increased by at least 10%, at least 20%, at least 30%, at least 40%, or at least 50% compared to an unmodified sequence.

Typically, amino acid substitutions may be made, for example from 1, 2 or 3 to 10 or 20 substitutions provided that the modified sequence retains the required activity or ability e.g.

at least 10%, at least 20%, at least 30%, at least 40%, at least 50%, at least 60%, at least 70%, at least 80%, or at least 90% activity as compared to the corresponding reference sequence; a similar or the same level of activity as compared to an the corresponding reference sequence; or an increased level of activity as compared to the corresponding reference sequence, for example activity increased by at least 10%, at least 20%, at least 30%, at least 40%, or at least 50% compared to an unmodified sequence. Amino acid substitutions may include the use of non-naturally occurring analogues.

Proteins or peptides used in the present invention may also have deletions, insertions or substitutions of amino acid residues which produce a silent change and result in a functionally equivalent protein. Deliberate amino acid substitutions may be made on the basis of similarity in polarity, charge, solubility, hydrophobicity, hydrophilicity and/or the amphipathic nature of the residues as long as the endogenous function is retained. For example, negatively charged amino acids include aspartic acid and glutamic acid; positively charged amino acids include lysine and arginine; and amino acids with uncharged polar head groups having similar hydrophilicity values include asparagine, glutamine, serine, threonine and tyrosine.

Conservative substitutions may be made, for example according to the table below. Amino acids in the same block in the second column and preferably in the same line in the third column may be substituted for each other:

| | | |
|---|---|---|
| ALIPHATIC | Non-polar | G A P |
| | | I L V |
| | Polar - uncharged | C S T M |
| | | N Q |
| | Polar - charged | D E |
| | | K R H |
| AROMATIC | | F W Y |

The derivative may be a homolog or variant. The term “homologue” or “variant” as used herein means an entity having a certain homology with the wild type amino acid sequence and the wild type nucleotide sequence. The term “homology” can be equated with “identity”.

A homologous or variant sequence may include an amino acid sequence or a nucleotide sequence which may be at least 50%, 55%, 65%, 75%, 85% or 90% identical, preferably at least 95% or 97% or 99% identical to the subject sequence. Typically, the homologues will have similar chemical properties/functions e.g. comprise the same binding sites etc. as the subject amino acid sequence or the amino acid sequence encoded by the subject nucleotide sequence. Although homology can also be considered in terms of similarity (i.e. amino acid residues having similar chemical properties/functions), in the context of the present invention it is preferred to express homology in terms of sequence identity.

Homology comparisons can be conducted by eye or, more usually, with the aid of readily available sequence comparison programs. These commercially available computer programs can calculate percentage homology or identity between two or more sequences.

Percentage homology may be calculated over contiguous sequences, i.e. one sequence is aligned with the other sequence and each amino acid in one sequence is directly compared with the corresponding amino acid in the other sequence, one residue at a time. This is called an “ungapped” alignment. Typically, such ungapped alignments are performed only over a relatively short number of residues.

Although this is a very simple and consistent method, it fails to take into consideration that, for example, in an otherwise identical pair of sequences, one insertion or deletion in the nucleotide sequence may cause the following codons to be put out of alignment, thus potentially resulting in a large reduction in percent homology when a global alignment is performed. Consequently, most sequence comparison methods are designed to produce optimal alignments that take into consideration possible insertions and deletions without penalising unduly the overall homology score. This is achieved by inserting “gaps” in the sequence alignment to try to maximise local homology.

However, these more complex methods assign “gap penalties” to each gap that occurs in the alignment so that, for the same number of identical amino acids, a sequence alignment with as few gaps as possible, reflecting higher relatedness between the two compared sequences, will achieve a higher score than one with many gaps. “Affine gap costs” are typically used that charge a relatively high cost for the existence of a gap and a smaller penalty for each subsequent residue in the gap. This is the most commonly used gap scoring system. High gap penalties will of course produce optimised alignments with fewer gaps. Most alignment programs allow the gap penalties to be modified. However, it is preferred to use the default values when using such software for sequence comparisons. For example when using the GCG Wisconsin Bestfit package the default gap penalty for amino acid sequences is −12 for a gap and −4 for each extension.

Calculation of maximum percentage homology therefore firstly requires the production of an optimal alignment, taking into consideration gap penalties. A suitable computer program for carrying out such an alignment is the GCG Wisconsin Bestfit package (University of Wisconsin, U.S.A.; Devereux et al. (1984) Nucleic Acids Res. 12: 387). Examples of other software that can perform sequence comparisons include, but are not limited to, the BLAST package (see Ausubel et al. (1999) ibid—Ch. 18), FASTA (Atschul et al. (1990) J. Mol. Biol. 403-410) and the GENEWORKS suite of comparison tools. Both BLAST and FASTA are available for offline and online searching (see Ausubel et al. (1999) ibid, pages 7-58 to 7-60). However, for some applications, it is preferred to use the GCG Bestfit program. Another tool, called BLAST 2 Sequences is also available for comparing protein and nucleotide sequences (see FEMS Microbiol. Lett. (1999) 174: 247-50; FEMS Microbiol. Lett. (1999) 177: 187-8).

Although the final percentage homology can be measured in terms of identity, the alignment process itself is typically not based on an all-or-nothing pair comparison. Instead, a scaled similarity score matrix is generally used that assigns scores to each pairwise comparison based on chemical similarity or evolutionary distance. An example of such a matrix commonly used is the BLOSUM62 matrix—the default matrix for the BLAST suite of programs. GCG Wisconsin programs generally use either the public default values or a custom symbol comparison table if supplied (see the user manual for further details). For some applications, it is preferred to use the public default values for the GCG package, or in the case of other software, the default matrix, such as BLOSUM62. Suitably, the percentage identity is determined across the entirety of the reference and/or the query sequence.

Once the software has produced an optimal alignment, it is possible to calculate percentage homology, preferably percentage sequence identity. The software typically does this as part of the sequence comparison and generates a numerical result.

“Fragments” typically refers to a selected region of the polypeptide or polynucleotide that is of interest functionally. “Fragment” thus refers to an amino acid or nucleic acid sequence that is a portion of a full-length polypeptide or polynucleotide.

Such derivatives, variants and fragments may be prepared using standard recombinant DNA techniques such as site-directed mutagenesis. Where insertions are to be made, synthetic DNA encoding the insertion together with 5′ and 3′ flanking regions corresponding to the naturally-occurring sequence either side of the insertion site may be made. The flanking regions will contain convenient restriction sites corresponding to sites in the naturally-occurring sequence so that the sequence may be cut with the appropriate enzyme(s) and the synthetic DNA ligated into the cut. The DNA is then expressed in accordance with the invention to make the encoded protein. These methods are only illustrative of the numerous standard techniques known in the art for manipulation of DNA sequences and other known techniques may also be used.

Pharmaceutical Composition

There is also provided a pharmaceutical composition comprising an engineered Treg, or CAR, of the invention.

A pharmaceutical composition is a composition that comprises or consists of a therapeutically effective amount of a pharmaceutically active agent i.e. the Treg. It preferably includes a pharmaceutically acceptable carrier, diluent or excipient (including combinations thereof).

By “pharmaceutically acceptable” is included that the formulation is sterile and pyrogen free. The carrier, diluent, and/or excipient must be “acceptable” in the sense of being compatible with the Treg and not deleterious to the recipients thereof. Typically, the carriers, diluents, and excipients will be saline or infusion media which will be sterile and pyrogen free, however, other acceptable carriers, diluents, and excipients may be used.

Acceptable carriers, diluents, and excipients for therapeutic use are well known in the pharmaceutical art. The choice of pharmaceutical carrier, excipient or diluent can be selected with regard to the intended route of administration and standard pharmaceutical practice. The pharmaceutical compositions may comprise as—or in addition to—the carrier, excipient or diluent any suitable binder(s), lubricant(s), suspending agent(s), coating agent(s) or solubilising agent(s).

Examples of pharmaceutically acceptable carriers include, for example, water, salt solutions, alcohol, silicone, waxes, petroleum jelly, vegetable oils, polyethylene glycols, propylene glycol, liposomes, sugars, gelatin, lactose, amylose, magnesium stearate, talc, surfactants, silicic acid, viscous paraffin, perfume oil, fatty acid monoglycerides and diglycerides, petroethral fatty acid esters, hydroxymethyl-cellulose, polyvinylpyrrolidone, and the like.

The Tregs or pharmaceutical compositions according to the present invention may be administered in a manner appropriate for treating and/or preventing the disease described herein. The quantity and frequency of administration will be determined by such factors as the condition of the subject, and the type and severity of the subjects's disease, although appropriate dosages may be determined by clinical trials. The pharmaceutical composition may be formulated accordingly.

The Treg or pharmaceutical composition as described herein can be administered parenterally, for example, intravenously, or they may be administered by infusion techniques. The Treg or pharmaceutical composition may be administered in the form of a sterile aqueous solution which may contain other substances, for example, enough salts or glucose to make the solution isotonic with blood. The aqueous solution may be suitably buffered (preferably to a pH of from 3 to 9). The pharmaceutical composition may be formulated accordingly. The preparation of suitable parenteral formulations under sterile conditions is readily accomplished by standard pharmaceutical techniques well-known to those skilled in the art.

The pharmaceutical compositions may comprise Tregs of the invention in infusion media, for example sterile isotonic solution. The pharmaceutical composition may be enclosed in ampoules, disposable syringes or multiple dose vials made of glass or plastic.

The Treg or pharmaceutical composition may be administered in a single or in multiple doses. Particularly, the Treg or pharmaceutical composition may be administered in a single, one off dose. The pharmaceutical composition may be formulated accordingly.

Depending upon the disease and subject to be treated, as well as the route of administration, the Treg or pharmaceutical composition may be administered at varying doses (e.g. measured in cells/kg or cells/subject). The physician in any event will determine the actual dosage which will be most suitable for any individual subject and it will vary with the age, weight and response of the particular subject. Typically, however, for Tregs of the invention, doses of 5×10⁷ to 3×10⁹ cells, or 10⁸ to 2×10⁹ cells per subject may be administered.

The Treg may be appropriately modified for use in a pharmaceutical composition. For example, Tregs may be cryopreserved and thawed at an appropriate time, before being infused into a subject.

The pharmaceutical composition may further comprise one or more other therapeutic agents, such as lympho-depletive agents (e.g. thymoglobulin, campath-1H, anti-CD2 antibodies, anti-CD3 antibodies, anti-CD20 antibodies, cyclophosphamide, fludarabine), inhibitors of mTOR (e.g. sirolimus, everolimus), drugs inhibiting costimulatory pathways (e.g. anti-CD40/CD40L, CTAL4lg), and/or drugs inhibiting specific cytokines (IL-6, IL-17, TNFalpha, IL18).

The invention further includes the use of kits comprising the Treg and/or pharmaceutical composition of the present invention. Preferably said kits are for use in the methods and uses as described herein, e.g., the therapeutic methods as described herein. Preferably said kits comprise instructions for use of the kit components.

Methods for Treating and/or Preventing Disease

The engineered Tregs may be administered to a subject having an existing disease or condition in order to lessen, reduce, or improve at least one symptom associated with the disease and/or to slow down, reduce, or block the progression of the disease.

For example, the engineered Tregs may be administered to a subject with a liver disease (e.g. liver transplant rejection, liver GvHD, autoimmune liver disease, liver inflammation, liver failure) in order to lessen, reduce, or improve at least one symptom of liver disease such as jaundice, dark urine, itching, abdominal swelling or tenderness, fatigue, nausea or vomiting, and/or loss of appetite. The at least one symptom may be lessened, reduced, or improved by at least 10%, at least 20%, at least 30%, at least 40%, or at least 50%, or the at least one symptom may be completely alleviated.

The engineered Tregs may be administered to a subject with a liver disease (e.g. liver transplant rejection, liver GvHD, autoimmune liver disease, liver inflammation, liver failure) in order to slow down, reduce, or block the progression of the liver disease. The progression of the disease may be slowed down, reduced, or blocked by at least 10%, at least 20%, at least 30%, at least 40%, or at least 50% compared to a subject in which the engineered Tregs are not administered, or progression of the disease may be completely stopped.

Alternatively, the engineered Tregs may be administered to a subject who has not yet contracted the disease and/or who is not showing any symptoms of the disease, to prevent the disease or to reduce or prevent development of at least one symptom associated with the disease. The subject may have a predisposition for, or be though to be at risk of developing, the disease.

For example, the engineered Tregs may be administered to a subject who has not yet contracted and/or who is not showing any symptoms of liver disease (e.g. liver transplant rejection, liver GvHD, autoimmune liver disease, liver inflammation, liver failure) in order to reduce or prevent at least one symptom of liver disease such as jaundice, dark urine, itching, abdominal swelling or tenderness, fatigue, nausea or vomiting, and/or loss of appetite. The at least one symptom may be reduced by at least 10%, at least 20%, at least 30%, at least 40%, or at least 50% compared to a subject in which the engineered Tregs are not administered, or the at least one symptom may be completely prevented.

The engineered Tregs may be administered to a subject who has not yet contracted and/or who is not showing any symptoms of liver disease (e.g. liver transplant rejection, liver GvHD, autoimmune liver disease, liver inflammation, liver failure) in order to prevent the liver disease. The liver disease may be impaired by at least 10%, at least 20%, at least 30%, at least 40%, or at least 50% compared to a subject in which the engineered Tregs are not administered, or the liver disease may be completely prevented.

Liver disease includes conditions such as liver transplant rejection, liver GvHD, autoimmune liver disease, liver inflammation and liver failure, and particularly includes liver diseases associated with an undesired increased immune response in a subject which may result in liver damage or destruction. In a particular embodiment, liver disease may not, for example, include liver cancer, and more particularly may not include hepatocellular carcinoma (HCC). Liver disease thus preferably includes conditions which may be treated or prevented by reduction of the immune response (e.g. by a reduction of at least 10, 20, 30, 40, 50, 60, 70, 80, or 90%), particularly in or near the liver.

Suitably, the therapeutic methods of the invention may comprise the step of administering an engineered Treg according to the invention, or obtainable (e.g. obtained) by a method according to the present invention, or a polynucleotide or vector as defined herein (for example in a pharmaceutical composition as described herein) to a subject.

Suitably, the present methods for treating and/or preventing a disease may comprise administering an engineered Treg according to the present invention (for example in a pharmaceutical composition as described herein) to a subject.

The method may involve the steps of:
(i) isolating a cell-containing sample or providing a cell-containing sample;
(ii) introducing a polynucleotide or a vector as defined herein to the cells; and
(iii) administering the cells from (ii) to a subject.

Suitably, the cell is a Treg as defined herein.

Suitably, an enriched Treg population may be isolated from and/or generated from the cell-containing sample prior to and/or after step (ii) of the method. For example, isolation and/or generation may be performed prior to and/or after step (ii) to isolate and/or generate an enriched Treg sample. Enrichment may be performed after step (ii) to enrich for cells and/or Tregs comprising the CAR, the polynucleotide, and/or the vector of the present invention.

Suitably, the polynucleotide or vector may be introduced by transduction and/or transfection.

Suitably, the cell may be autologous and/or allogenic.

Suitably, the engineered Treg may be administered in combination with one or more other therapeutic agents, such as lympho-depletive agents (e.g. thymoglobulin, campath-1H, anti-CD2 antibodies, anti-CD3 antibodies, anti-CD20 antibodies, cyclophosphamide, fludarabine), inhibitors of mTOR (e.g. sirolimus, everolimus), drugs inhibiting costimulatory pathways (e.g. anti-CD40/CD40L, CTAL4lg), and/or drugs inhibiting specific cytokines (IL-6, IL-17, TNFalpha, IL18). The engineered Treg may be administered simultaneously with or sequentially with (i.e. prior to or after) the one or more other therapeutic agents.

Liver Transplant

Liver transplantation is the only curative treatment option currently available for patients with end-stage liver disease. Despite recent advances in immunosuppressive agents, acute allograft rejection remains a common complication of liver transplantation, with the incidence ranging from 20% to 40% of liver transplants. In most cases, rejection occurs within the first month following the liver transplant. Early rejection episodes do not significantly impair long-term graft success or patient outcomes. In contrast, late-onset allograft rejection (>3-6 months following liver transplant) is associated with poor graft survival (Dogan, N., et al., 2018. Journal of International Medical Research, 46(9), pp. 3979-3990).

The present invention provides a method of inducing tolerance to a liver transplant, which comprises the step of administering an engineered Treg or a pharmaceutical composition of the invention to a subject. Suitably, the subject is mammal, preferably human.

As used herein, “inducing tolerance to a liver transplant” refers to inducing tolerance to a transplanted liver in a recipient. In other words, inducing tolerance to a liver transplant means to reduce the level of a recipient's immune response to a donor transplant organ. Inducing tolerance to a transplanted liver may decrease the incidence of rejection, reduce the amount of immunosuppressive drugs that the transplant patient requires, or may enable the discontinuation of immunosuppressive drugs.

The present invention also provides a method of treating and/or preventing liver transplant rejection, which comprises the step of administering an engineered Treg or a pharmaceutical composition of the invention to a subject. Suitably, the subject is mammal, preferably human. Suitably, treating and/or preventing liver transplant rejection may refer to reducing the amount of immunosuppressive drugs that a liver transplant recipient requires, or may enable the discontinuation of immunosuppressive drugs.

In one embodiment, the subject is a liver transplant recipient undergoing immunosuppression therapy.

In one embodiment, the present invention promotes liver tissue repair and/or liver regeneration preferably in addition to inducing tolerance to a liver transplant or treating and/or preventing liver transplant rejection.

Graft-Versus-Host Disease

The present invention provides a method of treating and/or preventing liver graft-versus-host disease (GvHD), which comprises the step of administering an engineered Treg or a pharmaceutical composition of the invention to a subject. The subject may be a liver transplant recipient. Suitably, the subject is mammal, preferably human.

GvHD is a common complication following the receipt of transplanted tissue from a genetically different person. GvHD is commonly associated with stem cell transplants such as those that occur with bone marrow transplants. GvHD also applies to other forms of transplanted tissues such as liver transplants. White blood cells of the donor's immune system which remain within the donated tissue (the graft) recognize the recipient (the host) as foreign (non-self). The white blood cells present within the transplanted tissue then attack the recipient's body's cells, which leads to GvHD. In the classical sense, acute graft-versus-host-disease is characterized by selective damage to the liver, skin, mucosa, and the gastrointestinal tract. Accordingly, the subject may have liver damage, i.e. liver GvHD.

In some embodiments the subject is a transplant recipient wherein the transplant is selected from liver, kidney, heart, lung, pancreas, intestine, stomach, bone marrow, vascularized composite tissue graft and skin transplant. Preferably the subject is a liver transplant recipient.

In one embodiment, the subject is a liver transplant recipient undergoing immunosuppression therapy.

In one embodiment, the present invention promotes liver tissue repair and/or liver regeneration in addition to treating and/or preventing liver GvHD.

Autoimmune Liver Disease

The present invention provides a method of treating and/or preventing an autoimmune liver disease, which comprises the step of administering an engineered Treg or a pharmaceutical composition of the invention to a subject. Suitably, the subject is mammal, preferably human.

Suitably, the autoimmune liver disease is a chronic autoimmune liver disease.

The autoimmune liver disease may be selected from one or more of autoimmune hepatitis, primary biliary cholangitis and/or (primary) sclerosing cholangitis. Autoimmune liver diseases are chronic, slowly progressive, inflammatory liver diseases that may have overlapping features (Decock, S., McGee, P. and Hirschfield, G. M., 2009. Bmj, 339, p. b3305).

Autoimmune hepatitis is usually a relapsing immune-mediated hepatitis. Patients present clinically with arthralgias and fatigue if symptomatic, and a third of patients present with cirrhosis. Raised liver enzymes (transaminases) characterise initial laboratory abnormalities. (Decock, S., McGee, P. and Hirschfield, G. M., 2009. Bmj, 339, p. b3305). Autoimmune hepatitis can also present as an acute disease that, if left untreated, leads to liver failure and death.

Primary biliary cholangitis is a slowly progressive, chronic cholestatic disease and is characterised by small duct granulomatous cholangitis and biochemical cholestasis (raised alkaline phosphatase). Currently 60% of patients diagnosed with primary biliary cholangitis have no symptoms, and most have non-cirrhotic disease. When symptoms are present, fatigue, pruritus, and right upper quadrant discomfort are common, but do not indicate severity of disease. Even in the absence of symptoms, however, patients with primary biliary cholangitis have a significantly decreased long-term survival as compared to the general population (Decock, S., McGee, P. and Hirschfield, G. M., 2009. Bmj, 339, p. b3305).

Primary sclerosing cholangitis is a chronic cholestatic liver disease characterised by fibrosing inflammatory destruction of the intrahepatic and/or extrahepatic biliary tree. There are currently no treatments for primary sclerosing cholangitis. Once symptoms are present, there is about a 50% chance of need for transplantation and 10% risk of cholangiocarcinoma over 10 years. (Decock, S., McGee, P. and Hirschfield, G. M., 2009. Bmj, 339, p. b3305).

In one embodiment, the present invention promotes liver tissue repair and/or liver regeneration in addition to treating and/or preventing autoimmune liver disease.

Liver Inflammation

The present invention provides a method of treating and/or preventing an inflammatory liver disorder, which comprises the step of administering an engineered Treg or a pharmaceutical composition of the invention to a subject. Suitably, the subject is mammal, preferably human.

Inflammation of the liver tissue is also known as hepatitis. Hepatitis may be acute or chronic. Acute hepatitis can sometimes resolve on its own, progress to chronic hepatitis, or rarely result in acute liver failure. Over time the chronic form may progress to scarring of the liver (cirrhosis), chronic liver failure, and/or liver cancer. Signs and symptoms of liver cirrhosis include jaundice, ascites (fluid accumulation in the abdominal cavity), fatigue and hepatic encephalopathy (brain dysfunction due to liver failure).

Causes of hepatitis can be divided into the following major categories: infectious, metabolic, alcoholic, ischemic, autoimmune, genetic. Thus, in some embodiment the hepatitis is selected from infectious hepatitis, metabolic hepatitis, alcoholic hepatitis, ischemic hepatitis, autoimmune hepatitis, and genetic hepatitis.

Infectious hepatitis includes viral hepatitis, parasitic hepatitis, and bacterial hepatitis.

Viral hepatitis is liver inflammation due to a viral infection. It may present in acute form as a recent infection with relatively rapid onset, or in chronic form. The most common causes of viral hepatitis are the five unrelated hepatotropic viruses hepatitis A, B, C, D, and E. Other viruses can also cause liver inflammation, including cytomegalovirus, Epstein-Barr virus, and yellow fever. There also have been scores of recorded cases of viral hepatitis caused by herpes simplex virus.

Parasitic hepatitis is liver inflammation due to a parasitic infection. Of the protozoans, Trypanosoma cruzi, Leishmania species, and the malaria-causing Plasmodium species all can cause liver inflammation. Of the worms, dog tapeworm, infects the liver and forms characteristic hepatic hydatid cysts. The liver flukes Fasciola hepatica and Clonorchis sinensis live in the bile ducts and cause progressive hepatitis and liver fibrosis.

Bacterial hepatitis is liver inflammation due to a bacterial infection. Acute hepatitis is caused by Neisseria meningitidis, Neisseria gonorrhoeae, Bartonella henselae, Borrelia burgdorferi, salmonella species, brucella species and campylobacter species. Chronic or granulomatous hepatitis is seen with infection from mycobacteria species, Tropheryma whipplei, Treponema pallidum, Coxiella burnetii, and rickettsia species.

Alcoholic hepatitis is inflammation of the liver due to excessive intake of alcohol. Alcoholic hepatitis can have a chronic course that can lead to cirrhosis, liver failure and/or cancer, or an acute presentation. The severe cases of acute alcoholic hepatitis have a 50% 3 month mortality. Many chemical agents, including medications, industrial toxins, and herbal and dietary supplements, can also cause toxic hepatitis.

Non-alcoholic steatohepatitis, the most frequent form of metabolic hepatitis, is within the spectrum of non-alcoholic fatty liver disease (NAFLD). Non-alcoholic fatty liver disease occurs in people with little or no history of alcohol use, and is instead strongly associated with metabolic syndrome, obesity, insulin resistance and diabetes, and hypertriglyceridemia. Non-alcoholic fatty liver disease can result in non-alcoholic steatohepatitis. Steatohepatitis is a type of fatty liver disease, characterized by inflammation of the liver with concurrent fat accumulation in liver, which can lead to cirrhosis, liver failure and/or liver cancer.

Ischemic hepatitis also known as ischemic hepatopathy or shock liver, is a condition defined as an acute liver injury caused by insufficient blood flow (and consequently insufficient oxygen delivery) to the liver.

Genetic causes of hepatitis include alpha-1-antitrypsin deficiency, hemochromatosis, and Wilson's disease.

In some embodiments the liver inflammation has no identifiable cause.

In one embodiment, the present invention promotes liver tissue repair and/or liver regeneration in addition to treating and/or preventing an inflammatory liver disorder.

Liver Repair or Regeneration

The present invention provides a method of promoting liver tissue repair and/or liver regeneration, which comprises the step of administering an engineered Treg or a pharmaceutical composition of the invention to a subject. Suitably, the subject is mammal, preferably human. The subject may have liver cirrhosis, acute liver failure or acute-on-chronic liver failure.

As used herein, “liver regeneration” may refer to the recreation of liver architecture and function following damage (e.g. acute damage), without leaving a scar (Cordero-Espinoza, L. and Huch, M., 2018. The Journal of clinical investigation, 128(1), pp. 85-96). For example, liver regeneration may result in restoration of at least 25%, at least 50%, at least 60%, at least 70%, at least 80%, at least 90%, or 100% of the original liver mass. The method of the present invention may reduce the time until maximal liver mass is achieved, for example the time may be reduced by at least 10%, at least 20%, at least 30%, at least 40%, or by at least 50% compared to a subject in which the engineered Treg or pharmaceutical composition are not administered.

As used herein, “liver tissue repair” may refer to the recreation of liver architecture and function following damage (e.g. chronic damage), with scarring (i.e. fibrosis) (Cordero-Espinoza, L. and Huch, M., 2018. The Journal of clinical investigation, 128(1), pp. 85-96). This may be characterised by replacement of functional tissue parenchyma with a meshwork of extracellular matrix (ECM). The liver architecture may be altered and optimal function may be hindered. For example, liver repair may result in restoration of at least 10%, at least 20%, at least 30%, at least 40%, or at least 50% of the original liver function. The method of the present invention may reduce the time until maximal liver function is reached, for example the time may be reduced by at least 10%, at least 20%, at least 30%, at least 40%, or by at least 50% compared to a subject in which the engineered Treg or pharmaceutical composition are not administered.

The engineered Treg of the invention may express genes involved in liver regeneration and tissue repair, thus promoting robust liver repair and/or regeneration.

Although adult hepatocytes are long lived and normally do not undergo cell division, they maintain the ability to proliferate in response to inflammatory damage or following partial hepatectomy. This ability is most clearly shown by the two-thirds partial-hepatectomy model in rodents. In this model, two thirds of the liver is surgically removed, and the remaining liver enlarges until the original liver mass is restored—approximately 1 week after surgery—after which the regenerative process stops. In humans, liver regeneration occurs most frequently after liver damage by ischaemia or hepatitis. (Taub, R., 2004. Nature reviews Molecular cell biology, 5(10), p. 836-847). A very similar phenomenon occurs in humans who undergo a partial hepatectomy as treatment for liver tumours or during living donor liver transplantation. In addition, in humans liver regeneration occurs following damage to the hepatocytes due to ischemia, toxics, and acute or chronic hepatitis.

Suitably, the liver may be injured and/or damaged by hepatitis. In some embodiments, in addition to promoting liver tissue repair and/or liver regeneration, the engineered Treg of the invention further treats the hepatitis. The hepatitis may be selected from infectious hepatitis, metabolic hepatitis, alcoholic hepatitis, ischemic hepatitis, autoimmune hepatitis, and genetic hepatitis. In one embodiment, hepatitis may not be fulminant or chronic hepatitis.

Due to liver regeneration, it has become possible to use partial livers from living donors for transplantation, thereby increasing the number of organs that are available for transplantation. Increasing numbers of liver transplants are being undertaken using living, related-donor tissue and small-for-size transplant organs, for which successful transplantation requires at least some liver regeneration and repair. (Taub, R., 2004. Nature reviews Molecular cell biology, 5(10), p. 836-847).

Thus, the subject may be a liver transplant recipient or a patient who is undergoing a partial hepatectomy (e.g. as a living donor for liver transplantation or due to the presence of a liver tumour that requires surgical resection). The liver may be a transplanted liver.

Humans with certain hepatic conditions, including cirrhosis (fibrosis of the liver), steatosis (fatty liver), and even those conditions that are due to old age, have impaired liver regeneration that results in increased morbidity and mortality in response to liver injury or damage. (Taub, R., 2004. Nature reviews Molecular cell biology, 5(10), p. 836-847).

Thus, the subject may have impaired liver regeneration, preferably due to one or more of acute liver failure, cirrhosis, acute-on-chronic liver failure, hepatitis, steatosis, steatohepatitis and old age.

In one embodiment, the present invention treats and/or prevents immune-mediated damage in addition to promoting liver tissue repair and/or liver regeneration. For example, the present invention may also induce tolerance to a liver transplant in the subject, or treat and/or prevent liver transplant rejection, liver graft-versus-host disease (GvHD), an autoimmune liver disease, or an inflammatory liver disorder in the subject.

Preferred CAR Tregs for use in these embodiments for repair and/or regeneration (and other embodiments described herein wherein repair and/or regeneration is involved) are capable of increasing or stimulating albumin production, for example are capable of increasing albumin concentrations or levels and/or stimulating albumin production, in the liver, for example in liver cells or liver tissue, for example in hepatocytes.

Thus, a yet further aspect of the invention provides an engineered regulatory T cell (Treg) comprising a chimeric antigen receptor (CAR), or a pharmaceutical composition comprising the engineered Treg, for use in increasing or stimulating albumin production, or for use in increasing albumin levels or stimulating albumin production, in liver tissue or liver cells in a subject, wherein the CAR comprises a liver-specific antigen recognition domain.

Viewed alternatively, this aspect of the invention provides a method of increasing or stimulating albumin production, or a method of increasing albumin levels, in liver tissue or liver cells in a subject, which comprises the step of administering to the subject an engineered regulatory T cell (Treg) comprising a chimeric antigen receptor (CAR), or a pharmaceutical composition comprising the engineered Treg, wherein the CAR comprises a liver-specific antigen recognition domain.

Viewed alternatively, this aspect of the invention provides the use of an engineered regulatory T cell (Treg) comprising a chimeric antigen receptor (CAR), or a pharmaceutical composition comprising the engineered Treg, in the manufacture of a medicament or composition for increasing or stimulating albumin production, or for increasing albumin levels, in liver tissue or liver cells in a subject, wherein the CAR comprises a liver-specific antigen recognition domain.

Appropriate and preferred liver-specific antigen recognition domains for use in such aspects where albumin levels are increased are described elsewhere herein, for example an antigen recognition domain which binds or specifically binds to ASGR. Other preferred embodiments for this aspect are also described elsewhere herein.

Hepatocytes typically make up the majority of the liver mass and albumin levels or albumin concentration within hepatocytes, within the supernatant when hepatocytes or liver tissue samples are cultured in vitro or in the blood of patients, is a marker of liver cell, e.g. hepatocyte, differentiation, function and viability. Thus, determining the effect of Tregs comprising a chimeric antigen receptor (CAR) on albumin levels within such cells provides a measurement or indication of the ability of the CAR Tregs to mediate trophic (e.g. trophic signals that promote hepatocyte function and/or viability) and/or cytoprotective effects on liver cells or liver tissue in response to antigenic stimulation.

Tregs comprising a chimeric antigen receptor (CAR) which are capable of increasing or stimulating albumin production, or of increasing albumin levels or concentration, can be identified using any suitable assay. Suitable assays would be well known in the art. For example, in vitro assays using appropriate liver cells and measuring the levels of albumin in these cells or in the supernatant from these cells (for example to assess the levels of secreted albumin) by routine techniques (e.g. by ELISA), could readily be used. Levels could conveniently be measured and compared in the presence and absence of an appropriate CAR Treg. Hepatocytes, in particular human hepatocytes, for example primary human hepatocytes, are particularly appropriate. An exemplary assay is shown in Example 14. As described above, such CAR Tregs can in turn have the ability to mediate trophic and/or cytoprotective effects on liver cells in response to antigenic stimulation, thereby making them particularly appropriate for use in aspects of the invention involving liver repair and/or regeneration. Albumin levels or concentration within a patient after treatment with an engineered Treg as defined herein, can be measured via a blood sample using routine techniques as discussed above.

Reference to an increase or stimulation (or equivalent terms) in albumin production or in albumin levels or concentrations in liver cells or liver tissue as referred to herein, includes any measurable increase or enhancement or improvement when compared with an appropriate control. Preferably such increases, etc., are significant increases, preferably clinically significant or statistically significant increases, for example with a probability value of <0.05, when compared to an appropriate control level or value.

Appropriate controls would readily be identified by a person skilled in the art and might include for example production, levels or concentrations of albumin observed in the presence of a CAR Treg of the invention (for example a transduced CAR Treg of the invention) in comparison to the absence of said CAR Treg (e.g. compared to an untreated sample, e.g. compared to appropriate control medium only), or might include for example production, levels or concentrations of albumin observed in the presence of a CAR Treg of the invention (for example a transduced CAR Treg of the invention) in comparison to the presence of a non-transduced Treg (e.g. an activated or non-activated/resting non-transduced Treg).

In such embodiments where increased albumin production, levels or concentrations in liver cells are observed with a CAR Treg of the invention (for example a transduced CAR Treg of the invention), it is preferred that an improvement or increase in albumin levels or concentrations of at least (or up to) 1.5 fold, 2 fold, 3 fold, 4, fold, 5 fold, 7 fold, 10 fold, 15 fold, 20 fold, 25 fold, 30 fold, 35 fold, 40 fold, 45 fold, or 50 fold, is observed. For example, an improvement or increase in albumin levels or concentrations of at least (or up to) 5 fold, 7 fold, 10 fold, 15 fold, 20 fold, 25 fold, 30 fold, 35 fold, 40 fold, 45 fold, or 50 fold, may be observed with a preferred CAR Treg of the invention in comparison to the levels or concentrations observed in the absence of a CAR Treg of the invention, e.g. when compared with an untreated sample or appropriate control medium only. For example, an improvement or increase in albumin levels of at least (or up to) 1.5 fold, 2 fold, 3 fold, 4, fold, 5 fold, 7 fold, or 10 fold may be observed with a preferred CAR Treg of the invention in comparison to the levels observed with a non-transduced or non-transfected Treg, e.g. when compared with non-transduced activated or non-activated/resting Tregs.

Liver Fibrosis, Cirrhosis, Acute Liver Failure and Acute-on-Chronic Liver Failure

The present invention provides a method of treating and/or preventing liver fibrosis, liver cirrhosis, acute liver failure or acute-on-chronic liver failure.

Improvement of liver fibrosis, liver cirrhosis, acute liver failure or acute-on-chronic liver failure will typically require treating and/or preventing immune-mediated damage of the liver and promoting liver regeneration.

Liver fibrosis is one of the leading causes of mortality because it changes the architecture of certain organs and disrupts normal function. Liver fibrosis is a histological consequence of the wound-healing process resulting from chronic liver diseases such as viral hepatitis, alcoholic liver disease, non-alcoholic fatty liver disease, and other liver disorders. Deposition of excess extracellular matrix (ECM) that is rich in fibril-forming collagens is a typical finding of liver fibrosis. The excess deposition of the ECM changes the normal architecture of the liver resulting in pathophysiologic damage to the organ (Suk, K. T. and Kim, D. J., 2015. World journal of hepatology, 7(3), p. 607).

Liver cirrhosis is defined as an advanced stage of liver fibrosis with distortion of the hepatic vasculature and architecture. Histologically, regenerative nodules with fibrous tissues form in response to chronic injury and lead to liver cirrhosis. Consequently, disruption of the liver architecture due to liver fibrosis and/or cirrhosis causes hemodynamic instability and portal hypertension (Suk, K. T. and Kim, D. J., 2015. World journal of hepatology, 7(3), p. 607).

Acute liver failure is defined herein as the rapid development of hepatocellular dysfunction, specifically coagulopathy and encephalopathy in a patient without known prior liver disease. For example, “acute hepatic failure” may be defined as the development of encephalopathy within 26 weeks of the onset of any hepatic symptoms. This may be sub-divided into “fulminant hepatic failure”, which requires onset of encephalopathy within 8 weeks, and “subfulminant”, which describes onset of encephalopathy after 8 weeks but before 26 weeks. Another scheme defines “hyperacute” as onset within 7 days, “acute” as onset between 7 and 28 days, and “subacute” as onset between 28 days and 24 weeks.

Acute-on-chronic liver failure is characterised by acute decompensation of chronic liver disease associated with organ failures and high short-term mortality. Alcohol and chronic viral hepatitis are the most common underlying liver diseases (Hernaez, R., et al., 2017. Gut, 66(3), pp. 541-553).

Polynucleotides

The present invention provides a polynucleotide encoding the CAR of the invention.

Polynucleotides of the invention may comprise DNA or RNA. They may be single-stranded or double-stranded. It will be understood by a skilled person that numerous different polynucleotides can encode the same polypeptide as a result of the degeneracy of the genetic code. In addition, it is to be understood that the skilled person may, using routine techniques, make nucleotide substitutions that do not affect the polypeptide sequence encoded by the polynucleotides of the invention to reflect the codon usage of any particular host organism in which the polypeptides of the invention are to be expressed.

The polynucleotides may be modified by any method available in the art. Such modifications may be carried out in order to enhance the in vivo activity or lifespan of the polynucleotides of the invention.

Polynucleotides such as DNA polynucleotides may be produced recombinantly, synthetically or by any means available to those of skill in the art. They may also be cloned by standard techniques.

Longer polynucleotides will generally be produced using recombinant means, for example using polymerase chain reaction (PCR) cloning techniques. This will involve making a pair of primers (e.g. of about 15 to 30 nucleotides) flanking the target sequence which it is desired to clone, bringing the primers into contact with mRNA or cDNA obtained from an animal or human cell, performing a polymerase chain reaction under conditions which bring about amplification of the desired region, isolating the amplified fragment (e.g. by purifying the reaction mixture with an agarose gel) and recovering the amplified DNA. The primers may be designed to contain suitable restriction enzyme recognition sites so that the amplified DNA can be cloned into a suitable vector.

The polynucleotides used in the present invention may be codon-optimised. Codon optimisation has previously been described in WO 1999/41397 and WO 2001/79518. Different cells differ in their usage of particular codons. This codon bias corresponds to a bias in the relative abundance of particular tRNAs in the cell type. By altering the codons in the sequence so that they are tailored to match with the relative abundance of corresponding tRNAs, it is possible to increase expression. By the same token, it is possible to decrease expression by deliberately choosing codons for which the corresponding tRNAs are known to be rare in the particular cell type. Thus, an additional degree of translational control is available.

Illustrative nucleotide sequences encoding illustrative CARs of the present invention (SEQ ID NOs: 149-152 or 167-169) are shown below. The polynucleotide encoding the CAR of the invention may comprise a sequence which has at least 70, 80, 85, 90, 95, 97, 98, 99% or 100% identity to one or more of SEQ ID NOs: 153-156 or 170-172.

-Illustrative nucleotide sequence encoding illustrative CAR 1: CAR
containing CD8 leader, ASGR1 VH, CD8α hinge, CD28 transmembrane, CD28
cytoplasmic, CD3z cytoplasmic
SEQ ID NO: 153
ATGGCCTTACCAGTGACCGCCTTGCTCCTGCCGCTGGCCTTGCTGCTCCACGCCGCCA
GGCCGGAGGTGCAGCTGTTGGAGTCTGGGGGAGGCTTGGTACAGCCTGGGGGGTCC
CTGCGTCTCTCCTGTGCAGCCTCCGGATTCACCTTTGAGAAGTATGCGATGGCGTGGG
TCCGCCAGGCCCCAGGGAAGGGTCTGGAGTGGGTCTCACGGATTTCGGCGAGGGGTG
TGACGACATACTACGCAGACTCCGTGAAGGGCCGGTTCACCATCTCCCGCGACAATTC
CAAGAACACGCTGTATCTGCAAATGAACAGCCTGCGTGCTGAGGACACCGCGGTATAT
TACTGTGCGAAACATAAGCGGCACGAGCATACTCGTTTTGACTCCTGGGGTCAGGGAA
CCCTGGTCACCGTCTCGAGCACCACGACGCCAGCGCCGCGACCACCAACACCGGCGC
CCACCATCGCGTCGCAGCCCCTGTCCCTGCGCCCAGAGGCGTGCCGGCCAGCGGCG
GGGGGCGCAGTGCACACGAGGGGGCTGGACTTCGCCTGTGATTTCTGGGTGCTGGTG
GTGGTGGGCGGCGTGCTGGCCTGCTACAGCCTGCTGGTGACCGTGGCCTTCATCATC
TTCTGGGTGCGGAGCAAGCGGAGCCGGCTGCTGCACAGCGACTACATGAACATGACC
CCCCGGCGGCCTGGGCCCACCCGCAAGCATTACCAGCCCTATGCCCCACCACGCGAC
TTCGCAGCCTATCGCTCCAGAGTGAAGTTCAGCAGGAGCGCAGACGCCCCCGCGTAC
CAGCAGGGCCAGAACCAGCTCTATAACGAGCTCAATCTAGGACGAAGAGAGGAGTACG
ATGTTTTGGACAAGAGACGTGGCCGGGACCCTGAGATGGGGGGAAAGCCGAGAAGGA
AGAACCCTCAGGAAGGCCTGTACAATGAACTGCAGAAAGATAAGATGGCGGAGGCCTA
CAGTGAGATTGGGATGAAAGGCGAGCGCCGGAGGGGCAAGGGGCACGATGGCCTTTA
CCAGGGTCTCAGTACAGCCACCAAGGACACCTACGACGCCCTTCACATGCAGGCCCTG
CCCCCTCGCTAA
-Illustrative nucleotide sequence encoding illustrative CAR 2: CAR
containing CD8 leader, ASGR1 VH, CD28 hinge with c-Myc tag, CD28
transmembrane, CD28 cytoplasmic, CD3z cytoplasmic
SEQ ID NO: 154
ATGGCCTTACCAGTGACCGCCTTGCTCCTGCCGCTGGCCTTGCTGCTCCACGCCGCCA
GGCCGGAGGTGCAGCTGTTGGAGTCTGGGGGAGGCTTGGTACAGCCTGGGGGGTCC
CTGCGTCTCTCCTGTGCAGCCTCCGGATTCACCTTTGAGAAGTATGCGATGGCGTGGG
TCCGCCAGGCCCCAGGGAAGGGTCTGGAGTGGGTCTCACGGATTTCGGCGAGGGGTG
TGACGACATACTACGCAGACTCCGTGAAGGGCCGGTTCACCATCTCCCGCGACAATTC
CAAGAACACGCTGTATCTGCAAATGAACAGCCTGCGTGCTGAGGACACCGCGGTATAT
TACTGTGCGAAACATAAGCGGCACGAGCATACTCGTTTTGACTCCTGGGGTCAGGGAA
CCCTGGTCACCGTCTCGAGCGCGGCCGCCATCGAGGTGGAGCAGAAGCTGATCAGCG
AGGAGGACCTGCTGGACAACGAGAAGAGCAACGGCACCATCATCCACGTGAAGGGCA
AGCACCTGTGCCCCAGCCCCCTGTTCCCCGGCCCCAGCAAGCCCTTCTGGGTGCTGG
TGGTGGTGGGCGGCGTGCTGGCCTGCTACAGCCTGCTGGTGACCGTGGCCTTCATCA
TCTTCTGGGTGCGGAGCAAGCGGAGCCGGCTGCTGCACAGCGACTACATGAACATGA
CCCCCCGGCGGCCTGGGCCCACCCGCAAGCATTACCAGCCCTATGCCCCACCACGCG
ACTTCGCAGCCTATCGCTCCAGAGTGAAGTTCAGCAGGAGCGCAGACGCCCCCGCGT
ACCAGCAGGGCCAGAACCAGCTCTATAACGAGCTCAATCTAGGACGAAGAGAGGAGTA
CGATGTTTTGGACAAGAGACGTGGCCGGGACCCTGAGATGGGGGGAAAGCCGAGAAG
GAAGAACCCTCAGGAAGGCCTGTACAATGAACTGCAGAAAGATAAGATGGCGGAGGCC
TACAGTGAGATTGGGATGAAAGGCGAGCGCCGGAGGGGCAAGGGGCACGATGGCCTT
TACCAGGGTCTCAGTACAGCCACCAAGGACACCTACGACGCCCTTCACATGCAGGCCC
TGCCCCCTCGCTAA
-Illustrative nucleotide sequence encoding illustrative CAR 3: CAR
containing CD8 leader, ASGR1 VH, CD8α hinge, CD28 transmembrane, CD28
cytoplasmic, CD3z cytoplasmic, FP2A domain, GFP
SEQ ID NO: 155
ATGGCCTTACCAGTGACCGCCTTGCTCCTGCCGCTGGCCTTGCTGCTCCACGCCGCCA
GGCCGGAGGTGCAGCTGTTGGAGTCTGGGGGAGGCTTGGTACAGCCTGGGGGGTCC
CTGCGTCTCTCCTGTGCAGCCTCCGGATTCACCTTTGAGAAGTATGCGATGGCGTGGG
TCCGCCAGGCCCCAGGGAAGGGTCTGGAGTGGGTCTCACGGATTTCGGCGAGGGGTG
TGACGACATACTACGCAGACTCCGTGAAGGGCCGGTTCACCATCTCCCGCGACAATTC
CAAGAACACGCTGTATCTGCAAATGAACAGCCTGCGTGCTGAGGACACCGCGGTATAT
TACTGTGCGAAACATAAGCGGCACGAGCATACTCGTTTTGACTCCTGGGGTCAGGGAA
CCCTGGTCACCGTCTCGAGCACCACGACGCCAGCGCCGCGACCACCAACACCGGCGC
CCACCATCGCGTCGCAGCCCCTGTCCCTGCGCCCAGAGGCGTGCCGGCCAGCGGCG
GGGGGCGCAGTGCACACGAGGGGGCTGGACTTCGCCTGTGATTTCTGGGTGCTGGTG
GTGGTGGGCGGCGTGCTGGCCTGCTACAGCCTGCTGGTGACCGTGGCCTTCATCATC
TTCTGGGTGCGGAGCAAGCGGAGCCGGCTGCTGCACAGCGACTACATGAACATGACC
CCCCGGCGGCCTGGGCCCACCCGCAAGCATTACCAGCCCTATGCCCCACCACGCGAC
TTCGCAGCCTATCGCTCCAGAGTGAAGTTCAGCAGGAGCGCAGACGCCCCCGCGTAC
CAGCAGGGCCAGAACCAGCTCTATAACGAGCTCAATCTAGGACGAAGAGAGGAGTACG
ATGTTTTGGACAAGAGACGTGGCCGGGACCCTGAGATGGGGGGAAAGCCGAGAAGGA
AGAACCCTCAGGAAGGCCTGTACAATGAACTGCAGAAAGATAAGATGGCGGAGGCCTA
CAGTGAGATTGGGATGAAAGGCGAGCGCCGGAGGGGCAAGGGGCACGATGGCCTTTA
CCAGGGTCTCAGTACAGCCACCAAGGACACCTACGACGCCCTTCACATGCAGGCCCTG
CCCCCTCGCAGGAGAAAAAGAGGAAGCGGAGCTACTAACTTCAGCCTGCTGAAGCAG
GCTGGAGACGTGGAGGAGAACCCTGGACCTACGCGTGGAGGTGGCGCCACCATGGTG
AGCAAGGGCGAGGAGCTGTTCACCGGGGTGGTGCCCATCCTGGTCGAGCTGGACGGC
GACGTAAACGGCCACAAGTTCAGCGTGTCCGGCGAGGGCGAGGGCGATGCCACCTAC
GGCAAGCTGACCCTGAAGTTCATCTGCACCACCGGCAAGCTGCCCGTGCCCTGGCCC
ACCCTCGTGACCACCCTGACCTACGGCGTGCAGTGCTTCAGCCGCTACCCCGACCACA
TGAAGCAGCACGACTTCTTCAAGTCCGCCATGCCCGAAGGCTACGTCCAGGAGCGCAC
CATCTTCTTCAAGGACGACGGCAACTACAAGACCCGCGCCGAGGTGAAGTTCGAGGGC
GACACCCTGGTGAACCGCATCGAGCTGAAGGGCATCGACTTCAAGGAGGACGGCAAC
ATCCTGGGGCACAAGCTGGAGTACAACTACAACAGCCACAACGTCTATATCATGGCCG
ACAAGCAGAAGAACGGCATCAAGGTGAACTTCAAGATCCGCCACAACATCGAGGACGG
CAGCGTGCAGCTCGCCGACCACTACCAGCAGAACACCCCCATCGGCGACGGCCCCGT
GCTGCTGCCCGACAACCACTACCTGAGCACCCAGTCCAAGCTGAGCAAAGACCCCAAC
GAGAAGCGCGATCACATGGTCCTGCTGGAGTTCGTGACCGCCGCCGGGATCACTCTC
GGCATGGACGAGCTGTACAAGTAA
-Illustrative nucleotide sequence encoding illustrative CAR 4: CAR
containing CD8 leader, ASGR1 VH, CD8α hinge, CD28 transmembrane, CD28
cytoplasmic, CD3z cytoplasmic, FP2A domain, GFP
SEQ ID NO: 156
ATGGCCTTACCAGTGACCGCCTTGCTCCTGCCGCTGGCCTTGCTGCTCCACGCCGCCA
GGCCGGAGGTGCAGCTGTTGGAGTCTGGGGGAGGCTTGGTACAGCCTGGGGGGTCC
CTGCGTCTCTCCTGTGCAGCCTCCGGATTCACCTTTGAGAAGTATGCGATGGCGTGGG
TCCGCCAGGCCCCAGGGAAGGGTCTGGAGTGGGTCTCACGGATTTCGGCGAGGGGTG
TGACGACATACTACGCAGACTCCGTGAAGGGCCGGTTCACCATCTCCCGCGACAATTC
CAAGAACACGCTGTATCTGCAAATGAACAGCCTGCGTGCTGAGGACACCGCGGTATAT
TACTGTGCGAAACATAAGCGGCACGAGCATACTCGTTTTGACTCCTGGGGTCAGGGAA
CCCTGGTCACCGTCTCGAGCGCGGCCGCCATCGAGGTGGAGCAGAAGCTGATCAGCG
AGGAGGACCTGCTGGACAACGAGAAGAGCAACGGCACCATCATCCACGTGAAGGGCA
AGCACCTGTGCCCCAGCCCCCTGTTCCCCGGCCCCAGCAAGCCCTTCTGGGTGCTGG
TGGTGGTGGGCGGCGTGCTGGCCTGCTACAGCCTGCTGGTGACCGTGGCCTTCATCA
TCTTCTGGGTGCGGAGCAAGCGGAGCCGGCTGCTGCACAGCGACTACATGAACATGA
CCCCCCGGCGGCCTGGGCCCACCCGCAAGCATTACCAGCCCTATGCCCCACCACGCG
ACTTCGCAGCCTATCGCTCCAGAGTGAAGTTCAGCAGGAGCGCAGACGCCCCCGCGT
ACCAGCAGGGCCAGAACCAGCTCTATAACGAGCTCAATCTAGGACGAAGAGAGGAGTA
CGATGTTTTGGACAAGAGACGTGGCCGGGACCCTGAGATGGGGGGAAAGCCGAGAAG
GAAGAACCCTCAGGAAGGCCTGTACAATGAACTGCAGAAAGATAAGATGGCGGAGGCC
TACAGTGAGATTGGGATGAAAGGCGAGCGCCGGAGGGGCAAGGGGCACGATGGCCTT
TACCAGGGTCTCAGTACAGCCACCAAGGACACCTACGACGCCCTTCACATGCAGGCCC
TGCCCCCTCGCAGGAGAAAAAGAGGAAGCGGAGCTACTAACTTCAGCCTGCTGAAGCA
GGCTGGAGACGTGGAGGAGAACCCTGGACCTACGCGTGGAGGTGGCGCCACCATGGT
GAGCAAGGGCGAGGAGCTGTTCACCGGGGTGGTGCCCATCCTGGTCGAGCTGGACG
GCGACGTAAACGGCCACAAGTTCAGCGTGTCCGGCGAGGGCGAGGGCGATGCCACCT
ACGGCAAGCTGACCCTGAAGTTCATCTGCACCACCGGCAAGCTGCCCGTGCCCTGGC
CCACCCTCGTGACCACCCTGACCTACGGCGTGCAGTGCTTCAGCCGCTACCCCGACCA
CATGAAGCAGCACGACTTCTTCAAGTCCGCCATGCCCGAAGGCTACGTCCAGGAGCGC
ACCATCTTCTTCAAGGACGACGGCAACTACAAGACCCGCGCCGAGGTGAAGTTCGAGG
GCGACACCCTGGTGAACCGCATCGAGCTGAAGGGCATCGACTTCAAGGAGGACGGCA
ACATCCTGGGGCACAAGCTGGAGTACAACTACAACAGCCACAACGTCTATATCATGGC
CGACAAGCAGAAGAACGGCATCAAGGTGAACTTCAAGATCCGCCACAACATCGAGGAC
GGCAGCGTGCAGCTCGCCGACCACTACCAGCAGAACACCCCCATCGGCGACGGCCCC
GTGCTGCTGCCCGACAACCACTACCTGAGCACCCAGTCCAAGCTGAGCAAAGACCCCA
ACGAGAAGCGCGATCACATGGTCCTGCTGGAGTTCGTGACCGCCGCCGGGATCACTC
TCGGCATGGACGAGCTGTACAAGTAA
-Illustrative nucleotide sequence encoding illustrative CAR 5: CAR
containing CD8 leader, ASGR1 VH (VH1, SEQ ID NO: 74), linker, ASGR1 VL
(VK1, SEQ ID NO: 78), CD28 hinge with c-Myc tag, CD28 transmembrane,
CD28 cytoplasmic, CD3z cytoplasmic
SEQ ID NO: 170
ATGGCCTTACCAGTGACCGCCTTGCTCCTGCCGCTGGCCTTGCTGCTCCACGCCGCCAGGCCGGAGGT
GCAGCTGTTGGAGTCTGGGGGAGGCTTGGTACAGCCTGGGGGGTCCCTGCGTCTCTCCTGTGCAGCCT
CCGGATTCACCTTTGAGAAGTATGCGATGGCGTGGGTCCGCCAGGCCCCAGGGAAGGGTCTGGAGTGG
GTCTCACGGATTTCGGCGAGGGGTGTGACGACATACTACGCAGACTCCGTGAAGGGCCGGTTCACCAT
CTCCCGCGACAATTCCAAGAACACGCTGTATCTGCAAATGAACAGCCTGCGTGCTGAGGACACCGCGG
TATATTACTGTGCGAAACATAAGCGGCACGAGCATACTCGTTTTGACTCCTGGGGTCAGGGAACCCTG
GTCACCGTCTCGAGCCTGGTGACCGTGAGCAGCGGCGGCGGAGGCAGCGGTGGCGGAGGCAGCGGCGG
AGGCGGTAGCGACATCCAGATGACCCAGTCTCCATCCTCCCTGTCTGCATCTGTAGGAGACCGTGTCA
CCATTACTTGCCGGGCAAGTCAGGCGATTGGGCGGTGGTTATTGTGGTATCAGCAGAAACCAGGGAAA
GCCCCTAAGCTCCTGATCGGTCCGGGTTCCCGGTTGCGAAGTGGGGTCCCATCACGTTTCAGTGGCAG
TGGATCTGGGACAGATTTCACTCTCACCATCAGCAGTCTACAACCTGAAGATTTTGTTACGTACTACT
GTCAACAGGCGTATGCCTGGCCTCCGACGTTCGGCCAAGGGACCAAGGTGGAAATCAAACGGGCGGCC
GCCATCGAGGTGGAGCAGAAGCTGATCAGCGAGGAGGACCTGCTGGACAACGAGAAGAGCAACGGCAC
CATCATCCACGTGAAGGGCAAGCACCTGTGCCCCAGCCCCCTGTTCCCCGGCCCCAGCAAGCCCTTCT
GGGTGCTGGTGGTGGTGGGCGGCGTGCTGGCCTGCTACAGCCTGCTGGTGACCGTGGCCTTCATCATC
TTCTGGGTGCGGAGCAAGCGGAGCCGGCTGCTGCACAGCGACTACATGAACATGACCCCCCGGCGGCC
TGGGCCCACCCGCAAGCATTACCAGCCCTATGCCCCACCACGCGACTTCGCAGCCTATCGCTCCAGAG
TGAAGTTCAGCAGGAGCGCAGACGCCCCCGCGTACCAGCAGGGCCAGAACCAGCTCTATAACGAGCTC
AATCTAGGACGAAGAGAGGAGTACGATGTTTTGGACAAGAGACGTGGCCGGGACCCTGAGATGGGGGG
AAAGCCGAGAAGGAAGAACCCTCAGGAAGGCCTGTACAATGAACTGCAGAAAGATAAGATGGCGGAGG
CCTACAGTGAGATTGGGATGAAAGGCGAGCGCCGGAGGGGCAAGGGGCACGATGGCCTTTACCAGGGT
CTCAGTACAGCCACCAAGGACACCTACGACGCCCTTCACATGCAGGCCCTGCCCCCTCGC
-Illustrative nucleotide sequence encoding illustrative CAR 6: CAR
containing CD8 leader, ASGR1 VH (VH2, SEQ ID NO: 75), linker, ASGR1 VL
(VK2, SEQ ID NO: 79), CD28 hinge with c-Myc tag, CD28 transmembrane,
CD28 cytoplasmic, CD3z cytoplasmic
SEQ ID NO: 171
ATGGCCTTACCAGTGACCGCCTTGCTCCTGCCGCTGGCCTTGCTGCTCCACGCCGCCAGGCCGGAGTT
GCAGCTGTTGGAGTTTGGGGGAGGCTTGGTACAGCCTGGGGGGTCCCTGCGTCTCTCCTGTACAACCT
CCGGATTCACCTTTTCGAGGTATACTATGGGTTGGGTCCGCCAGGCTCCAGGGAAGGGTCTAGAGTGG
GTCTCAGCCATTGGCCCGCCCGGGTCGAACACATACTACGCAGACTCCGTGAAGGGTCGGTTCACCAT
CTCCCGCGACAATTCCAAGAACACGCTGTATCTGCAAATGAACAGCCTGCGTGCCGAGGACACCGCGG
TATATTACTGTGCGAAATGGGTGATGTTGCGGGGGCGCTTTGACTACTGGGGTCAGGGAACCCTGGTC
ACCGTCTCGAGCCTGGTGACCGTGAGCAGCGGCGGCGGAGGCAGCGGTGGCGGAGGCAGCGGCGGAGG
CGGTAGCGACATCCAGATGACCCAGTCCCCATCCTCCCTGTCTGCATCTGTAGGAGACCGTGTCACCA
TTACTTGCCGGGCAAGTCAGGCGATTGGGCGGTGGTTATTGTGGTATCAGCAGAAACCAGGGAAAGCC
CCTAAGCACCTGATCGGTCCGGGTTCCCGGTTGCAAAGTGGGGTCCCATCACGTTTCAGTGGCAGTGG
ATCTGGGACAGATTTCACTCTCACCATCAGCAGTCTGCAACCTGAAGATTTTGCTACGTACTACTGTC
AACAGGCGTATCAGCTGCCTGTCACGTTCGGCCAAGGGACCAAGGTGGAAATCAAACGGGCGGCCGCC
ATCGAGGTGGAGCAGAAGCTGATCAGCGAGGAGGACCTGCTGGACAACGAGAAGAGCAACGGCACCAT
CATCCACGTGAAGGGCAAGCACCTGTGCCCCAGCCCCCTGTTCCCCGGCCCCAGCAAGCCCTTCTGGG
TGCTGGTGGTGGTGGGCGGCGTGCTGGCCTGCTACAGCCTGCTGGTGACCGTGGCCTTCATCATCTTC
TGGGTGCGGAGCAAGCGGAGCCGGCTGCTGCACAGCGACTACATGAACATGACCCCCCGGCGGCCTGG
GCCCACCCGCAAGCATTACCAGCCCTATGCCCCACCACGCGACTTCGCAGCCTATCGCTCCAGAGTGA
AGTTCAGCAGGAGCGCAGACGCCCCCGCGTACCAGCAGGGCCAGAACCAGCTCTATAACGAGCTCAAT
CTAGGACGAAGAGAGGAGTACGATGTTTTGGACAAGAGACGTGGCCGGGACCCTGAGATGGGGGGAAA
GCCGAGAAGGAAGAACCCTCAGGAAGGCCTGTACAATGAACTGCAGAAAGATAAGATGGCGGAGGCCT
ACAGTGAGATTGGGATGAAAGGCGAGCGCCGGAGGGGCAAGGGGCACGATGGCCTTTACCAGGGTCTC
AGTACAGCCACCAAGGACACCTACGACGCCCTTCACATGCAGGCCCTGCCCCCTCGC
-Illustrative nucleotide sequence encoding illustrative CAR 7: CAR
containing CD8 leader, ASGR1 VH (VH3, SEQ ID NO: 76), linker, ASGR1 VL
(VK3, SEQ ID NO: 80), CD28 hinge with c-Myc tag, CD28 transmembrane,
CD28 cytoplasmic, CD3z cytoplasmic
SEQ ID NO: 172
ATGGCCTTACCAGTGACCGCCTTGCTCCTGCCGCTGGCCTTGCTGCTCCACGCCGCCAGGCCGGAGGT
GCAGCTGTTGGAGTCTGGGGGAGGCTTGGTACAGCCTGGGGGGTCCCTGCGTCTCTCCTGTGCAGCCT
CCGGATTCACCTTTGAGGATTATGGTATGGGGTGGGTCCGCCAGGCTCCAGGAAAGGGTCTAGAGTGG
GTCTCAGCGATTGGCCGCAACGGGTCGCAGACATACTACGCAGACTCCGTGAAGGGCCGGTTCACCAT
CTCCCGCGACAATTCCAAGAACACGCTGTATCTGCAAATGAACAGCCTGCGTGCCGAGGATACCGCGG
TATATTACTGTGCGAAACTTCGGAGGGGGCGGGGTCTGAATACGTTTACGTTAGACTACTGGGGTCAA
GGAACCCTGGTCACCGTCTCAAGCCTGGTGACCGTGAGCAGCGGCGGCGGAGGCAGCGGTGGCGGAGG
CAGCGGCGGAGGCGGTAGCGACATCCAGATGACCCAGTCCCCATCCTCCCTGTCTGCATCTGTAGGAG
ACCGTGTCACCATTACTTGCCGGGCAAGTCAGGCGATTGGGCGGTGGTTATTGTGGTATCAGCAGAAA
CCAGGGAAAGCCCCTAAGCTCCTGATCGGTCCGGGTTCCCGGTTGCAAAGTGGGGTCCCATCACGTTT
CAGTGGCAGTGGATCTGGGACAGATTTCACTCTCACCATCGGCAGTCTGCAACCTGAAGATTTTGCTA
CGTACTACTGTCAACAGGCGTATAGTCTGCCTCCGACGTTCGGCCAAGGGACCAAGGTGGAAATCAAA
CGGGCGGCCGCCATCGAGGTGGAGCAGAAGCTGATCAGCGAGGAGGACCTGCTGGACAACGAGAAGAG
CAACGGCACCATCATCCACGTGAAGGGCAAGCACCTGTGCCCCAGCCCCCTGTTCCCCGGCCCCAGCA
AGCCCTTCTGGGTGCTGGTGGTGGTGGGCGGCGTGCTGGCCTGCTACAGCCTGCTGGTGACCGTGGCC
TTCATCATCTTCTGGGTGCGGAGCAAGCGGAGCCGGCTGCTGCACAGCGACTACATGAACATGACCCC
CCGGCGGCCTGGGCCCACCCGCAAGCATTACCAGCCCTATGCCCCACCACGCGACTTCGCAGCCTATC
GCTCCAGAGTGAAGTTCAGCAGGAGCGCAGACGCCCCCGCGTACCAGCAGGGCCAGAACCAGCTCTAT
AACGAGCTCAATCTAGGACGAAGAGAGGAGTACGATGTTTTGGACAAGAGACGTGGCCGGGACCCTGA
GATGGGGGGAAAGCCGAGAAGGAAGAACCCTCAGGAAGGCCTGTACAATGAACTGCAGAAAGATAAGA
TGGCGGAGGCCTACAGTGAGATTGGGATGAAAGGCGAGCGCCGGAGGGGCAAGGGGCACGATGGCCTT
TACCAGGGTCTCAGTACAGCCACCAAGGACACCTACGACGCCCTTCACATGCAGGCCCTGCCCCCTCG
C

As discussed previously, polynucleotides of the invention may also encode for polypeptides in addition to the CAR as defined herein, e.g. a reporter or marker polypeptide or protein. Particularly, polynucleotides of the invention may additionally encode a FOXP3 polypeptide.

In one embodiment, a Treg of the invention may comprise one or more polynucleotides encoding a CAR of the invention and a FOXP3 polypeptide (an exogenous FOXP3 polypeptide), and thus a Treg of the invention may be produced by introducing into the cell a polynucleotide encoding a CAR of the invention and a FOXP3 polypeptide. It will be appreciated that the CAR of the invention and the FOXP3 polypeptide may be encoded by a single polynucleotide sequence or by different polynucleotides.

“FOXP3” is the abbreviated name of the forkhead box P3 protein. FOXP3 is a member of the FOX protein family of transcription factors and functions as a master regulator of the regulatory pathway in the development and function of regulatory T cells. “FOXP3” as used herein encompasses variants, isoforms, and functional fragments of FOXP3.

A “FOXP3 polypeptide” is a polypeptide having FOXP3 activity i.e. a polypeptide able to bind FOXP3 target DNA and function as a transcription factor regulating development and function of Tregs. Particularly, a FOXP3 polypeptide may have the same or similar activity to wildtype FOXP3 (SEQ ID NO. 157), e.g. may have at least 40, 50, 60, 70, 80, 90, 95, 100, 110, 120, 130, 140 or 150% of the activity of the wildtype FOXP3 polypeptide. Thus, a FOXP3 polypeptide encoded by a nucleotide sequence described herein may have increased or decreased activity compared to wildtype FOXP3. Techniques for measuring transcription factor activity are well known in the art. For example, transcription factor DNA-binding activity may be measured by ChIP. The transcription regulatory activity of a transcription factor may be measured by quantifying the level of expression of genes which it regulates. Gene expression may be quantified by measuring the levels of mRNA and/or protein produced from the gene using techniques such as Northern blotting, SAGE, qPCR, HPLC, LC/MS, Western blotting or ELISA. Genes regulated by FOXP3 include cytokines such as IL-2, IL-4 and IFN-γ (Siegler et al. Annu. Rev. Immunol. 2006, 24: 209-26, incorporated herein by reference). As discussed in detail below and previously, FOXP3 or a FOXP3 polypeptide includes functional fragments, variants, and isoforms thereof, e.g. of SEQ ID NO. 157.

A “functional fragment of FOXP3” may refer to a portion or region of a FOXP3 polypeptide or a polynucleotide (i.e. nucleotide sequence) encoding a FOXP3 polypeptide that has the same or similar activity to the full-length FOXP3 polypeptide or polynucleotide. The functional fragment may have at least 40%, at least 50%, at least 60%, at least 70%, at least 80%, at least 90%, at least 95%, or 100% of the activity of the full-length FOXP3 polypeptide or polynucleotide. A person skilled in the art would be able to generate functional fragments based on the known structural and functional features of FOXP3. These are described, for instance, in Song, X., et al., 2012. Cell reports, 1(6), pp. 665-675; Lopes, J. E., et al., 2006. The Journal of Immunology, 177(5), pp. 3133-3142; and Lozano, T., et al, 2013. Frontiers in oncology, 3, p. 294. Further, a N and C terminally truncated FOXP3 fragment is described within WO2019/241549 (incorporated herein by reference), for example, having the sequence SEQ ID NO. 157 as discussed below.

A “FOXP3 variant” may include an amino acid sequence or a nucleotide sequence which may be at least 50%, at least 55%, at least 65%, at least 70%, at least 75%, at least 80%, at least 85% or at least 90% identical, preferably at least 95% or at least 97% or at least 99% identical to a FOXP3 polypeptide or a polynucleotide encoding a FOXP3 polypeptide, e.g. to SEQ ID NO. 157. FOXP3 variants may have the same or similar activity to a wildtype FOXP3 polypeptide or polynucleotide, e.g. may have at least 40, 50, 60, 70, 80, 90, 95, 100, 110, 120, 130, 140 or 150% of the activity of a wildtype FOXP3 polypeptide or polynucleotide. A person skilled in the art would be able to generate FOXP3 variants based on the known structural and functional features of FOXP3 and/or using conservative substitutions. FOXP3 variants may have similar or the same turnover time (or degradation rate) within a Treg cell as compared to wildtype FOXP3, e.g. at least 40, 50, 60, 70, 80, 90, 95, 99 or 100% of the turnover time (or degradation rate) of wildtype FOXP3 in a Treg.

Some FOXP3 variants may have a reduced turnover time (or degradation rate) as compared to wildtype FOXP3, for example, FOXP3 variants having amino acid substitutions at amino acid 418 and/or 422 of SEQ ID NO. 157, for example S418E and/or S422A, as described in WO2019/241549 (incorporated herein by reference) and are set out in SEQ ID NOs 158 to 160, which represent the aa418, aa422 and aa418 and aa422 mutants respectively.

Suitably, the FOXP3 polypeptide encoded by a nucleic acid molecule, construct or vector as described herein may comprise or consist of the polypeptide sequence of a human FOXP3, such as UniProtKB accession Q9BZS1 (SEQ ID NO: 157), or a functional fragment or variant thereof.

In some embodiments of the invention, the FOXP3 polypeptide comprises or consists of an amino acid sequence which is at least 70% identical to SEQ ID NO: 157 or a functional fragment thereof. Suitably, the FOXP3 polypeptide comprises or consists of an amino acid sequence which is at least 80%, at least 85%, at least 90%, at least 95%, at least 98% or at least 99% identical to SEQ ID NO: 157 or a functional fragment thereof. In some embodiments, the FOXP3 polypeptide comprises or consists of SEQ ID NO: 157 or a functional fragment thereof.

In some embodiments, as discussed above, the FOXP3 polypeptide may comprise mutations at residues 418 and/or 422 of SEQ ID NO. 157, as set out in SEQ ID NO. 158, SEQ ID NO. 159, or SEQ ID NO. 160.

In some embodiments of the invention, the FOXP3 polypeptide may be truncated at the N and/or C terminal ends, resulting in the production of a functional fragment. Particularly, an N and C terminally truncated functional fragment of FOXP3 may comprise or consist of an amino acid sequence of SEQ ID NO. 161 or a functional variant thereof having at least 80, 85, 90, 95 or 99% identity thereto.

Suitably, the FOXP3 polypeptide may be a variant of SEQ ID NO: 157, for example a natural variant. Suitably, the FOXP3 polypeptide is an isoform of SEQ ID NO: 157. For example, the FOXP3 polypeptide may comprise a deletion of amino acid positions 72-106 relative to SEQ ID NO: 157. Alternatively, the FOXP3 polypeptide may comprise a deletion of amino acid positions 246-272 relative to SEQ ID NO: 157.

Suitably, the FOXP3 polypeptide comprises SEQ ID NO: 162 or a functional fragment thereof. SEQ ID NO: 162 represents an Illustrative FOXP3 polypeptide.

Suitably the FOXP3 polypeptide comprises or consists of an amino acid sequence which is at least 70% identical to SEQ ID NO: 162 or a functional fragment thereof. Suitably, the FOXP3 polypeptide comprises an amino acid sequence which is at least 80%, at least 85%, at least 90%, at least 95%, at least 98% or at least 99% identical to SEQ ID NO: 162 or a functional fragment thereof. In some embodiments, the FOXP3 polypeptide comprises or consists of SEQ ID NO: 162 or a functional fragment thereof.

Suitably, the FOXP3 polypeptide may be a variant of SEQ ID NO: 162, for example a natural variant. Suitably, the FOXP3 polypeptide is an isoform of SEQ ID NO: 162 or a functional fragment thereof. For example, the FOXP3 polypeptide may comprise a deletion of amino acid positions 72-106 relative to SEQ ID NO: 162. Alternatively, the FOXP3 polypeptide may comprise a deletion of amino acid positions 246-272 relative to SEQ ID NO: 162.

Suitably, the polynucleotide encoding a FOXP3 polypeptide comprises or consists of a nucleotide sequence set forth in SEQ ID NO: 163, which represents an illustrative FOXP3 nucleotide sequence.

In some embodiments of the invention, the polynucleotide encoding the FOXP3 polypeptide or variant comprises nucleotide sequence which is at least 70% identical to SEQ ID NO: 163 or a fragment thereof which encodes a functional FOXP3 polypeptide. Suitably, the polynucleotide encoding the FOXP3 polypeptide or variant comprises a polynucleotide sequence which is at least 80%, at least 85%, at least 90%, at least 95%, at least 98% or at least 99% identical to SEQ ID NO: 163 or a fragment thereof which encodes a functional FOXP3 polypeptide. In some embodiments of the invention, the polynucleotide encoding the FOXP3 polypeptide or variant comprises or consists of SEQ ID NO: 163 or a fragment thereof which encodes a functional FOXP3 polypeptide.

Suitably, the polynucleotide encoding a FOXP3 polypeptide comprises or consists of a polynucleotide sequence set forth in SEQ ID NO: 164, which represents another illustrative FOXP3 nucleotide.

In some embodiments of the invention, the polynucleotide encoding the FOXP3 polypeptide or variant comprises a nucleotide sequence which is at least 70% identical to SEQ ID NO: 164 or a fragment thereof which encodes a functional FOXP3 polypeptide. Suitably, the polynucleotide encoding the FOXP3 polypeptide or variant comprises a polynucleotide sequence which is at least 80%, at least 85%, at least 90%, at least 95%, at least 98% or at least 99% identical to SEQ ID NO: 164 or a fragment thereof which encodes a functional FOXP3 polypeptide. In some embodiments of the invention, the polynucleotide encoding the FOXP3 polypeptide or variant comprises or consists of SEQ ID NO: 164 or a fragment thereof which encodes a functional FOXP3 polypeptide.

Suitably, the polynucleotide encoding the FOXP3 polypeptide or functional fragment or variant thereof may be codon optimised. Suitably, the polynucleotide encoding the FOXP3 polypeptide or functional fragment or variant thereof may be codon optimised for expression in a human cell.

SEQ ID NO. 157 FOXP3, UniProtKB accession Q9BZS1:
MPNPRPGKPSAPSLALGPSPGASPSWRAAPKASDLLGARGPGGTFQGRDLRGGAHASSS
SLNPMPPSQLQLPTLPLVMVAPSGARLGPLPHLQALLQDRPHFMHQLSTVDAHARTPVLQ
VHPLESPAMISLTPPTTATGVFSLKARPGLPPGINVASLEWVSREPALLCTFPNPSAPRKDS
TLSAVPQSSYPLLANGVCKWPGCEKVFEEPEDFLKHCQADHLLDEKGRAQCLLQREMVQS
LEQQLVLEKEKLSAMQAHLAGKMALTKASSVASSDKGSCCIVAAGSQGPVVPAWSGPREA
PDSLFAVRRHLWGSHGNSTFPEFLHNMDYFKFHNMRPPFTYATLIRWAILEAPEKQRTLNEI
YHWFTRMFAFFRNHPATWKNAIRHNLSLHKCFVRVESEKGAVWTVDELEFRKKRSQRPSR
CSNPTPGP
SEQ ID NO. 158 FOXP3, aa418 mutant:
MPNPRPGKPSAPSLALGPSPGASPSWRAAPKASDLLGARGPGGTFQGRDLRGGAHASSS
SLNPMPPSQLQLPTLPLVMVAPSGARLGPLPHLQALLQDRPHFMHQLSTVDAHARTPVLQ
VHPLESPAMISLTPPTTATGVFSLKARPGLPPGINVASLEWVSREPALLCTFPNPSAPRKDS
TLSAVPQSSYPLLANGVCKWPGCEKVFEEPEDFLKHCQADHLLDEKGRAQCLLQREMVQS
LEQQLVLEKEKLSAMQAHLAGKMALTKASSVASSDKGSCCIVAAGSQGPVVPAWSGPREA
PDSLFAVRRHLWGSHGNSTFPEFLHNMDYFKFHNMRPPFTYATLIRWAILEAPEKQRTLNEI
YHWFTRMFAFFRNHPATWKNAIRHNLSLHKCFVRVESEKGAVWTVDELEFRKKREQRPSR
CSNPTPGP
SEQ ID NO. 159 FOXP3, aa422 mutant:
MPNPRPGKPSAPSLALGPSPGASPSWRAAPKASDLLGARGPGGTFQGRDLRGGAHASSS
SLNPMPPSQLQLPTLPLVMVAPSGARLGPLPHLQALLQDRPHFMHQLSTVDAHARTPVLQ
VHPLESPAMISLTPPTTATGVFSLKARPGLPPGINVASLEWVSREPALLCTFPNPSAPRKDS
TLSAVPQSSYPLLANGVCKWPGCEKVFEEPEDFLKHCQADHLLDEKGRAQCLLQREMVQS
LEQQLVLEKEKLSAMQAHLAGKMALTKASSVASSDKGSCCIVAAGSQGPVVPAWSGPREA
PDSLFAVRRHLWGSHGNSTFPEFLHNMDYFKFHNMRPPFTYATLIRWAILEAPEKQRTLNEI
YHWFTRMFAFFRNHPATWKNAIRHNLSLHKCFVRVESEKGAVWTVDELEFRKKRSQRPAR
CSNPTPGP
SEQ ID NO. 160 FOXP3, aa 418 and 422 mutant:
MPNPRPGKPSAPSLALGPSPGASPSWRAAPKASDLLGARGPGGTFQGRDLRGGAHASSS
SLNPMPPSQLQLPTLPLVMVAPSGARLGPLPHLQALLQDRPHFMHQLSTVDAHARTPVLQ
VHPLESPAMISLTPPTTATGVFSLKARPGLPPGINVASLEWVSREPALLCTFPNPSAPRKDS
TLSAVPQSSYPLLANGVCKWPGCEKVFEEPEDFLKHCQADHLLDEKGRAQCLLQREMVQS
LEQQLVLEKEKLSAMQAHLAGKMALTKASSVASSDKGSCCIVAAGSQGPVVPAWSGPREA
PDSLFAVRRHLWGSHGNSTFPEFLHNMDYFKFHNMRPPFTYATLIRWAILEAPEKQRTLNEI
YHWFTRMFAFFRNHPATWKNAIRHNLSLHKCFVRVESEKGAVWTVDELEFRKKREQRPAR
CSNPTPGP
SEQ ID NO. 161 FOXP3, truncated variant:
GGAHASSSSL NPMPPSQLQL PTLPLVMVAP SGARLGPLPH LQALLQDRPH
FMHQLSTVDA HARTPVLQVH PLESPAMISL TPPTTATGVF SLKARPGLPP GINVASLEWV
SREPALLCTF PNPSAPRKDS TLSAVPQSSY PLLANGVCKW PGCEKVFEEP
EDFLKHCQAD HLLDEKGRAQ CLLQREMVQS LEQQLVLEKE KLSAMQAHLA
GKMALTKASS VASSDKGSCC IVAAGSQGPV VPAWSGPREA PDSLFAVRRH
LWGSHGNSTF PEFLHNMDYF KFHNMRPPFT YATLIRWAIL EAPEKQRTLN
EIYHWFTRMF AFFRNHPATW KNAIRHNLSL HKCFVRVESE KGAVWTVDEL EF
SEQ ID NO. 162 FOXP3, illustrative variant:
MPNPRPGKPSAPSLALGPSPGASPSWRAAPKASDLLGARGPGGTFQGRDLRGGAHASSS
SLNPMPPSQLQLPTLPLVMVAPSGARLGPLPHLQALLQDRPHFMHQLSTVDAHARTPVLQ
VHPLESPAMISLTPPTTATGVFSLKARPGLPPGINVASLEWVSREPALLCTFPNPSAPRKDS
TLSAVPQSSYPLLANGVCKWPGCEKVFEEPEDFLKHCQADHLLDEKGRAQCLLQREMVQS
LEQVEELSAMQAHLAGKMALTKASSVASSDKGSCCIVAAGSQGPVVPAWSGPREAPDSLF
AVRRHLWGSHGNSTFPEFLHNMDYFKFHNMRPPFTYATLIRWAILEAPEKQRTLNEIYHWF
TRMFAFFRNHPATWKNAIRHNLSLHKCFVRVESEKGAVWTVDELEFRKKRSQRPSRCSNP
TPGPEGRGSLLTCGDVEEN
SEQ ID NO. 163 FOXP3, Illustrative FOXP3 polynucleotide:
ATGCCCAACCCCAGGCCTGGCAAGCCCTCGGCCCCTTCCTTGGCCCTTGGCCCATCC
CCAGGAGCCTCGCCCAGCTGGAGGGCTGCACCCAAAGCCTCAGACCTGCTGGGGGCC
CGGGGCCCAGGGGGAACCTTCCAGGGCCGAGATCTTCGAGGCGGGGCCCATGCCTC
CTCTTCTTCCTTGAACCCCATGCCACCATCGCAGCTGCAGCTGCCCACACTGCCCCTA
GTCATGGTGGCACCCTCCGGGGCACGGCTGGGCCCCTTGCCCCACTTACAGGCACTC
CTCCAGGACAGGCCACATTTCATGCACCAGCTCTCAACGGTGGATGCCCACGCCCGGA
CCCCTGTGCTGCAGGTGCACCCCCTGGAGAGCCCAGCCATGATCAGCCTCACACCAC
CCACCACCGCCACTGGGGTCTTCTCCCTCAAGGCCCGGCCTGGCCTCCCACCTGGGA
TCAACGTGGCCAGCCTGGAATGGGTGTCCAGGGAGCCGGCACTGCTCTGCACCTTCC
CAAATCCCAGTGCACCCAGGAAGGACAGCACCCTTTCGGCTGTGCCCCAGAGCTCCTA
CCCACTGCTGGCAAATGGTGTCTGCAAGTGGCCCGGATGTGAGAAGGTCTTCGAAGAG
CCAGAGGACTTCCTCAAGCACTGCCAGGCGGACCATCTTCTGGATGAGAAGGGCAGG
GCACAATGTCTCCTCCAGAGAGAGATGGTACAGTCTCTGGAGCAGCAGCTGGTGCTGG
AGAAGGAGAAGCTGAGTGCCATGCAGGCCCACCTGGCTGGGAAAATGGCACTGACCA
AGGCTTCATCTGTGGCATCATCCGACAAGGGCTCCTGCTGCATCGTAGCTGCTGGCAG
CCAAGGCCCTGTCGTCCCAGCCTGGTCTGGCCCCCGGGAGGCCCCTGACAGCCTGTT
TGCTGTCCGGAGGCACCTGTGGGGTAGCCATGGAAACAGCACATTCCCAGAGTTCCTC
CACAACATGGACTACTTCAAGTTCCACAACATGCGACCCCCTTTCACCTACGCCACGCT
CATCCGCTGGGCCATCCTGGAGGCTCCAGAGAAGCAGCGGACACTCAATGAGATCTAC
CACTGGTTCACACGCATGTTTGCCTTCTTCAGAAACCATCCTGCCACCTGGAAGAACGC
CATCCGCCACAACCTGAGTCTGCACAAGTGCTTTGTGCGGGTGGAGAGCGAGAAGGG
GGCTGTGTGGACCGTGGATGAGCTGGAGTTCCGCAAGAAACGGAGCCAGAGGCCCAG
CAGGTGTTCCAACCCTACACCTGGCCCCTGA
SEQ ID NO. 164 FOXP3, Illustrative FOXP3 polynucleotide:
GAATTCGTCGACATGCCCAACCCCAGACCCGGCAAGCCTTCTGCCCCTTCTCTGGCCC
TGGGACCATCTCCTGGCGCCTCCCCATCTTGGAGAGCCGCCCCTAAAGCCAGCGATCT
GCTGGGAGCTAGAGGCCCTGGCGGCACATTCCAGGGCAGAGATCTGAGAGGCGGAG
CCCACGCCTCTAGCAGCAGCCTGAATCCCATGCCCCCTAGCCAGCTGCAGCTGCCTAC
ACTGCCTCTCGTGATGGTGGCCCCTAGCGGAGCTAGACTGGGCCCTCTGCCTCATCTG
CAGGCTCTGCTGCAGGACCGGCCCCACTTTATGCACCAGCTGAGCACCGTGGACGCC
CACGCCAGAACACCTGTGCTGCAGGTGCACCCCCTGGAAAGCCCTGCCATGATCAGC
CTGACCCCTCCAACCACAGCCACCGGCGTGTTCAGCCTGAAGGCCAGACCTGGACTG
CCCCCTGGCATCAATGTGGCCAGCCTGGAATGGGTGTCCCGCGAACCTGCCCTGCTG
TGCACCTTCCCCAATCCTAGCGCCCCCAGAAAGGACAGCACACTGTCTGCCGTGCCCC
AGAGCAGCTATCCCCTGCTGGCTAACGGCGTGTGCAAGTGGCCTGGCTGCGAGAAGG
TGTTCGAGGAACCCGAGGACTTCCTGAAGCACTGCCAGGCCGACCATCTGCTGGACGA
GAAAGGCAGAGCCCAGTGCCTGCTGCAGCGCGAGATGGTGCAGTCCCTGGAACAGCA
GCTGGTGCTGGAAAAAGAAAAGCTGAGCGCCATGCAGGCCCACCTGGCCGGAAAGAT
GGCCCTGACAAAAGCCAGCAGCGTGGCCAGCTCCGACAAGGGCAGCTGTTGTATCGT
GGCCGCTGGCAGCCAGGGACCTGTGGTGCCTGCTTGGAGCGGACCTAGAGAGGCCC
CCGATAGCCTGTTTGCCGTGCGGAGACACCTGTGGGGCAGCCACGGCAACTCTACCTT
CCCCGAGTTCCTGCACAACATGGACTACTTCAAGTTCCACAACATGAGGCCCCCCTTCA
CCTACGCCACCCTGATCAGATGGGCCATTCTGGAAGCCCCCGAGAAGCAGCGGACCC
TGAACGAGATCTACCACTGGTTTACCCGGATGTTCGCCTTCTTCCGGAACCACCCCGC
CACCTGGAAGAACGCCATCCGGCACAATCTGAGCCTGCACAAGTGCTTCGTGCGGGTG
GAAAGCGAGAAGGGCGCCGTGTGGACAGTGGACGAGCTGGAATTTCGGAAGAAGCGG
TCCCAGAGGCCCAGCCGGTGTAGCAATCCTACACCTGGCCCTGAGGGCAGAGGAAGT
CTGCTAACATGCGGTGACGTCGAGGAGAATCC

Vectors

The present invention provides a vector encoding the CAR of the invention. The vector may comprise a polynucleotide of the invention, e.g. encoding a CAR of the invention and optionally encoding a further polypeptide, e.g. a FOXP3 polypeptide.

A vector is a tool that allows or facilitates the transfer of an entity from one environment to another. In accordance with the present invention, and by way of example, some vectors used in recombinant nucleic acid techniques allow entities, such as a segment of nucleic acid (e.g. a heterologous DNA segment, such as a heterologous cDNA segment), to be transferred into a target cell. Vectors may be non-viral or viral. Examples of vectors used in recombinant nucleic acid techniques include, but are not limited to, plasmids, mRNA molecules (e.g. in vitro transcribed mRNAs), chromosomes, artificial chromosomes and viruses. The vector may also be, for example, a naked nucleic acid (e.g. DNA). In its simplest form, the vector may itself be a nucleotide of interest.

The vectors used in the invention may be, for example, plasmid, mRNA or virus vectors and may include a promoter for the expression of a polynucleotide and optionally a regulator of the promoter.

Vectors comprising polynucleotides of the invention may be introduced into cells using a variety of techniques known in the art, such as transformation and transduction. Several techniques are known in the art, for example infection with recombinant viral vectors, such as retroviral, lentiviral, adenoviral, adeno-associated viral, baculoviral and herpes simplex viral vectors; direct injection of nucleic acids and biolistic transformation.

Non-viral delivery systems include but are not limited to DNA transfection methods. Here, transfection includes a process using a non-viral vector to deliver a gene to a target cell.

Non-viral delivery systems can include liposomal or amphipathic cell penetrating peptides, preferably complexed with a polynucleotide of the invention.

Typical transfection methods include electroporation, DNA biolistics, lipid-mediated transfection, compacted DNA-mediated transfection, liposomes, immunoliposomes, lipofectin, cationic agent-mediated transfection, cationic facial amphiphiles (CFAs) (Nat. Biotechnol. (1996) 14: 556) and combinations thereof.

Multiple vectors, e.g. encoding different CARs of the invention, or encoding a CAR of the invention and a further polypeptide could be used for transduction/transfection.

Method of Making a Cell

Engineered Tregs of the present invention may be generated by introducing DNA or RNA coding for the CAR as defined herein, by one of many means including transduction with a viral vector, or transfection with DNA or RNA.

The engineered Treg of the invention may be made by introducing to a Treg (e.g. by transduction or transfection) the polynucleotide or vector as defined herein. Alternatively, an engineered Treg of the invention may be made by introducing a polynucleotide or vector as defined herein to a cell which is not a Treg and converting (e.g. differentiating or reprogramming said cell) to have a Treg phenotype, as described further below. The polynucleotide or vector may be introduced prior to or after any conversion.

Suitably, the Treg may be from a sample isolated from a subject. The Treg may be further separated from the sample by any suitable method, for example magnetic separation.

The engineered Treg of the present invention may be generated by a method comprising the following steps:
(i) isolation of a cell-containing sample from a subject or provision of a cell-containing sample; and
(ii) transduction or transfection of the cell-containing sample with a polynucleotide, a nucleic acid, or a vector encoding the CAR of the invention, to provide a population of engineered cells.

Suitably, a Treg-enriched sample may be isolated from, enriched, and/or generated from the cell-containing sample prior to and/or after step (ii) of the method. For example, isolation, enrichment and/or generation of Tregs may be performed prior to and/or after step (ii) to isolate, enrich or generate a Treg-enriched sample. Isolation and/or enrichment may be performed after step (ii) to enrich for cells and/or Tregs comprising the CAR, the polynucleotide, and/or the vector of the present invention.

A Treg-enriched sample may be isolated or enriched by any method known to those of skill in the art, for example by FACS and/or magnetic bead sorting. A Treg-enriched sample may be generated from the cell-containing sample by any method known to those of skill in the art, for example from Tcon cells by introducing DNA or RNA coding for FOXP3 and/or from ex-vivo differentiation of inducible progenitor cells or embryonic progenitor cells.

Suitably, the cell is a Treg as defined herein.

Suitably, the engineered Treg of the present invention may be generated by a method comprising the following steps:
(i) isolation of a Treg-enriched sample from a subject or provision of a Treg-enriched sample; and
(ii) transduction or transfection of the Treg-enriched sample with a polynucleotide, a nucleic acid, or a vector encoding the CAR of the invention, to provide a population of engineered Treg cells according to the present invention.

The cells and/or Tregs may be activated and/or expanded prior to, or after, the introduction of a polynucleotide encoding the CAR as described herein, for example by treatment with an anti-CD3 monoclonal antibody or both anti-CD3 and anti-CD28 monoclonal antibodies.

The cells and/or Tregs may also be expanded in the presence of anti-CD3 and anti-CD28 monoclonal antibodies in combination with IL-2. Suitably, IL-2 may be substituted with IL-15. Other components which may be used in a Treg expansion protocol include, but are not limited to rapamycin, all-trans retinoic acid (ATRA) and TGFβ.

As used herein “activated” means that a cell or population of cells has been stimulated, causing the cell(s) to proliferate. As used herein “expanded” means that a cell or population of cells has been induced to proliferate. The expansion of a population of cells may be measured for example by counting the number of cells present in a population. The phenotype of the cells may be determined by methods known in the art such as flow cytometry.

The cells and/or Tregs may be washed after each step of the method, in particular after expansion.

The population of engineered cells or Tregs may be further enriched by any method known to those of skill in the art, for example by FACS and/or magnetic bead sorting.

The steps of the method of production may be performed in a closed and sterile cell culture system.

### EXAMPLES

The invention will now be further described by way of Examples, which are meant to serve to assist one of ordinary skill in the art in carrying out the invention and are not intended in any way to limit the scope of the invention.

### Example 1

### Design of Anti-ASGR1 CAR Constructs

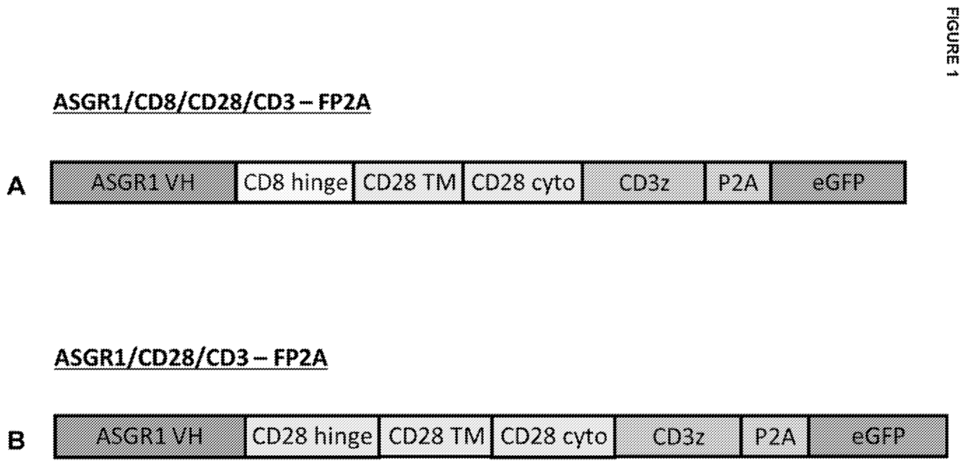

Chimeric antigen receptors (CAR) were designed comprising: an antigen recognition domain derived from a single-domain antibody (sdAb) known to specifically bind to ASGR; a transmembrane domain (TM) derived from CD28 (aa 153 to 179); and an intracellular signalling domain comprising the signalling domains of CD3ζ and CD28. Exemplary constructs are shown below and in FIG. 1.

Anti-ASGR1 CAR construct 1 comprising: CD8 leader, ASGR1 VH antigen recognition
domain, CD8α hinge domain, CD28 transmembrane, CD28 cytoplasmic signalling domain,
CD3z cytoplasmic signalling domain, FP2A domain, GFP
(SEQ ID NO: 151)
MALPVTALLLPLALLLHAARPEVQLLESGGGLVQPGGSLRLSCAASGFTFEKYAMAWRQA
PGKGLEWSRISARGVTTYYADSVKGRFTISRDNSKNTLYLQMNSLRAEDTAVYYCAKHKR
HEHTRFDSWGQGTLVTVSSTTTPAPRPPTPAPTIASQPLSLRPEACRPAAGGAVHTRGLDF
ACDFWVLVWGGVLACYSLLVTVAFIIFWVRSKRSRLLHSDYMNMTPRRPGPTRKHYQPYA
PPRDFAAYRSRVKFSRSADAPAYQQGQNQLYNELNLGRREEYDVLDKRRGRDPEMGGKP
RRKNPQEGLYNELQKDKMAEAYSEIGMKGERRRGKGHDGLYQGLSTATKDTYDALHMQA
LPPRRRKRGSGATNFSLLKQAGDVEENPGPTRGGGATMVSKGEELFTGVVPILVELDGDV
NGHKFSVSGEGEGDATYGKLTLKFICTTGKLPVPWPTLVTTLTYGVQCFSRYPDHMKQHD
FFKSAMPEGYVQERTIFFKDDGNYKTRAEVKFEGDTLVNRIELKGIDFKEDGNILGHKLEYN
YNSHNVYIMADKQKNGIKVNFKIRHNIEDGSVQLADHYQQNTPIGDGPVLLPDNHYLSTQSK
LSKDPNEKRDHMVLLEFVTAAGITLGMDELYK
Anti-ASGR1 CAR construct 2 comprising: CD8 leader, ASGR1 VH antigen recognition
domain, CD28 hinge domain with c-Myc tag, CD28 transmembrane, CD28 cytoplasmic
signalling domain, CD3z cytoplasmic signalling domain, FP2A domain, GFP
(SEQ ID NO: 152)
MALPVTALLLPLALLLHAARPEVQLLESGGGLVQPGGSLRLSCAASGFTFEKYAMAWVRQA
PGKGLEWSRISARGVTTYYADSVKGRFTISRDNSKNTLYLQMNSLRAEDTAVYYCAKHKR
HEHTRFDSWGQGTLVTVSSAAAIEVEQKLISEEDLLDNEKSNGTIIHVKGKHLCPSPLFPGP
SKPFWVLVVVGGVLACYSLLVTVAFIIFWRSKRSRLLHSDYMNMTPRRPGPTRKHYQPYA
PPRDFAAYRSRVKFSRSADAPAYQQGQNQLYNELNLGRREEYDVLDKRRGRDPEMGGKP
RRKNPQEGLYNELQKDKMAEAYSEIGMKGERRRGKGHDGLYQGLSTATKDTYDALHMQA
LPPRRRKRGSGATNFSLLKQAGDVEENPGPTRGGGATMVSKGEELFTGVVPILVELDGDV
NGHKFSVSGEGEGDATYGKLTLKFICTTGKLPVPWPTLVTTLTYGVQCFSRYPDHMKQHD
FFKSAMPEGYVQERTIFFKDDGNYKTRAEVKFEGDTLVNRIELKGIDFKEDGNILGHKLEYN
YNSHNVYIMADKQKNGIKVNFKIRHNIEDGSVQLADHYQQNTPIGDGPVLLPDNHYLSTQSK
LSKDPNEKRDHMVLLEFVTAAGITLGMDELYK

### Example 2

### Generation of Anti-ASGR1 CAR-Tregs

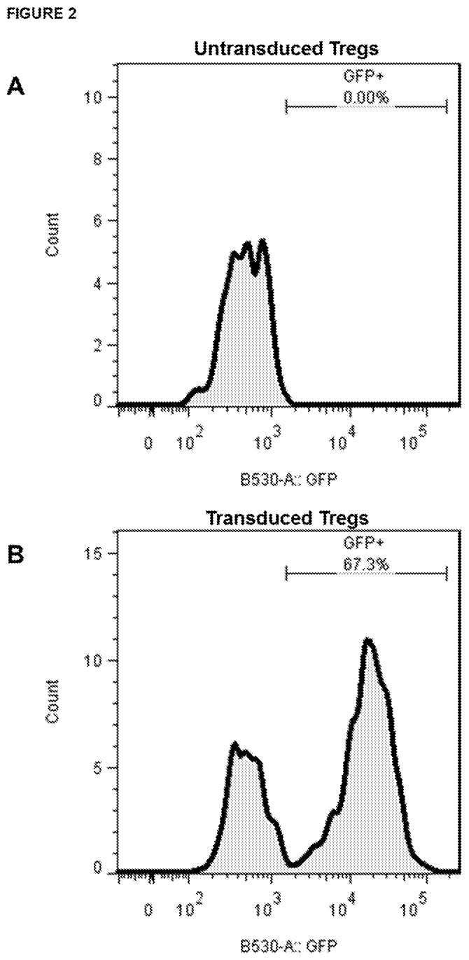

Anti-ASGR1 CAR-Tregs were generated. Isolated CD4+CD25hiCD127− were isolated and activated with anti-CD3/CD28 beads. Two days after activation Tregs were transduced with lentivirus containing the anti-ASGR1 CAR and the GFP reporter gene. Transduced and untransduced Tregs were cultured during 10 days and GFP was measured to assess transduction efficacy. FIG. 2 shows that 67.3% of transduced Tregs expressed GFP (i.e. contained the anti-ASGR1 CAR construct).

### Example 3

### Validation of ASGR1 Expression on HepG2 Cell Line

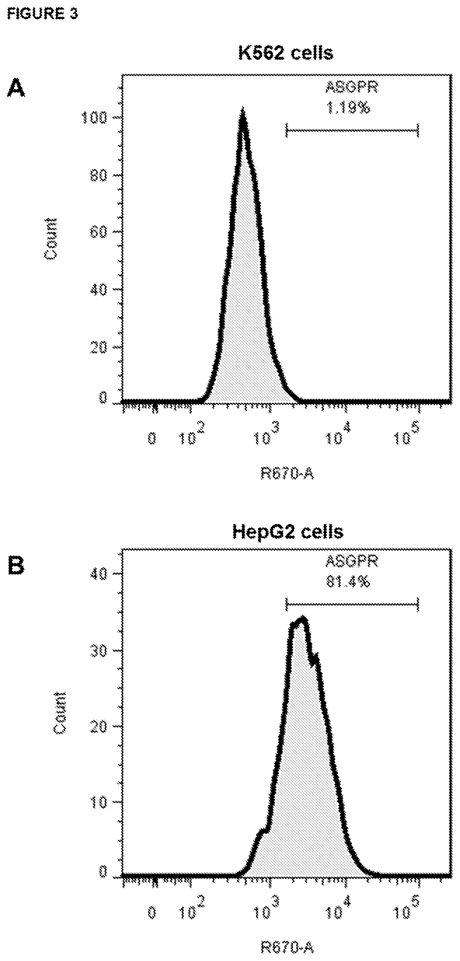

HepG2 cells are a suitable in vitro model system for the study of polarized human hepatocytes. Expression of ASGR1 in HepG2 was confirmed by FACS and compared to K562 cells, an immortalised human myelogenous leukaemia cell line. FIG. 3 shows that there was high expression of ASGR1 in HepG2 cells. HepG2 cells will be used as CAR-Treg target cell in following experiments

### Example 4

### Evaluation of the Antigen-Specificity of Anti-ASGR1 CAR-Tregs

We assessed the activation and proliferation of Tregs after culture with the cell line HepG2, which expresses ASGR1, and confirmed that only Tregs expressing the anti-ASGR1 CAR upregulate CD69 in response to ASGR1 stimulation.

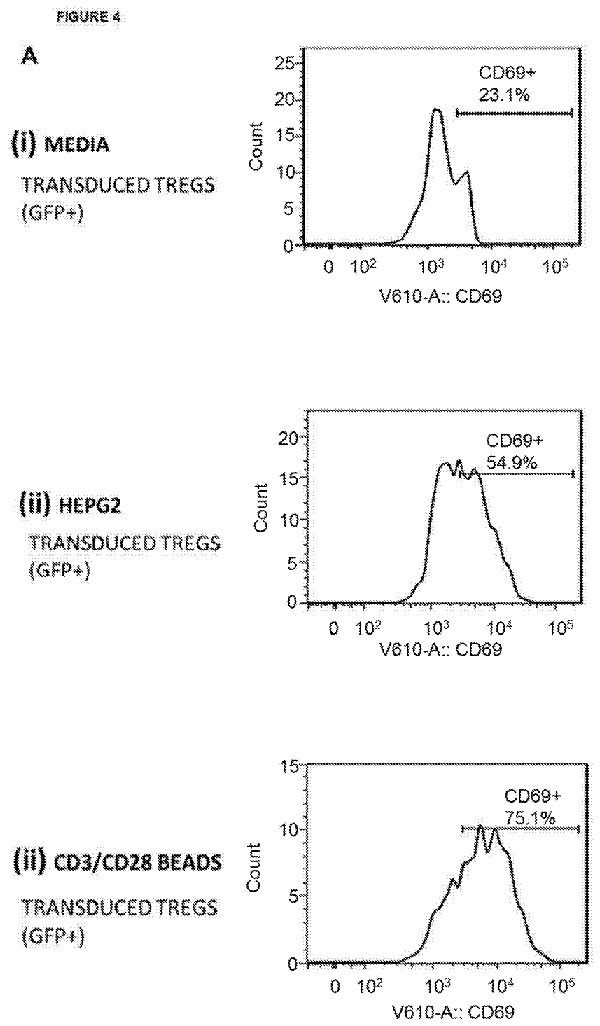

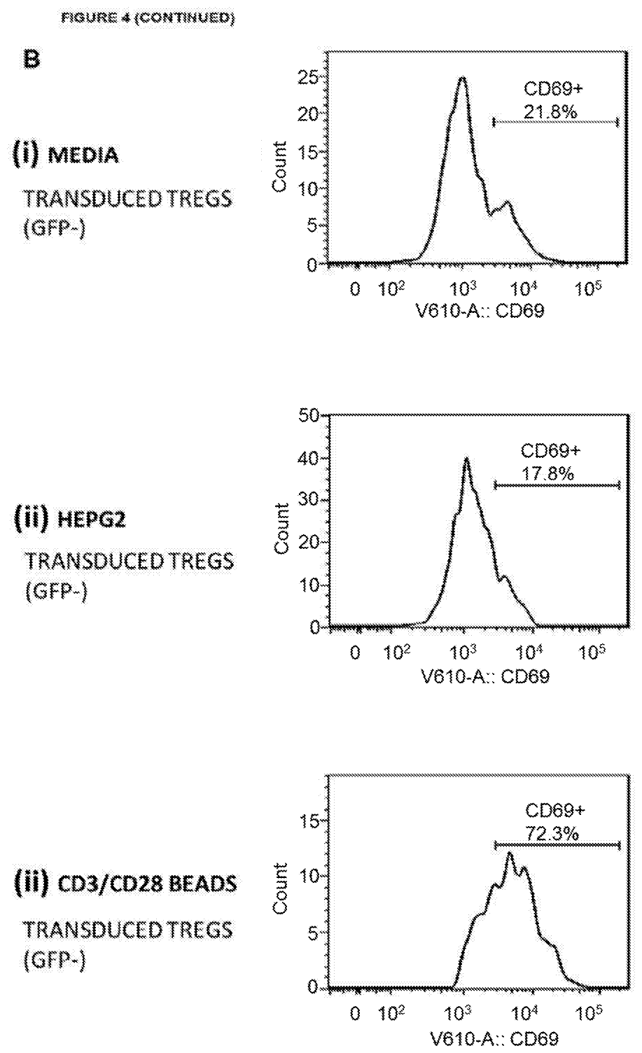

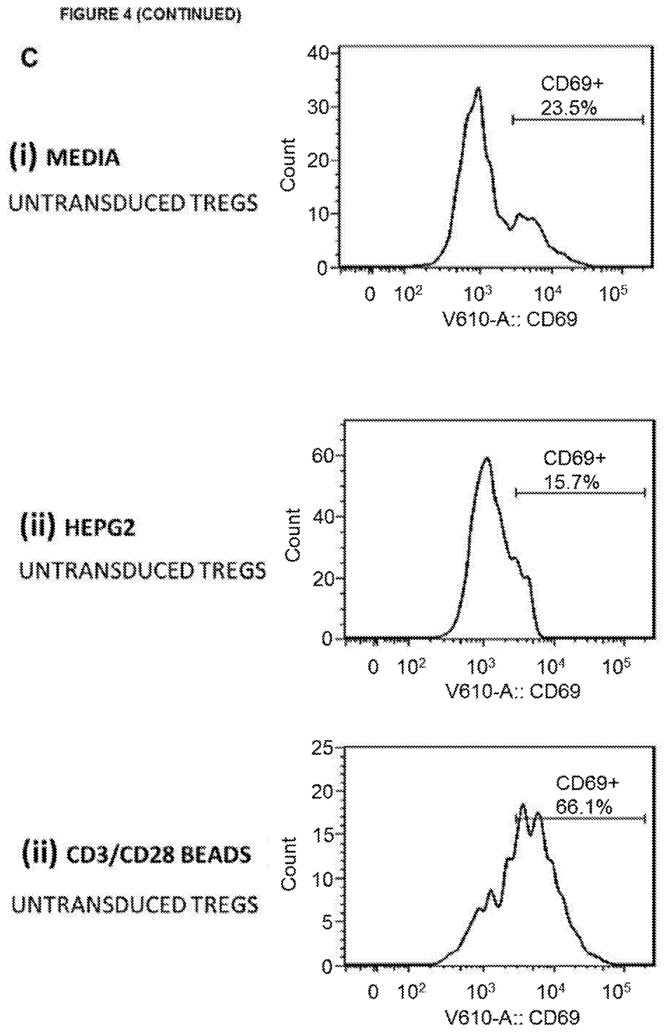

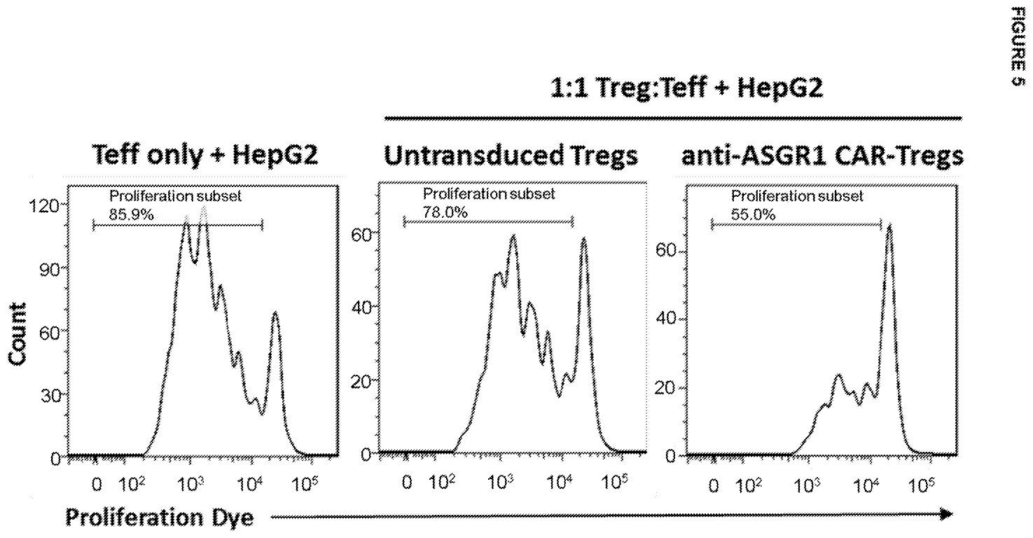

FIG. 4 shows that no upregulation of CD69 is observed in the presence of media alone. Following culture with HepG2 cells, only the GFP+ transduced Tregs (but not GPF-negative transduced cells or untransduced cells) upregulate CD69. In contrast, all cells upregulated CD69 when cultured with non-specific anti-CD3/CD28 bead stimulation.

### Example 5

### Evaluation of the Antigen-Specificity Suppressive Function of Anti-ASGR1 CAR-Tregs

To assess the capacity of anti-ASGR1 CAR Tregs to suppress the proliferation of activated effector CD4+CD25− T cells in an antigen specific manner, pre-activated CD4+CD25− effector T cells were co-cultured with anti-ASGR1 CAR-Tregs or untransduced Tregs in the presence of HepG2 cells.

Teff pre-activation was with the following conditions: CD4+CD25− T cells activated; aCD3/CD28 beads 1:10; 14 hours. Suppression assay conditions were as follows: 3 days culture; 100,000 Teff; 30,000 HepG2; untransduced vs anti-ASGR1 CAR-Tregs.

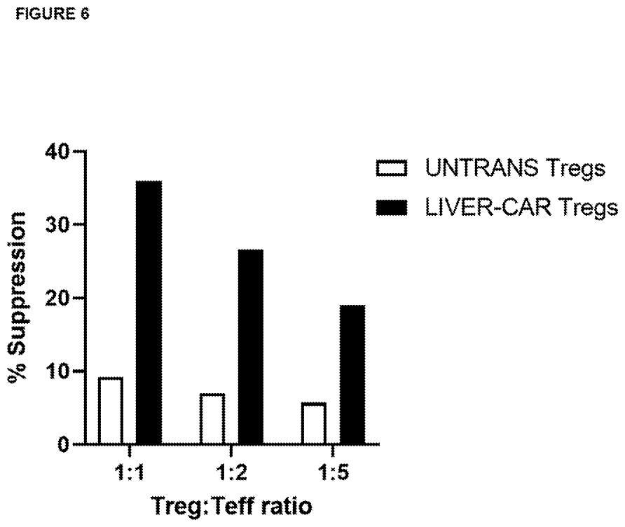

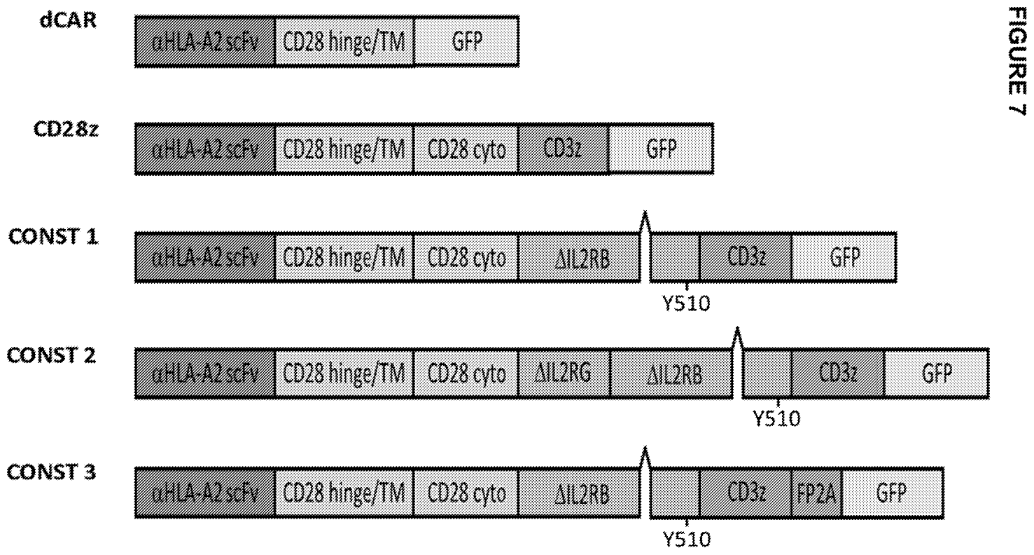

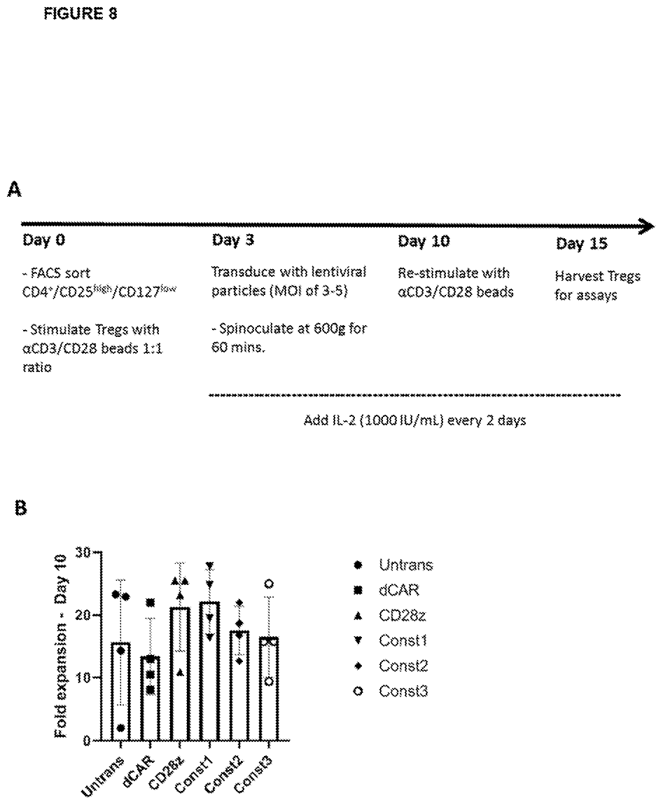

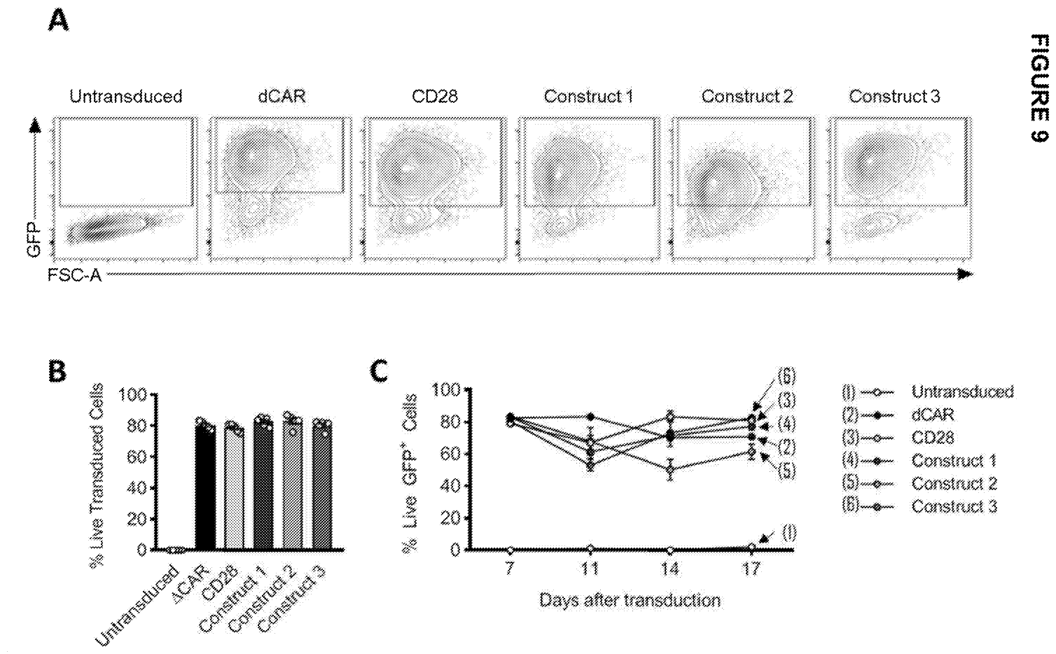

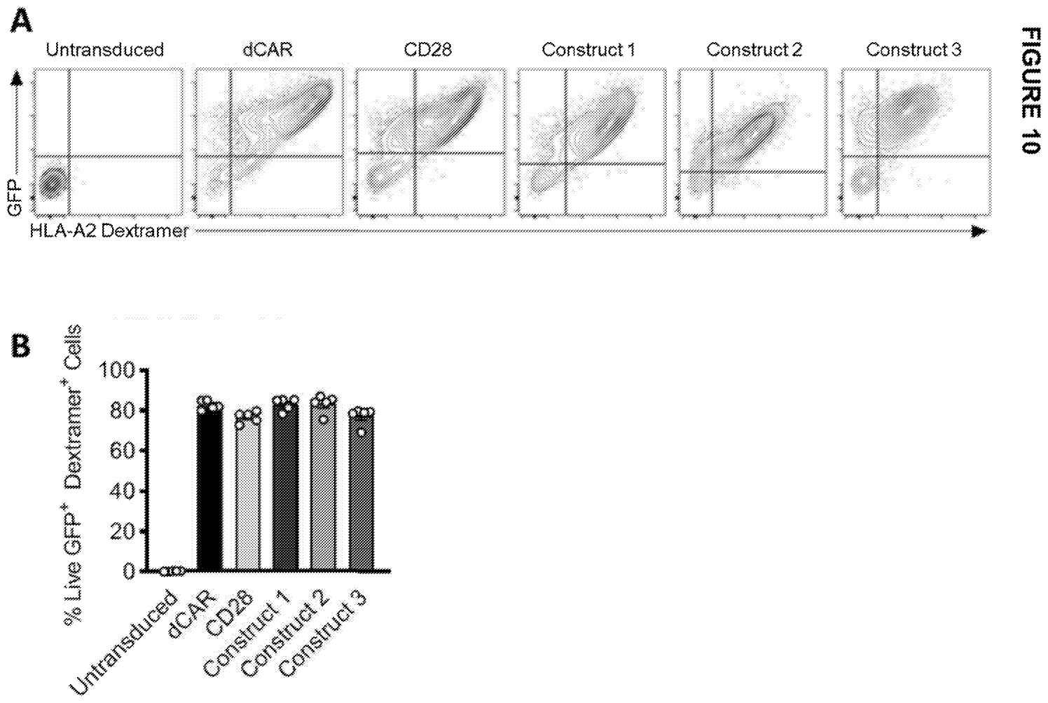

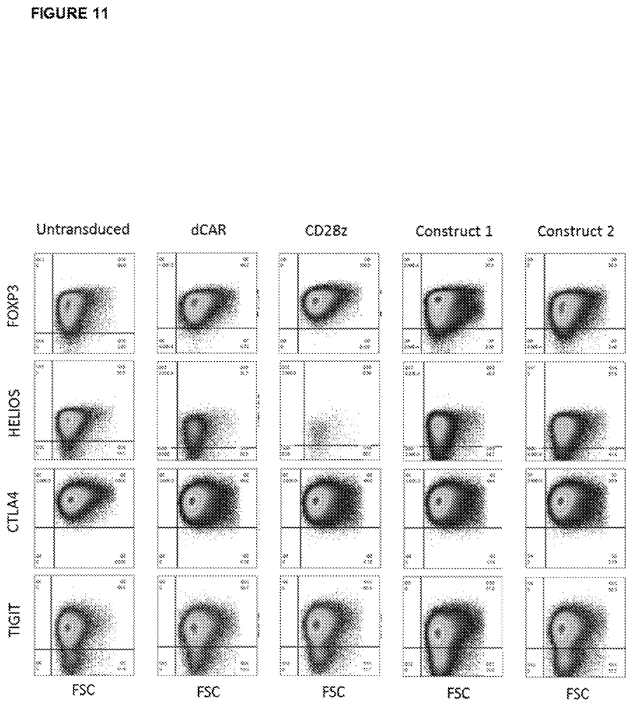

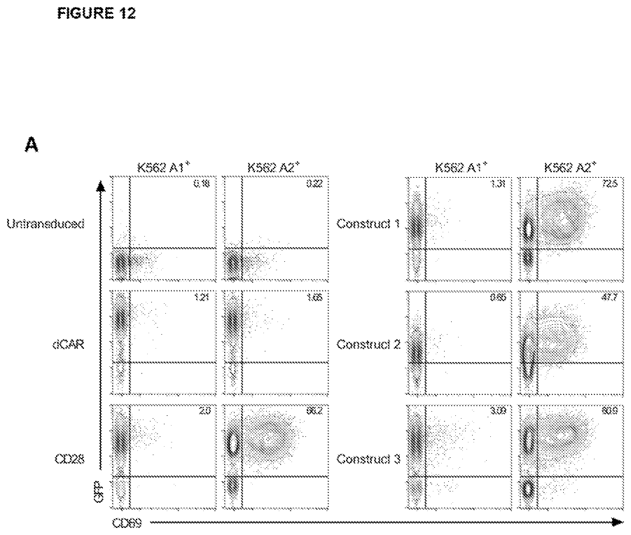

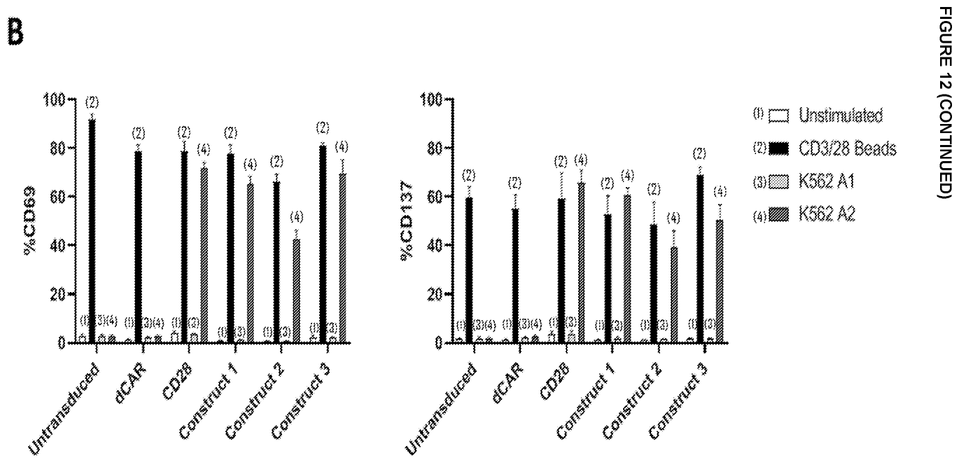

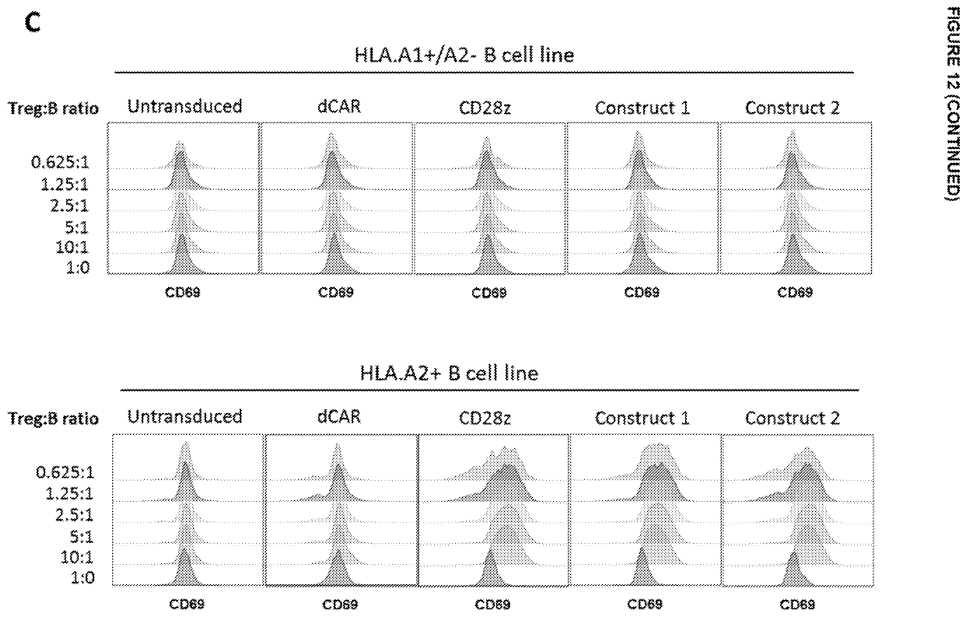

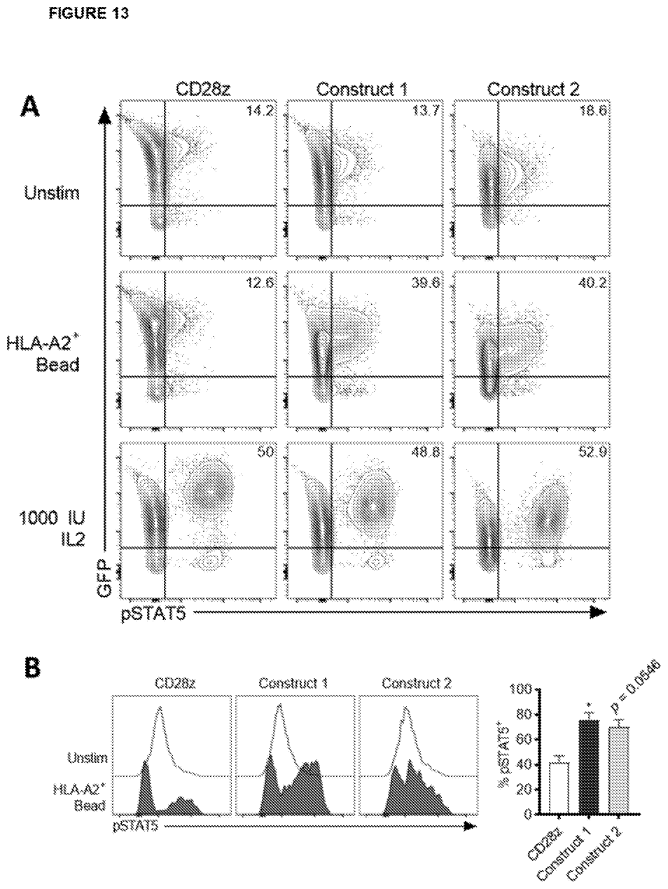

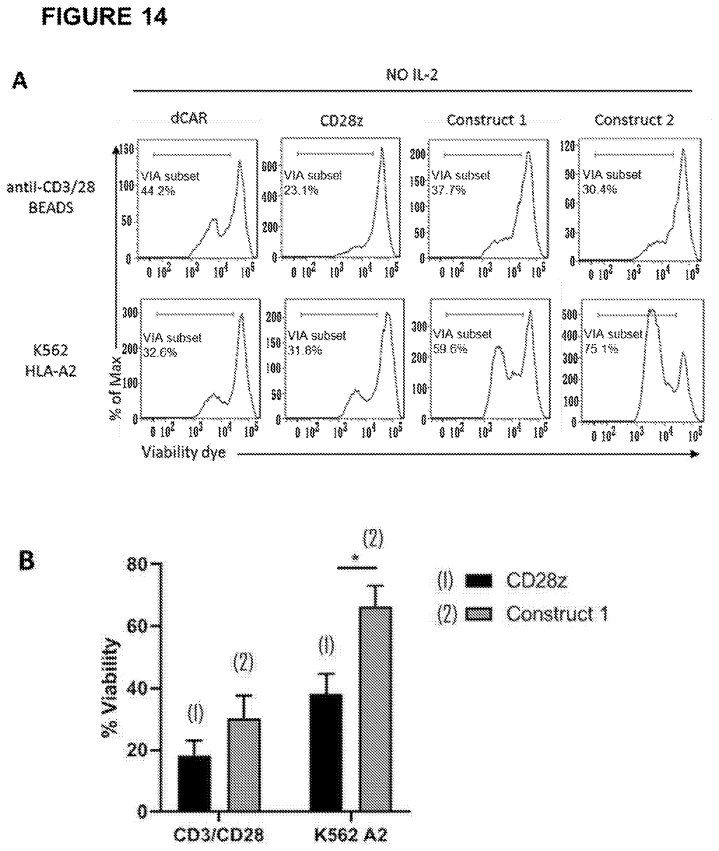

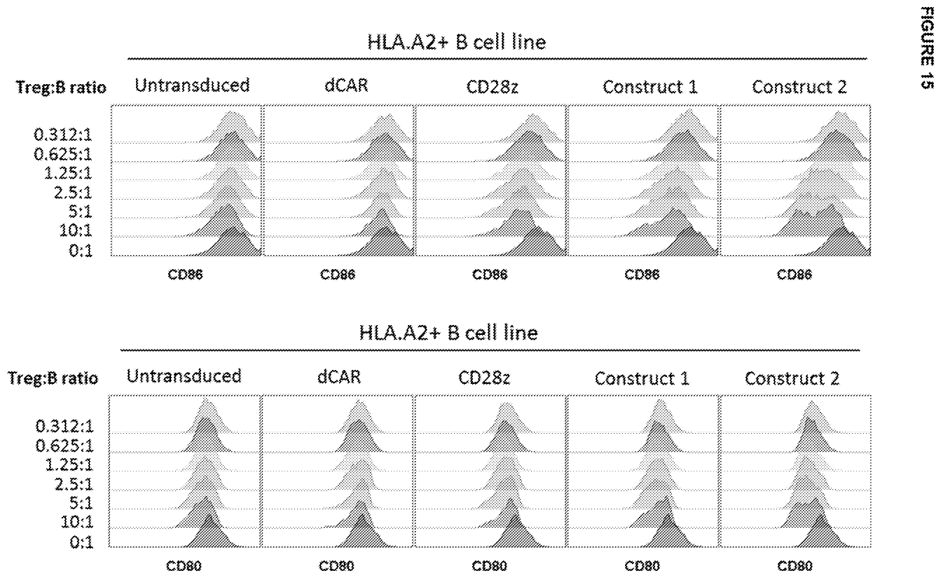

The suppression assay (FIG. 5 and FIG. 6) shows the impact of anti-ASGR1 CAR-Tregs on the proliferative capacity of effector T cells stained with Proliferation Dye and pre-activated with anti-CD3/CD28 beads. In response to HepG2 stimulation, anti-ASGR1 CAR Tregs were capable of effectively suppress conventional T cell proliferation at a lower ratio of Treg to T effector than untransduced Tregs.

### Example 6

### Generation of Anti-HLA.A2 IL2R CAR-Tregs

The efficacy of endodomains which comprise a STAT5 association motif and a JAK1- and/or a JAK2-binding motif (and optionally comprise a JAK3-binding motif and/or do not comprise a STAT3 association motif) is demonstrated in Examples 6-13 using HLA.A2-CAR constructs. Although the exemplified antigen-binding domain targets HLA.A2, the endodomains are considered to be broadly applicable, for example to CAR constructs comprising an antigen-binding domain which targets a liver-specific antigen, e.g. ASGR.

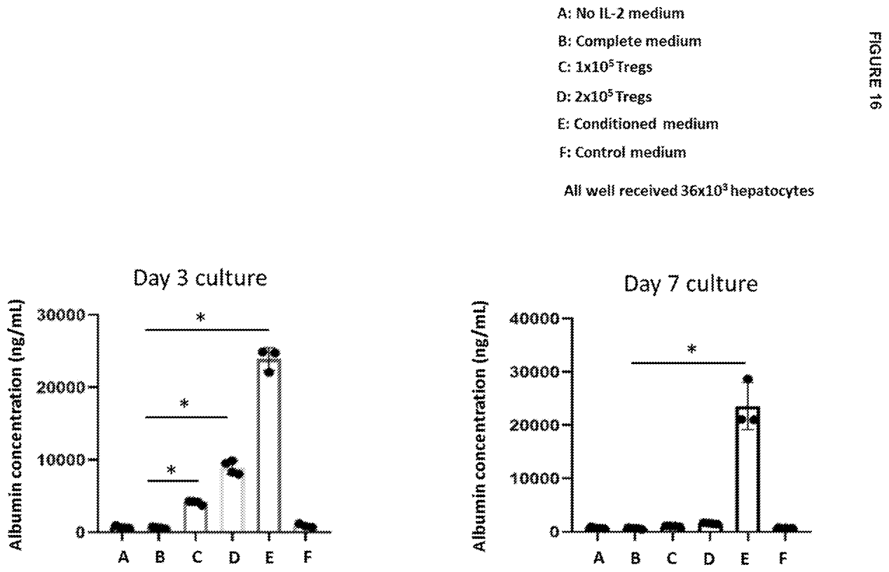

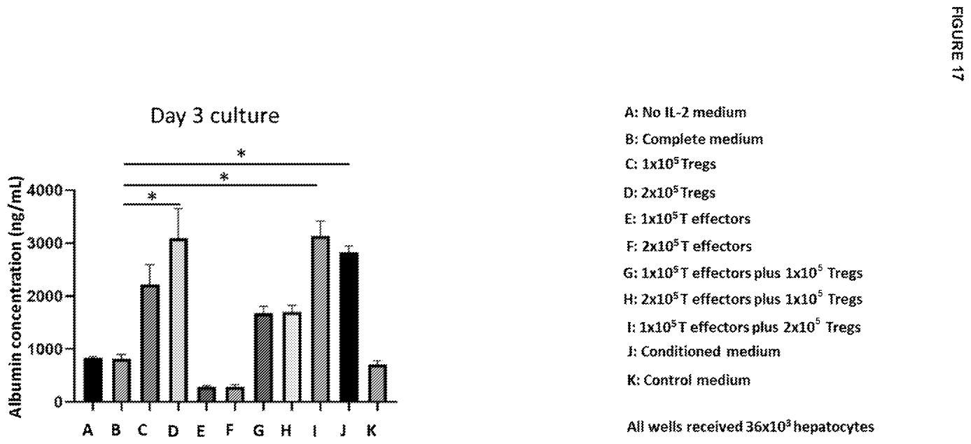

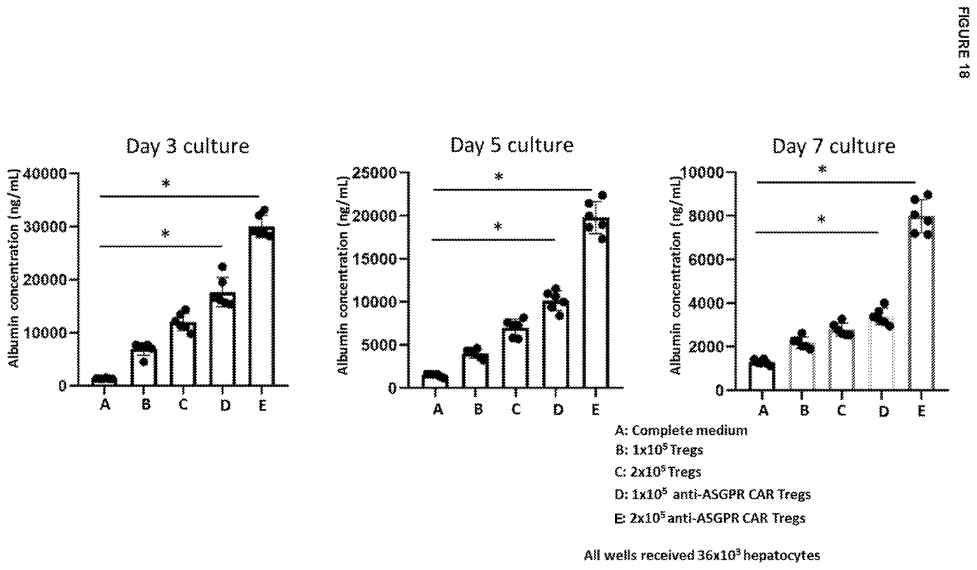

CD4+CD25hiCD127low cells were isolated and activated with anti-CD3/CD28 beads. Three days after activation Tregs were transduced with lentivirus containing the HLA.A2-CAR constructs (FIG. 7) and GFP reporter gene. Cellular expansion of total Tregs after polyclonal activation showed no significant differences between untransduced or transduced Treg (FIG. 8).

### Example 7

### Quantification of Transduction Efficacy of Anti-HLA.A2 IL2R Constructs Over Time

GFP expression was analysed on Tregs untransduced and transduced with CAR constructs at different time points after cell activation.

Frequency of GFP+ cells was analysed to evaluate the transduction efficacy and the expression persistence of the different constructs over the Treg expansion period. Tregs containing dCAR, CD28z, Construct 1, 2 and 3 showed similar expression frequencies after transduction. The percentages of GFP+ cells among whole Tregs were maintained during polyclonal cellular expansion (FIG. 9).

### Example 8

### Quantification of Cell Surface Expression of Anti-HLA.A2 IL2R CAR Constructs on Transduced Tregs

Membrane expression of CAR construct on untransduced and transduced Tregs was analysed by PE-conjugated HLA-A*0201/CINGVCWTV dextramers (Immudex, Copenhagen, Denmark). The frequency of Tregs expressing the CAR protein in the cell surface (HLA-A2 dextramer+) was similar between all the constructs (FIG. 10).

### Example 9

### Phenotypic Characterization of CAR Tregs After Polyclonal Cell Expansion

Tregs were cultured and expanded for 15 days in the presence of anti-CD3/CD28 activation beads and IL-2. Treg related markers FOXP3, HELIOS, CTLA4 and TIGIT were analysed by FACS on untransduced and transduced Tregs to assess phenotypic lineage stability on day 15 of culture.

Untransduced and CAR-transduced showed similar expression levels of proteins associated with Treg lineage and function after polyclonal expansion (FIG. 11).

### Example 10

### Evaluation of the Antigen-Specificity of Anti-HLA.A2 IL2R CAR Tregs

Untransduced and transduced Tregs were cultured for 18 hours in the presence of different stimulus. CD69 and CD137 activation markers were analysed to assess specific and unspecific cell activation.

Transduced Tregs with the CD28z, Construct 1, 2 and 3 CARs showed similar specificity for HLA-A2 molecules based on the expression of T cell activation markers. The expression of CD69 and CD137 was not increase on inactivated cells or after the culture with HLA-A1 expressing cells. The dCAR construct showed no activation due to the lack of signaling endodomains (FIG. 12).

### Example 11

### STAT5 Phosphorylation Analysis as an Indicator of IL2R CAR Signaling

Transduced CAR Tregs were rested overnight in culture media without IL2. STAT5 phosphorylation of Tregs was assessed by FACS analysis 10 and 120 minutes after culture with media alone, 1000 IU/ml IL-2 or in the presence of HLA.A2-Ig based artificial APCs (produced following the protocol described at DOI: 10.3791/2801).

The integration of the IL2R endodomains into the CAR construct showed efficient phosphorylation of STAT5 after the CAR activation by HLA-A2 molecules. No significant increase of pSTAT5 was detected on CAR-Tregs without the IL2R endodomains after culture with HLA-A2 beads (FIG. 13).

### Example 12

### Evaluation of Treg Survival After Unspecific and HLA.A2 Specific Activation in the Absence of IL-2

CAR transduced Tregs with different constructs were cultured with anti-CD3/28 activation beads and K562.A2 expression cells without the presence of IL-2. Cell survival was assessed 7 days after activation by FACS analysis.

Tregs expressing a CAR construct containing the IL2R endodomain showed increased cell viability compared to the reference CD28z after the cell culture with HLA-A2 expression cells. The differences were not observed after polyclonal activation of the Tregs demonstrating that the effect is dependent on CAR signalling (FIG. 14).

### Example 13

### Treg Suppression Potency Test: Evaluate the Immunoregulatory Function of Tregs by Analysing the Modulation of Co-Stimulatory Molecules on B Cells

B cell expression of CD80 and CD86 after co-culture with Tregs was analysed to evaluate the capacity of Tregs to reduce the expression of co-stimulatory molecules on antigen presenting cells.

Tregs expressing the CD28z, Construct 1 and Construct 2 CARs showed increased suppressive function compared to untransduce or dCAR expressing Tregs. CD80 and CD86 expression on B cells is only downregulated after culture with Tregs that signal through the CAR molecule (FIG. 15).

### Example 14

### Evaluation of the Effect of CAR Tregs on Albumin Production in Liver Cells

Methods:

Cryopreserved adult human primary hepatocytes (Lonza) were plated on Type-1 Collagen (rat tail derived) -coated plates at a cell density of 225 K/cm2 (36 K/well in half area 96 well plate or transwell plate) in Williams E medium (Gibco) supplemented with 10%FBS, 2 mM L-Glutamine, 1% ITS, 10 nM Hepes, 100 U/L Pen/Strep with/without 50 IU/mL of human recombinant interleukin-2 (Aldesleukin, Novartis). Once hepatocytes were attached 24 hours later, CD4+CD25+Foxp3+ regulatory T cells were added to the culture in the presence or absence of CD4+CD25− effector T cells. The supernatant was changed every 48 hours and cryopreserved for analysis. Indirect co-cultures were undertaken using a HTS Transwell membrane (pore size 0.4 um; CORNING). For some experiments regulatory T cells and effector T cells were pre-activated in the presence of anti-CD3 and anti-CD28 beads. For other experiments, non-preactivated regulatory T cells were lentivirally transduced with an anti-ASGPR1 CAR (construct 1 as described in Example 1) before being added to the culture plates. In some experiments hepatocytes were cultured with conditioned medium obtained from cultures containing expanding regulatory T cells. Albumin concentration, a marker of hepatocyte function and viability, was measured in the medium using a human albumin enzyme linked immunosorbent assay (ELISA—Bethyl Laboratories). For stastistical analysis we performed a Kruskal-Wallis non-parametric test with Dunn's inter-group post-tests. Asterisks denote p<0.05. Only pair-wise comparisons with hepatocytes alone and p<0.05 are shown. Plots shown are representative of 3 independent experiments.

RESULTS: To determine the capacity of anti-ASGPR CAR Tregs to mediate trophic and cytoprotective effects on liver cells in response to antigenic stimulation, we set up co-culture experiments in which primary human hepatocytes were cultured for up to 7 days with different combinations of Tregs and/or effector T cells. In order to assess the effects on hepatocyte phenotypic stability and function, we quantify the production of albumin into the supernatant which correlates with hepatocyte synthetic function, viability and lineage stability. The addition of Tregs previously activated in the presence of anti-CD3/anti-CD28 beads to the cultured primary hepatocytes resulted in increased albumin levels. This effect was significant after 3 days of co-culture but no longer at day 7, reflecting the need of Tregs to receive repeated antigen stimulation in order to exert the trophic effects on hepatocytes (FIG. 16). The beneficial effect of Tregs did not require cell-to-cell contact, given that it could be replicated by culturing hepatocytes with conditioned media obtained from activated Tregs (FIG. 17). Of note, the conditioned media, which was added to the culture plates repeatedly every 48 h induced a more persistent beneficial effect than pre-activated Tregs, which typically require weekly re-activations. In contrast to Tregs, effector T cells were not capable of increasing albumin levels (FIG. 17).

The use of resting (not preactivated) Tregs exerted a weaker non-significant effects (FIG. 18). A significant effect was only achieved when the resting Tregs expressed an anti-ASGPR CAR capable of recognizing ASGPR on the cultured hepatocytes and inducing Treg activation (FIG. 18).

These results show that regulatory T cells (Tregs) are capable of providing trophic cytoprotective effects to liver cells. This requires Treg activation and can occur in the absence of cell-to-cell contact. The activation of Tregs can be achieved by stimulating them with conventional T cell stimulators such as anti-CD3/anti-CD28 beads or by transducing them with a CAR capable of inducing T cell activation after recognizing a hepatocyte-specific antigen such as ASGPR.

All publications mentioned in the above specification are herein incorporated by reference. Various modifications and variations of the disclosed methods, cells, compositions and uses of the invention will be apparent to the skilled person without departing from the scope and spirit of the invention. Although the invention has been disclosed in connection with specific preferred embodiments, it should be understood that the invention as claimed should not be unduly limited to such specific embodiments. Indeed, various modifications of the disclosed modes for carrying out the invention, which are obvious to the skilled person are intended to be within the scope of the following claims.

1. An engineered regulatory T cell (Treg) comprising a chimeric antigen receptor (CAR), wherein the CAR comprises an antigen recognition domain which specifically binds to asialoglycoprotein receptor (ASGR).

2. The engineered Treg according to claim 1, wherein the antigen recognition domain specifically binds to ASGR1 and/or ASGR2.

3. The engineered Treg according to claim 1, wherein the antigen recognition domain binds to human ASGR.

4. The engineered Treg according to claim 1, wherein the antigen recognition domain binds to one or more polypeptides selected from Human ASGR1 isoform a, Human ASGR1 isoform b, Human ASGR2 isoform a, Human ASGR2 isoform b, Human ASGR2 isoform c, and Human ASGR2 isoform d.

5. The engineered Treg according to claim 1, wherein the antigen recognition domain binds to ASGR with an affinity of about 1 pM to about 100 nM.

6. The engineered Treg according to claim 1, wherein the antigen recognition domain is an antibody, an antibody fragment, or derived from an antibody.

7. The engineered Treg according to claim 1, wherein the antigen recognition domain is an antigen-binding fragment (Fab), a single chain antibody (scFv), or a single-domain antibody (sdAb).

8. The engineered Treg according to claim 6, wherein the antigen recognition domain comprises one or more CDR regions selected from SEQ ID NOs: 11-73 or derivatives thereof comprising three or fewer amino acid substitutions.

9. The engineered Treg according to claim 6, wherein the antigen recognition domain comprises CDR1, CDR2 and CDR3 regions comprising: SEQ ID NOs: 11, 12 and 13, respectively; SEQ ID NOs: 14, 15 and 16, respectively; SEQ ID NOs: 17, 18 and 19, respectively; SEQ ID NOs: 20, 21 and 22, respectively; SEQ ID NOs: 23, 24 and 25, respectively; SEQ ID NOs: 26, 27 and 28, respectively; or SEQ ID NOs: 29, 30 and 31, respectively.

10. The engineered Treg according to claim 6, wherein the antigen recognition domain comprises CDR1, CDR2 and CDR3 regions comprising:
(i) SEQ ID NOs: 11, 12 and 13, respectively; and SEQ ID NOs: 23, 24 and 25, respectively; or SEQ ID NOs: 26, 27 and 28, respectively; or SEQ ID NOs: 29, 30 and 31, respectively;
(ii) SEQ ID NOs: 14, 15 and 16, respectively; and SEQ ID NOs: 23, 24 and 25, respectively; or SEQ ID NOs: 26, 27 and 28, respectively; or SEQ ID NOs: 29, 30 and 31, respectively;
(iii) SEQ ID NOs: 17, 18 and 19, respectively; and SEQ ID NOs: 23, 24 and 25, respectively; or SEQ ID NOs: 26, 27 and 28, respectively; or SEQ ID NOs: 29, 30 and 31, respectively.

11. The engineered Treg according to claim 1, wherein the antigen recognition domain comprises an amino acid sequence which has at least about 90% identity to one or more of SEQ ID NOs: 74-80.

12. The engineered Treg according to claim 1, wherein the CAR comprises a transmembrane (TM) domain and an intracellular signaling domain, and wherein the CAR comprises a hinge domain and/or one or more co-stimulatory domains.

13. The engineered Treg according to claim 1, wherein the CAR comprises one or more hinge domains selected from the group consisting of a CD28 hinge domain, a CD8α hinge domain, an IgG hinge domain, and an IgD hinge domain.

14. The engineered Treg according to claim 1, wherein the CAR comprises one or more TM domains selected from the group consisting of a CD28 TM domain, an ICOS TM domain, a CD8α TM domain, a CD4 TM domain, an OX40 TM domain, a 4-1BB TM domain, and a CD3 zeta TM domain.

15. The engineered Treg according to claim 1, wherein the CAR comprises one or more co-stimulatory domains selected from the group consisting of a CD28 signaling domain, an ICOS signaling domain, an OX40 signaling domain, a 4-1BB signaling domain, a CD27 signaling domain, and a TNFRSF25 signaling domain.

16. The engineered Treg according to claim 1, wherein the CAR comprises one or more intracellular signaling domains selected from the group consisting of the CD3 zeta signalling domain or any of its homologs, a CD3 polypeptide, a syk family tyrosine kinase, a src family tyrosine kinase, CD2, CD5, and CD8.

17. The engineered Treg according to claim 1, wherein the CAR comprises: a CD8α or CD28 hinge domain; a CD28 TM domain; a CD28 signaling domain; and the CD3 zeta signaling domain.

18. The engineered Treg according to claim 1, wherein the CAR comprises a signal peptide and/or a reporter peptide linked by a self-cleaving or cleavage domain.

19. The engineered Treg according to claim 1, wherein the CAR comprises an amino acid sequence which has at least 90% identity to one or more of SEQ ID NOs: 149-152 or 167-169.

20. The engineered Treg according to claim 1, wherein the CAR comprises an endodomain which comprises a STAT5 association motif and a JAK1- and/or a JAK2-binding motif.

21. The engineered Treg according to claim 20, wherein the endodomain comprises a JAK3-binding motif.

22. The engineered Treg according to claim 20, wherein the endodomain does not comprise a STAT5 association motif.

23. The engineered Treg according to claim 20, wherein the endodomain does not comprise the amino acid sequence YXXQ (SEQ ID NO: 133).

24. The engineered Treg according to claim 1, wherein the CAR does not comprise an endodomain which comprises a STAT5 association motif, a JAK-1 binding motif, and/or a JAK-2 binding motif.

25. The engineered Treg according to claim 1, wherein the Treg is a CD4+CD25+CD127− T cell and/or a CD4+CD25+FOXP3+ T cell.

26. The engineered Treg according to claim 1, wherein the Treg comprises an exogenous polynucleotide encoding a FOXP3 polypeptide.

27. A pharmaceutical composition comprising an engineered Treg according to claim 1.

28. (canceled)

29. A method of inducing tolerance to a liver transplant in a subject, or treating and/or preventing liver transplant rejection, liver graft-versus-host disease (GvHD), an autoimmune liver disease, or an inflammatory liver disorder in a subject, which comprises the step of administering to the subject an engineered Treg according to claim 1.

30. The method of claim 29, wherein the method is treating and/or preventing the autoimmune liver disease, and wherein the autoimmune liver disease is primary biliary cholangitis and/or primary sclerosing cholangitis.

31. The method of claim 29, wherein the method is treating and/or preventing the inflammatory liver disorder, and wherein the liver inflammatory disorder is liver cirrhosis, acute liver failure or acute-on-chronic liver failure; and/or wherein the liver inflammation is caused by alcohol, viral hepatitis, steatohepatitis, ischemia or drug toxicity or wherein the liver inflammation has no identifiable cause, wherein the subject is a mammal.

32.-55. (canceled)


SEQUENCE LISTING (175 sequences)

SEQ ID NO: 1 - protein, 291 aa, Homo sapiens
MTKEYQDLQHLDNEESDHHQLRKGPPPPQPLLQRLCSGPRLLLLSLGLSLLLLVVVCVIGSQNSQLQEELRGLRETFSNFTASTEAQVKGLSTQGGNVGRKMKSLESQLEKQQKDLSEDHSSLLLHVKQFVSDLRSLSCQMAALQGNGSERTCCPVNWVEHERSCYWFSRSGKAWADADNYCRLEDAHLVVVTSWEEQKFVQHHIGPVNTWMGLHDQNGPWKWVDGTDYETGFKNWRPEQPDDWYGHGLGGGEDCAHFTDDGRWNDDVCQRPYRWVCETELDKASQEPPLL

SEQ ID NO: 2 - protein, 252 aa, Homo sapiens
MTKEYQDLQHLDNEESDHHQLRKDSQLQEELRGLRETFSNFTASTEAQVKGLSTQGGNVGRKMKSLESQLEKQQKDLSEDHSSLLLHVKQFVSDLRSLSCQMAALQGNGSERTCCPVNWVEHERSCYWFSRSGKAWADADNYCRLEDAHLVVVTSWEEQKFVQHHIGPVNTWMGLHDQNGPWKWVDGTDYETGFKNWRPEQPDDWYGHGLGGGEDCAHFTDDGRWNDDVCQRPYRWVCETELDKASQEPPLL

SEQ ID NO: 3 - protein, 311 aa, Homo sapiens
MAKDFQDIQQLSSEENDHPFHQGEGPGTRRLNPRRGNPFLKGPPPAQPLAQRLCSMVCFSLLALSFNILLLVVICVTGSQSEGHRGAQLQAELRSLKEAFSNFSSSTLTEVQAISTHGGSVGDKITSLGAKLEKQQQDLKADHDALLFHLKHFPVDLRFVACQMELLHSNGSQRTCCPVNWVEHQGSCYWFSHSGKAWAEAEKYCQLENAHLVVINSWEEQKFIVQHTNPFNTWIGLTDSDGSWKWVDGTDYRHNYKNWAVTQPDNWHGHELGGSEDCVEVQPDGRWNDDFCLQVYRWVCEKRRNATGEVA

SEQ ID NO: 4 - protein, 287 aa, Homo sapiens
MAKDFQDIQQLSSEENDHPFHQGPPPAQPLAQRLCSMVCFSLLALSFNILLLVVICVTGSQSAQLQAELRSLKEAFSNFSSSTLTEVQAISTHGGSVGDKITSLGAKLEKQQQDLKADHDALLFHLKHFPVDLRFVACQMELLHSNGSQRTCCPVNWVEHQGSCYWFSHSGKAWAEAEKYCQLENAHLVVINSWEEQKFIVQHTNPFNTWIGLTDSDGSWKWVDGTDYRHNYKNWAVTQPDNWHGHELGGSEDCVEVQPDGRWNDDFCLQVYRWVCEKRRNATGEVA

SEQ ID NO: 5 - protein, 292 aa, Homo sapiens
MAKDFQDIQQLSSEENDHPFHQGPPPAQPLAQRLCSMVCFSLLALSFNILLLVVICVTGSQSEGHRGAQLQAELRSLKEAFSNFSSSTLTEVQAISTHGGSVGDKITSLGAKLEKQQQDLKADHDALLFHLKHFPVDLRFVACQMELLHSNGSQRTCCPVNWVEHQGSCYWFSHSGKAWAEAEKYCQLENAHLVVINSWEEQKFIVQHTNPFNTWIGLTDSDGSWKWVDGTDYRHNYKNWAVTQPDNWHGHELGGSEDCVEVQPDGRWNDDFCLQVYRWVCEKRRNATGEVA

SEQ ID NO: 6 - protein, 306 aa, Homo sapiens
MAKDFQDIQQLSSEENDHPFHQGEGPGTRRLNPRRGNPFLKGPPPAQPLAQRLCSMVCFSLLALSFNILLLVVICVTGSQSAQLQAELRSLKEAFSNFSSSTLTEVQAISTHGGSVGDKITSLGAKLEKQQQDLKADHDALLFHLKHFPVDLRFVACQMELLHSNGSQRTCCPVNWVEHQGSCYWFSHSGKAWAEAEKYCQLENAHLVVINSWEEQKFIVQHTNPFNTWIGLTDSDGSWKWVDGTDYRHNYKNWAVTQPDNWHGHELGGSEDCVEVQPDGRWNDDFCLQVYRWVCEKRRNATGEVA

SEQ ID NO: 7 - protein, 284 aa, Mus musculus
MTKDYQDFQHLDNDNDHHQLRRGPPPTPRLLQRLCSGSRLLLLSSSLSILLLVVVCVITSQNSQLREDLLALRQNFSNLTVSTEDQVKALSTQGSSVGRKMKLVESKLEKQQKDLTEDHSSLLLHVKQLVSDVRSLSCQMAAFRGNGSERTCCPINWVEYEGSCYWFSSSVRPWTEADKYCQLENAHLVVVTSRDEQNFLQRHMGPLNTWIGLTDQNGPWKWVDGTDYETGFQNWRPEQPDNWYGHGLGGGEDCAHFTTDGRWNDDVCRRPYRWVCETKLDKAN

SEQ ID NO: 8 - protein, 255 aa, Mus musculus
MTKDYQDFQHLDNDNDHHQLRRGPPPTPRLLQRLCSGSRLLLLSSSLSILLLVVVCVITSQNSQLREDLLALRQNFSNLTVSTEDQVKALSTQGSSVGRKMKLVESKLEKQQKDLTEGSERTCCPINWVEYEGSCYWFSSSVRPWTEADKYCQLENAHLVVVTSRDEQNFLQRHMGPLNTWIGLTDQNGPWKWVDGTDYETGFQNWRPEQPDNWYGHGLGGGEDCAHFTTDGRWNDDVCRRPYRWVCETKLDKAN

SEQ ID NO: 9 - protein, 301 aa, Mus musculus
MEKDCQDIQQLDSEENDHQLSGDDEHGSHVQDPRIENPHWKGQPLSRPFPQRLCSTFRLSLLALAFNILLLVVICVVSSQSIQLQEEFRTLKETFSNFSSSTLMEFGALDTLGGSTNAILTSWLAQLEEKQQQLKADHSTLLFHLKHFPMDLRTLTCQLAYFQSNGTECCPVNWVEFGGSCYWFSRDGLTWAEADQYCQLENAHLLVINSREEQDFVVKHRSQFHIWIGLTDRDGSWKWVDGTDYRSNYRNWAFTQPDNWQGHEQGGGEDCAEILSDGHWNDNFCQQVNRWVCEKRRNITH

SEQ ID NO: 10 - protein, 198 aa, Mus musculus
MEFGALDTLGGSTNAILTSWLAQLEEKQQQLKADHSTLLFHLKHFPMDLRTLTCQLAYFQSNGTECCPVNWVEFGGSCYWFSRDGLTWAEADQYCQLENAHLLVINSREEQDFVVKHRSQFHIWIGLTDRDGSWKWVDGTDYRSNYRNWAFTQPDNWQGHEQGGGEDCAEILSDGHWNDNFCQQVNRWVCEKRRNITH

SEQ ID NO: 11 - protein, 6 aa, Artificial Sequence
  Description of sequence component of an antibody which targets the ASGR antigen
EKYAMA

SEQ ID NO: 12 - protein, 8 aa, Artificial Sequence
  Description of sequence component of an antibody which targets the ASGR antigen
RISARGVT

SEQ ID NO: 13 - protein, 11 aa, Artificial Sequence
  Description of sequence component of an antibody which targets the ASGR antigen
HKRHEHTRFDS

SEQ ID NO: 14 - protein, 5 aa, Artificial Sequence
  Description of sequence component of an antibody which targets the ASGR antigen
RYTMG

SEQ ID NO: 15 - protein, 17 aa, Artificial Sequence
  Description of sequence component of an antibody which targets the ASGR antigen
AIGPPGSNTYYADSVKG

SEQ ID NO: 16 - protein, 10 aa, Artificial Sequence
  Description of sequence component of an antibody which targets the ASGR antigen
WVMLRGRFDY

SEQ ID NO: 17 - protein, 5 aa, Artificial Sequence
  Description of sequence component of an antibody which targets the ASGR antigen
DYGMG

SEQ ID NO: 18 - protein, 17 aa, Artificial Sequence
  Description of sequence component of an antibody which targets the ASGR antigen
AIGRNGSQTYYADSVKG

SEQ ID NO: 19 - protein, 14 aa, Artificial Sequence
  Description of sequence component of an antibody which targets the ASGR antigen
LRRGRGLNTFTLDY

SEQ ID NO: 20 - protein, 5 aa, Artificial Sequence
  Description of sequence component of an antibody which targets the ASGR antigen
AAGMG

SEQ ID NO: 21 - protein, 17 aa, Artificial Sequence
  Description of sequence component of an antibody which targets the ASGR antigen
AIGRNGSQTYYADSVKG

SEQ ID NO: 22 - protein, 14 aa, Artificial Sequence
  Description of sequence component of an antibody which targets the ASGR antigen
LRRGRGLNTFTLDY

SEQ ID NO: 23 - protein, 11 aa, Artificial Sequence
  Description of sequence component of an antibody which targets the ASGR antigen
RASQAIGRWLL

SEQ ID NO: 24 - protein, 7 aa, Artificial Sequence
  Description of sequence component of an antibody which targets the ASGR antigen
PGSRLRS

SEQ ID NO: 25 - protein, 9 aa, Artificial Sequence
  Description of sequence component of an antibody which targets the ASGR antigen
QQAYAWPPT

SEQ ID NO: 26 - protein, 11 aa, Artificial Sequence
  Description of sequence component of an antibody which targets the ASGR antigen
RASQAIGRWLL

SEQ ID NO: 27 - protein, 7 aa, Artificial Sequence
  Description of sequence component of an antibody which targets the ASGR antigen
PGSRLQS

SEQ ID NO: 28 - protein, 9 aa, Artificial Sequence
  Description of sequence component of an antibody which targets the ASGR antigen
QQAYQLPVT

SEQ ID NO: 29 - protein, 11 aa, Artificial Sequence
  Description of sequence component of an antibody which targets the ASGR antigen
RASQAIGRWLL

SEQ ID NO: 30 - protein, 7 aa, Artificial Sequence
  Description of sequence component of an antibody which targets the ASGR antigen
PGSRLQS

SEQ ID NO: 31 - protein, 9 aa, Artificial Sequence
  Description of sequence component of an antibody which targets the ASGR antigen
QQAYSLPPT

SEQ ID NO: 32 - protein, 11 aa, Artificial Sequence
  Description of sequence component of an antibody which targets the ASGR antigen
RASGDIGHALW

SEQ ID NO: 33 - protein, 8 aa, Artificial Sequence
  Description of sequence component of an antibody which targets the ASGR antigen
RGGSALQS

SEQ ID NO: 34 - protein, 9 aa, Artificial Sequence
  Description of sequence component of an antibody which targets the ASGR antigen
GQSHVRPFT

SEQ ID NO: 35 - protein, 11 aa, Artificial Sequence
  Description of sequence component of an antibody which targets the ASGR antigen
QASKNIGERLV

SEQ ID NO: 36 - protein, 8 aa, Artificial Sequence
  Description of sequence component of an antibody which targets the ASGR antigen
GFASLLQS

SEQ ID NO: 37 - protein, 9 aa, Artificial Sequence
  Description of sequence component of an antibody which targets the ASGR antigen
GQYRWVPAT

SEQ ID NO: 38 - protein, 6 aa, Artificial Sequence
  Description of sequence component of an antibody which targets the ASGR antigen
STYPMH

SEQ ID NO: 39 - protein, 8 aa, Artificial Sequence
  Description of sequence component of an antibody which targets the ASGR antigen
SISPSGDS

SEQ ID NO: 40 - protein, 7 aa, Artificial Sequence
  Description of sequence component of an antibody which targets the ASGR antigen
NALRFDY

SEQ ID NO: 41 - protein, 6 aa, Artificial Sequence
  Description of sequence component of an antibody which targets the ASGR antigen
KPYAMH

SEQ ID NO: 42 - protein, 8 aa, Artificial Sequence
  Description of sequence component of an antibody which targets the ASGR antigen
SISSTGLS

SEQ ID NO: 43 - protein, 11 aa, Artificial Sequence
  Description of sequence component of an antibody which targets the ASGR antigen
DASRFRQPFDY

SEQ ID NO: 44 - protein, 6 aa, Artificial Sequence
  Description of sequence component of an antibody which targets the ASGR antigen
PKYGMA

SEQ ID NO: 45 - protein, 8 aa, Artificial Sequence
  Description of sequence component of an antibody which targets the ASGR antigen
RIGATGSE

SEQ ID NO: 46 - protein, 12 aa, Artificial Sequence
  Description of sequence component of an antibody which targets the ASGR antigen
HRGTAHSSFFDY

SEQ ID NO: 47 - protein, 6 aa, Artificial Sequence
  Description of sequence component of an antibody which targets the ASGR antigen
SANGMH

SEQ ID NO: 48 - protein, 8 aa, Artificial Sequence
  Description of sequence component of an antibody which targets the ASGR antigen
VISATGDQ

SEQ ID NO: 49 - protein, 11 aa, Artificial Sequence
  Description of sequence component of an antibody which targets the ASGR antigen
GYDRRHRKFDY

SEQ ID NO: 50 - protein, 6 aa, Artificial Sequence
  Description of sequence component of an antibody which targets the ASGR antigen
ADYSMY

SEQ ID NO: 51 - protein, 8 aa, Artificial Sequence
  Description of sequence component of an antibody which targets the ASGR antigen
DISPSGSM

SEQ ID NO: 52 - protein, 13 aa, Artificial Sequence
  Description of sequence component of an antibody which targets the ASGR antigen
GLPGQNMHVGFDY

SEQ ID NO: 53 - protein, 11 aa, Artificial Sequence
  Description of sequence component of an antibody which targets the ASGR antigen
RASQAIGRWLL

SEQ ID NO: 54 - protein, 8 aa, Artificial Sequence
  Description of sequence component of an antibody which targets the ASGR antigen
YAASRLQS

SEQ ID NO: 55 - protein, 9 aa, Artificial Sequence
  Description of sequence component of an antibody which targets the ASGR antigen
QQAYSLPPT

SEQ ID NO: 56 - protein, 11 aa, Artificial Sequence
  Description of sequence component of an antibody which targets the ASGR antigen
RASMSIDESLW

SEQ ID NO: 57 - protein, 8 aa, Artificial Sequence
  Description of sequence component of an antibody which targets the ASGR antigen
RGGSGLQS

SEQ ID NO: 58 - protein, 9 aa, Artificial Sequence
  Description of sequence component of an antibody which targets the ASGR antigen
GQAARRPYT

SEQ ID NO: 59 - protein, 11 aa, Artificial Sequence
  Description of sequence component of an antibody which targets the ASGR antigen
RASHYIGNELW

SEQ ID NO: 60 - protein, 8 aa, Artificial Sequence
  Description of sequence component of an antibody which targets the ASGR antigen
RRGSGLQS

SEQ ID NO: 61 - protein, 9 aa, Artificial Sequence
  Description of sequence component of an antibody which targets the ASGR antigen
GQARHRPYT

SEQ ID NO: 62 - protein, 11 aa, Artificial Sequence
  Description of sequence component of an antibody which targets the ASGR antigen
RASSNIGRSLV

SEQ ID NO: 63 - protein, 8 aa, Artificial Sequence
  Description of sequence component of an antibody which targets the ASGR antigen
AGGSLLQS

SEQ ID NO: 64 - protein, 9 aa, Artificial Sequence
  Description of sequence component of an antibody which targets the ASGR antigen
GQYAEEPFT

SEQ ID NO: 65 - protein, 11 aa, Artificial Sequence
  Description of sequence component of an antibody which targets the ASGR antigen
RASVKIGERLW

SEQ ID NO: 66 - protein, 8 aa, Artificial Sequence
  Description of sequence component of an antibody which targets the ASGR antigen
RDASLLQS

SEQ ID NO: 67 - protein, 9 aa, Artificial Sequence
  Description of sequence component of an antibody which targets the ASGR antigen
GQSWMRPYT

SEQ ID NO: 68 - protein, 11 aa, Artificial Sequence
  Description of sequence component of an antibody which targets the ASGR antigen
RASSYIGGELW

SEQ ID NO: 69 - protein, 8 aa, Artificial Sequence
  Description of sequence component of an antibody which targets the ASGR antigen
SGTSGLQS

SEQ ID NO: 70 - protein, 9 aa, Artificial Sequence
  Description of sequence component of an antibody which targets the ASGR antigen
GQAAKRPFT

SEQ ID NO: 71 - protein, 11 aa, Artificial Sequence
  Description of sequence component of an antibody which targets the ASGR antigen
RASSWINSDLV

SEQ ID NO: 72 - protein, 8 aa, Artificial Sequence
  Description of sequence component of an antibody which targets the ASGR antigen
AGGSLLQS

SEQ ID NO: 73 - protein, 9 aa, Artificial Sequence
  Description of sequence component of an antibody which targets the ASGR antigen
GQYLEEPYT

SEQ ID NO: 74 - protein, 120 aa, Artificial Sequence
  Description of sequence component of an antibody which targets the ASGR antigen
EVQLLESGGGLVQPGGSLRLSCAASGFTFEKYAMAWVRQAPGKGLEWVSRISARGVTTYYADSVKGRFTISRDNSKNTLYLQMNSLRAEDTAVYYCAKHKRHEHTRFDSWGQGTLVTVSS

SEQ ID NO: 75 - protein, 119 aa, Artificial Sequence
  Description of sequence component of an antibody which targets the ASGR antigen
ELQLLEFGGGLVQPGGSLRLSCTTSGFTFSRYTMGWVRQAPGKGLEWVSAIGPPGSNTYYADSVKGRFTISRDNSKNTLYLQMNSLRAEDTAVYYCAKWVMLRGRFDYWGQGTLVTVSS

SEQ ID NO: 76 - protein, 123 aa, Artificial Sequence
  Description of sequence component of an antibody which targets the ASGR antigen
EVQLLESGGGLVQPGGSLRLSCAASGFTFEDYGMGWVRQAPGKGLEWVSAIGRNGSQTYYADSVKGRFTISRDNSKNTLYLQMNSLRAEDTAVYYCAKLRRGRGLNTFTLDYWGQGTLVTVSS

SEQ ID NO: 77 - protein, 123 aa, Artificial Sequence
  Description of sequence component of an antibody which targets the ASGR antigen
EVQLLESGGGLVQPGGSLRLSCAASGFTFGAAGMGWVRQAPGKGLEWVSAIGRNGSQTYYADSVKGRFTISRDNSKNTLYLQMNSLRAEDTAVYYCAKLRRGRGLNTFTLDYWGQGTLVTVSS

SEQ ID NO: 78 - protein, 108 aa, Artificial Sequence
  Description of sequence component of an antibody which targets the ASGR antigen
DIQMTQSPSSLSASVGDRVTITCRASQAIGRWLLWYQQKPGKAPKLLIGPGSRLRSGVPSRFSGSGSGTDFTLTISSLQPEDFVTYYCQQAYAWPPTFGQGTKVEIKR

SEQ ID NO: 79 - protein, 108 aa, Artificial Sequence
  Description of sequence component of an antibody which targets the ASGR antigen
DIQMTQSPSSLSASVGDRVTITCRASQAIGRWLLWYQQKPGKAPKHLIGPGSRLQSGVPSRFSGSGSGTDFTLTISSLQPEDFATYYCQQAYQLPVTFGQGTKVEIKR

SEQ ID NO: 80 - protein, 108 aa, Artificial Sequence
  Description of sequence component of an antibody which targets the ASGR antigen
DIQMTQSPSSLSASVGDRVTITCRASQAIGRWLLWYQQKPGKAPKLLIGPGSRLQSGVPSRFSGSGSGTDFTLTIGSLQPEDFATYYCQQAYSLPPTFGQGTKVEIKR

SEQ ID NO: 81 - protein, 108 aa, Artificial Sequence
  Description of sequence component of an antibody which targets the ASGR antigen
DIQMTQSPSSLSASVGDRVTITCRASGDIGHALWWYQQKPGKAPKLLIRGGSALQSGVPSRFSGSGSGTDFTLTISSLQPEDFATYYCGQSHVRPFTFGQGTKVEIKR

SEQ ID NO: 82 - protein, 108 aa, Artificial Sequence
  Description of sequence component of an antibody which targets the ASGR antigen
DIQMTQSPSSLSASVGDRVTITCQASKNIGERLVWYQQKPGKAPKLLIGFASLLQSGVPSRFSGSGSGTDFTLTISSLQPEDFATYYCGQYRWVPATFGQGTKVEIKR

SEQ ID NO: 83 - protein, 116 aa, Artificial Sequence
  Description of sequence component of an antibody which targets the ASGR antigen
EVQLLESGGGLVQPGGSLRLSCAASGFTFSTYPMHWARQAPGKGLEWVSSISPSGDSTYYADSVKGRFTISRDNSKNTLYLQMNSLRAEDTAVYYCAKNALRFDYWGQGTLVTVSS

SEQ ID NO: 84 - protein, 120 aa, Artificial Sequence
  Description of sequence component of an antibody which targets the ASGR antigen
EVQLLESGGGLVQPGGSLRLSCAASGFTFKPYAMHWVRQAPGKGLEWVSSISSTGLSTYYADSVKGRFTISRDNSKNTLYLQMNSLRAEDTAVYYCAKDASRFRQPFDYWGQGTLVTVSS

SEQ ID NO: 85 - protein, 121 aa, Artificial Sequence
  Description of sequence component of an antibody which targets the ASGR antigen
EVQLLESGGGLVQPGGSLRLSCAASGFTFPKYGMAWVRQAPGKGLEWVSRIGATGSETYYADSVKGRFTISRDNSKNTLYLQMNSLRAEDTAVYYCAKHRGTAHSSFFDYWGQGTLVTVSS

SEQ ID NO: 86 - protein, 120 aa, Artificial Sequence
  Description of sequence component of an antibody which targets the ASGR antigen
EVQLLESGGGLVQPGGSLRLSCAASGFTFSANGMHWVRQAPGKGLEWVSVISATGDQTYYADSVKGRFTISRDNSKNTLYLQMNSLRAEDTAVYYCAKGYDRRHRKFDYWGQGTLVTVSS

SEQ ID NO: 87 - protein, 122 aa, Artificial Sequence
  Description of sequence component of an antibody which targets the ASGR antigen
EVQLLESGGGLVQPGGSLRLSCAASGFTFADYSMYWVRQAPGKGLEWVSDISPSGSMTYYADSVKGRFTISRDNSKNTLYLQMNSLRAEDTAVYYCAKGLPGQNMHVGFDYWGQGTLVTVSS

SEQ ID NO: 88 - protein, 108 aa, Artificial Sequence
  Description of sequence component of an antibody which targets the ASGR antigen
DIQMTQSPSSLSASVGDRVTITCRASQAIGRWLLWYQQKPGKAPKLLIYAASRLQSGVPSRFSGSGSGTDFTLTISSLQPEDFATYYCQQAYSLPPTFGQGTKVEIKR

SEQ ID NO: 89 - protein, 108 aa, Artificial Sequence
  Description of sequence component of an antibody which targets the ASGR antigen
DIQMTQSPSSLSASVGDRVTITCRASMSIDESLWWYQQKPGKAPKLLIRGGSGLQSGVPSRFSGSGSGTDFTLTISSLQPEDFATYYCGQAARRPYTFGQGTKVEIKR

SEQ ID NO: 90 - protein, 108 aa, Artificial Sequence
  Description of sequence component of an antibody which targets the ASGR antigen
DIQMTQSPSSLSASVGDRVTITCRASHYIGNELWWYQQKPGKAPKLLIRRGSGLQSGVPSRFSGSGSGTDFTLTISSLQPEDFATYYCGQARHRPYTFGQGTRVEIKR

SEQ ID NO: 91 - protein, 108 aa, Artificial Sequence
  Description of sequence component of an antibody which targets the ASGR antigen
DIQMTQSPSSLSASVGDRVTITCRASSNIGRSLVWYQQKPGKAPKLLIAGGSLLQSGVPSRFSGSGSGTDFTLTISSLQPEDFATYYCGQYAEEPFTFGQGTRVEIKR

SEQ ID NO: 92 - protein, 108 aa, Artificial Sequence
  Description of sequence component of an antibody which targets the ASGR antigen
DIQMTQSPSSLSASVGDRVTITCRASVKIGERLWWYQQKPGEAPKLLIRDASLLQSGVPSRFSGSGSGTDFTLTISSLQPEDFATYYCGQSWMRPYTFGQGTKVEIKR

SEQ ID NO: 93 - protein, 108 aa, Artificial Sequence
  Description of sequence component of an antibody which targets the ASGR antigen
DIQMTQSPSSLSASVGDRVTITCRASSYIGGELWWYQQKPGKAPKLLISGTSGLQSGVPSRFSGSGSGTDFTLTISSLQPEDFATYYCGQAAKRPFTFGQGTKVEIKR

SEQ ID NO: 94 - protein, 108 aa, Artificial Sequence
  Description of sequence component of an antibody which targets the ASGR antigen
DIQMTQSPSSLSASVGDRVTITCRASSWINSDLVWYQQKPGEAPKLLIAGGSLLQSGVPSRFSGSGSGTDFTLTISSLQPEDFATYYCGQYLEEPYTFGQGTKVEVKR

SEQ ID NO: 95 - protein, 39 aa, Artificial Sequence
  Description of sequence hinge domain
IEVMYPPPYLDNEKSNGTIIHVKGKHLCPSPLFPGPSKP

SEQ ID NO: 96 - protein, 45 aa, Artificial Sequence
  Description of sequence hinge domain
TTTPAPRPPTPAPTIASQPLSLRPEACRPAAGGAVHTRGLDFACD

SEQ ID NO: 97 - protein, 10 aa, Artificial Sequence
  Description of sequence hinge domain
EQKLISEEDL

SEQ ID NO: 98 - protein, 43 aa, Artificial Sequence
  Description of sequence hinge domain
IEVEQKLISEEDLLDNEKSNGTIIHVKGKHLCPSPLFPGPSKP

SEQ ID NO: 99 - protein, 27 aa, Artificial Sequence
  Description of sequence transmembrane domain
FWVLVVVGGVLACYSLLVTVAFIIFWV

SEQ ID NO: 100 - protein, 66 aa, Artificial Sequence
  Description of sequence transmembrane domain
IEVMYPPPYLDNEKSNGTIIHVKGKHLCPSPLFPGPSKPFWVLVVVGGVLACYSLLVTVAFIIFWV

SEQ ID NO: 101 - protein, 70 aa, Artificial Sequence
  Description of sequence transmembrane domain
IEVEQKLISEEDLLDNEKSNGTIIHVKGKHLCPSPLFPGPSKPFWVLVVVGGVLACYSLLVTVAFIIFWV

SEQ ID NO: 102 - protein, 72 aa, Artificial Sequence
  Description of sequence transmembrane domain
TTTPAPRPPTPAPTIASQPLSLRPEACRPAAGGAVHTRGLDFACDFWVLVVVGGVLACYSLLVTVAFIIFWV

SEQ ID NO: 103 - protein, 112 aa, Artificial Sequence
  Description of sequence signalling domain
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 104 - protein, 41 aa, Artificial Sequence
  Description of sequence signalling domain
RSKRSRLLHSDYMNMTPRRPGPTRKHYQPYAPPRDFAAYRS

SEQ ID NO: 105 - protein, 48 aa, Artificial Sequence
  Description of sequence signalling domain
QRRKYRSNKGESPVEPAEPCHYSCPREEEGSTIPIQEDYRKPEPACSP

SEQ ID NO: 106 - protein, 42 aa, Artificial Sequence
  Description of sequence signalling domain
ALYLLRRDQRLPPDAHKPPGGGSFRTPIQEEQADAHSTLAKI

SEQ ID NO: 107 - protein, 42 aa, Artificial Sequence
  Description of sequence signalling domain
KRGRKKLLYIFKQPFMRPVQTTQEEDGCSCRFPEEEEGGCEL

SEQ ID NO: 108 - protein, 38 aa, Artificial Sequence
  Description of sequence signalling domain
CWLTKKKYSSSVHDPNGEYMFMRAVNTAKKSRLTDVTL

SEQ ID NO: 109 - protein, 197 aa, Artificial Sequence
  Description of sequence signalling domain
TYTYRHCWPHKPLVTADEAGMEALTPPPATHLSPLDSAHTLLAPPDSSEKICTVQLVGNSWTPGYPETQEALCPQVTWSWDQLPSRALGPAAAPTLSPESPAGSPAMMLQPGPQLYDVMDAVPARRWKEFVRTLGLREAEIEAVEVEIGRFRDQQYEMLKRWRQQQPAGLGAVYAALERMGLDGCVEDLRSRLQRGP

SEQ ID NO: 110 - protein, 153 aa, Artificial Sequence
  Description of sequence signalling domain
RSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 111 - protein, 286 aa, Artificial Sequence
  Description of sequence interleukin receptor peptide
NCRNTGPWLKKVLKCNTPDPSKFFSQLSSEHGGDVQKWLSSPFPSSSFSPGGLAPEISPLEVLERDKVTQLLLQQDKVPEPASLSSNHSLTSCFTNQGYFFFHLPDALEIEACQVYFTYDPYSEEDPDEGVAGAPTGSSPQPLQPLSGEDDAYCTFPSRDDLLLFSPSLLGGPSPPSTAPGGSGAGEERMPPSLQERVPRDWDPQPLGPPTPGVPDLVDFQPPPELVLREAGEEVPDAGPREGVSFPWSRPPGQGEFRALNARLPLNTDAYLSLQELQGQDPTHLV

SEQ ID NO: 112 - protein, 195 aa, Artificial Sequence
  Description of sequence interleukin receptor peptide
KKRIKPIVWPSLPDHKKTLEHLCKKPRKNLNVSFNPESFLDCQIHRVDDIQARDEVEGFLQDTFPQQLEESEKQRLGGDVQSPNCPSEDVVITPESFGRDSSLTCLAGNVSACDAPILSSSRSLDCRESGKNGPHVYQDLLLSLGTTNSTLPPPFSLQSGILTLNPVAQGQPILTSLGSNQEEAYVTMSSFYQNQ

SEQ ID NO: 113 - protein, 91 aa, Artificial Sequence
  Description of sequence interleukin receptor peptide
KKRIKPIVWPSLPDHKKTLEHLCKKPRKNLNVSFNPESFLDCQIHRVDDIQARDEVEGFLQDTFPQQPILTSLGSNQEEAYVTMSSFYQNQ

SEQ ID NO: 114 - protein, 230 aa, Artificial Sequence
  Description of sequence interleukin receptor peptide
KLSPRVKRIFYQNVPSPAMFFQPLYSVHNGNFQTWMGAHGAGVLLSQDCAGTPQGALEPCVQEATALLTCGPARPWKSVALEEEQEGPGTRLPGNLSSEDVLPAGCTEWRVQTLAYLPQEDWAPTSLTRPAPPDSEGSRSSSSSSSSNNNNYCALGCYGGWHLSALPGNTQSSGPIPALACGLSCDHQGLETQQGVAWVLAGHCQRPGLHEDLQGMLLPSVLSKARSWTF

SEQ ID NO: 115 - protein, 569 aa, Artificial Sequence
  Description of sequence interleukin receptor peptide
KIKKEWWDQIPNPARSRLVAIIIQDAQGSQWEKRSRGQEPAKCPHWKNCLTKLLPCFLEHNMKRDEDPHKAAKEMPFQGSGKSAWCPVEISKTVLWPESISVVRCVELFEAPVECEEEEEVEEEKGSFCASPESSRDDFQEGREGIVARLTESLFLDLLGEENGGFCQQDMGESCLLPPSGSTSAHMPWDEFPSAGPKEAPPWGKEQPLHLEPSPPASPTQSPDNLTCTETPLVIAGNPAYRSFSNSLSQSPCPRELGPDPLLARHLEEVEPEMPCVPQLSEPTTVPQPEPETWEQILRRNVLQHGAAAAPVSAPTSGYQEFVHAVEQGGTQASAVVGLGPPGEAGYKAFSSLLASSAVSPEKCGFGASSGEEGYKPFQDLIPGCPGDPAPVPVPLFTFGLDREPPRSPQSSHLPSSSPEHLGLEPGEKVEDMPKPPLPQEQATDPLVDSLGSGIVYSALTCHLCGHLKQCHGQEDGGQTPVMASPCCGCCCGDRSSPPTTPLRAPDPSPGGVPLEASLCPASLAPSGISEKSKSSSSFHPAPGNAQSSSQTPKIVNFVSVGPTYMRVS

SEQ ID NO: 116 - protein, 437 aa, Artificial Sequence
  Description of sequence interleukin receptor peptide
RFCGIYGYRLRRKWEEKIPNPSKSHLFQNGSAELWPPGSMSAFTSGSPPHQGPWGSRFPELEGVFPVGFGDSEVSPLTIEDPKHVCDPPSGPDTTPAASDLPTEQPPSPQPGPPAASHTPEKQASSFDFNGPYLGPPHSRSLPDILGQPEPPQEGGSQKSPPPGSLEYLCLPAGGQVQLVPLAQAMGPGQAVEVERRPSQGAAGSPSLESGGGPAPPALGPRVGGQDQKDSPVAIPMSSGDTEDPGVASGYVSSADLVFTPNSGASSVSLVPSLGLPSDQTPSLCPGLASGPPGAPGPVKSGFEGYVELPPIEGRSPRSPRNNPVPPEAKSPVLNPGERPADVSPTSPQPEGLLVLQQVGDYCFLPGLGPGPLSLRSKPSSPGPGPEIKNLDQAFQVKKPPGQAVPQVPVIQLFKALKQQDYLSLPPWEVNKPGEVC

SEQ ID NO: 117 - protein, 189 aa, Artificial Sequence
  Description of sequence interleukin receptor peptide
RHERIKKTSFSTTTLLPPIKVLVVYPSEICFHHTICYFTEFLQNHCRSEVILEKWQKKKIAEMGPVQWLATQKKAADKVVFLLSNDVNSVCDGTCGKSEGSPSENSQDLFPLAFNLFCSDLRSQIHLHKYVVVYFREIDTKDDYNALSVCPKYHLMKDATAFCAELLHVKQQVSAGKRSQACHDGCCSL

SEQ ID NO: 118 - protein, 4 aa, Artificial Sequence
  Description of sequence STAT5 association motif
  MISC_FEATURE (2)..(2): X is any amino acid
  MISC_FEATURE (3)..(3): any amino acid
  MISC_FEATURE (4)..(4): F or L
YXXX

SEQ ID NO: 119 - protein, 4 aa, Artificial Sequence
  Description of sequence STAT5 association motif
YCTF

SEQ ID NO: 120 - protein, 4 aa, Artificial Sequence
  Description of sequence STAT5 association motif
YFFF

SEQ ID NO: 121 - protein, 4 aa, Artificial Sequence
  Description of sequence STAT5 association motif
YLSL

SEQ ID NO: 122 - protein, 5 aa, Artificial Sequence
  Description of sequence STAT5 association motif
YLSLQ

SEQ ID NO: 123 - protein, 57 aa, Artificial Sequence
  Description of sequence JAK1- and/or a JAK2-binding motif
KVLKCNTPDPSKFFSQLSSEHGGDVQKWLSSPFPSSSFSPGGLAPEISPLEVLERDK

SEQ ID NO: 124 - protein, 45 aa, Artificial Sequence
  Description of sequence JAK1- and/or a JAK2-binding motif
NPWFQRAKMPRALDFSGHTHPVATFQPSRPESVNDLFLCPQKELT

SEQ ID NO: 125 - protein, 42 aa, Artificial Sequence
  Description of sequence JAK1- and/or a JAK2-binding motif
GYICLRNSLPKVLNFHNFLAWPFPNLPPLEAMDMVEVIYINR

SEQ ID NO: 126 - protein, 42 aa, Artificial Sequence
  Description of sequence JAK1- and/or a JAK2-binding motif
PLKEKSIILPKSLISVVRSATLETKPESKYVSLITSYQPFSL

SEQ ID NO: 127 - protein, 43 aa, Artificial Sequence
  Description of sequence JAK1- and/or a JAK2-binding motif
RRRKKLPSVLLFKKPSPFIFISQRPSPETQDTIHPLDEEAFLK

SEQ ID NO: 128 - protein, 45 aa, Artificial Sequence
  Description of sequence JAK1- and/or a JAK2-binding motif
YIHVGKEKHPANLILIYGNEFDKRFFVPAEKIVINFITLNISDDS

SEQ ID NO: 129 - protein, 41 aa, Artificial Sequence
  Description of sequence JAK1- and/or a JAK2-binding motif
RYVTKPPAPPNSLNVQRVLTFQPLRFIQEHVLIPVFDLSGP

SEQ ID NO: 130 - protein, 43 aa, Artificial Sequence
  Description of sequence JAK1- and/or a JAK2-binding motif
NYVFFPSLKPSSSIDEYFSEQPLKNLLLSTSEEQIEKCFIIEN

SEQ ID NO: 131 - protein, 49 aa, Artificial Sequence
  Description of sequence JAK1- and/or a JAK2-binding motif
YWFHTPPSIPLQIEEYLKDPTQPILEALDKDSSPKDDVWDSVSIISFPE

SEQ ID NO: 132 - protein, 51 aa, Artificial Sequence
  Description of sequence JAK1- and/or a JAK2-binding motif
YAFSPRNSLPQHLKEFLGHPHHNTLLFFSFPLSDENDVFDKLSVIAEDSES

SEQ ID NO: 133 - protein, 4 aa, Artificial Sequence
  Description of sequenceSTAT3 association motif
  MISC_FEATURE (2)..(2): any amino acid
  MISC_FEATURE (2)..(2): X is any amino acid
  MISC_FEATURE (3)..(3): any amino acid
YXXQ

SEQ ID NO: 134 - protein, 286 aa, Artificial Sequence; identical to SEQ ID NO: 111
  Description of sequenceII_2Rb endodomain portion

SEQ ID NO: 135 - protein, 94 aa, Artificial Sequence
  Description of sequenceII_2Rb endodomain portion
NCRNTGPWLKKVLKCNTPDPSKFFSQLSSEHGGDVQKWLSSPFPSSSFSPGGLAPEISPLEVLERDKVTQLLPLNTDAYLSLQELQGQDPTHLV

SEQ ID NO: 136 - protein, 208 aa, Artificial Sequence
  Description of sequenceII_2Rb endodomain portion
NCRNTGPWLKKVLKCNTPDPSKFFSQLSSEHGGDVQKWLSSPFPSSSFSPGGLAPEISPLEVLERDKVTQLLDAYCTFPSRDDLLLFSPSLLGGPSPPSTAPGGSGAGEERMPPSLQERVPRDWDPQPLGPPTPGVPDLVDFQPPPELVLREAGEEVPDAGPREGVSFPWSRPPGQGEFRALNARLPLNTDAYLSLQELQGQDPTHLV

SEQ ID NO: 137 - protein, 52 aa, Artificial Sequence
  Description of sequenceJAK3-binding motif
ERTMPRIPTLKNLEDLVTEYHGNFSAWSGVSKGLAESLQPDYSERLCLVSEI

SEQ ID NO: 138 - protein, 86 aa, Artificial Sequence
  Description of sequenceJAK3-binding motif
ERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 139 - protein, 299 aa, Artificial Sequence
  Description of sequenceillustrative domain sequence
RSKRSRLLHSDYMNMTPRRPGPTRKHYQPYAPPRDFAAYRSERTMPRIPTLKNLEDLVTEYHGNFSAWSGVSKGLAESLQPDYSERLCLVSEINCRNTGPWLKKVLKCNTPDPSKFFSQLSSEHGGDVQKWLSSPFPSSSFSPGGLAPEISPLEVLERDKVTQLLPLNTDAYLSLQELQGQDPTHLVRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 140 - protein, 317 aa, Artificial Sequence
  Description of sequenceillustrative domain sequence
RSKRSRLLHSDYMNMTPRRPGPTRKHYQPYAPPRDFAAYRSERTMPRIPTLKNLEDLVTEYHGNFSAWSGVSKGLAESLQPDYSERLCLVSEIKKRIKPIVWPSLPDHKKTLEHLCKKPRKNLNVSFNPESFLDCQIHRVDDIQARDEVEGFLQDTFPQQPILTSLGSNQEEAYVTMSSFYQNQRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPG

SEQ ID NO: 141 - protein, 21 aa, Artificial Sequence
  Description of sequenceillustrative domain sequence
MALPVTALLLPLALLLHAARP

SEQ ID NO: 142 - protein, 239 aa, Artificial Sequence
  Description of sequenceillustrative domain sequence
MVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSKLSKDPNEKRDHMVLLEFVTAAGITLGMDELYK

SEQ ID NO: 143 - protein, 22 aa, Artificial Sequence
  Description of sequenceCleavage domain sequence
GSGATNFSLLKQAGDVEENPGP

SEQ ID NO: 144 - protein, 21 aa, Artificial Sequence
  Description of sequenceCleavage domain sequence
GSGEGRGSLLTCGDVEENPGP

SEQ ID NO: 145 - protein, 23 aa, Artificial Sequence
  Description of sequenceCleavage domain sequence
GSGQCTNYALLKLAGDVESNPGP

SEQ ID NO: 146 - protein, 25 aa, Artificial Sequence
  Description of sequenceCleavage domain sequence
GSGVKQTLNFDLLKLAGDVESNPGP

SEQ ID NO: 147 - protein, 4 aa, Artificial Sequence
  Description of sequenceCleavage domain sequence
  MISC_FEATURE (2)..(2): any amino acid
  MISC_FEATURE (2)..(2): X is any amino acid
  MISC_FEATURE (3)..(3): any amino acid
RXXR

SEQ ID NO: 148 - protein, 4 aa, Artificial Sequence
  Description of sequenceCleavage domain sequence
RRKR

SEQ ID NO: 149 - protein, 366 aa, Artificial Sequence
  Description of sequenceillustrative CAR Sequence
MALPVTALLLPLALLLHAARPEVQLLESGGGLVQPGGSLRLSCAASGFTFEKYAMAWVRQAPGKGLEWVSRISARGVTTYYADSVKGRFTISRDNSKNTLYLQMNSLRAEDTAVYYCAKHKRHEHTRFDSWGQGTLVTVSSTTTPAPRPPTPAPTIASQPLSLRPEACRPAAGGAVHTRGLDFACDFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 150 - protein, 367 aa, Artificial Sequence
  Description of sequenceillustrative CAR Sequence
MALPVTALLLPLALLLHAARPEVQLLESGGGLVQPGGSLRLSCAASGFTFEKYAMAWVRQAPGKGLEWVSRISARGVTTYYADSVKGRFTISRDNSKNTLYLQMNSLRAEDTAVYYCAKHKRHEHTRFDSWGQGTLVTVSSAAAIEVEQKLISEEDLLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 151 - protein, 638 aa, Artificial Sequence
  Description of sequenceillustrative CAR Sequence
MALPVTALLLPLALLLHAARPEVQLLESGGGLVQPGGSLRLSCAASGFTFEKYAMAWVRQAPGKGLEWVSRISARGVTTYYADSVKGRFTISRDNSKNTLYLQMNSLRAEDTAVYYCAKHKRHEHTRFDSWGQGTLVTVSSTTTPAPRPPTPAPTIASQPLSLRPEACRPAAGGAVHTRGLDFACDFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRRRKRGSGATNFSLLKQAGDVEENPGPTRGGGATMVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSKLSKDPNEKRDHMVLLEFVTAAGITLGMDELYK

SEQ ID NO: 152 - protein, 639 aa, Artificial Sequence
  Description of sequenceillustrative CAR Sequence
MALPVTALLLPLALLLHAARPEVQLLESGGGLVQPGGSLRLSCAASGFTFEKYAMAWVRQAPGKGLEWVSRISARGVTTYYADSVKGRFTISRDNSKNTLYLQMNSLRAEDTAVYYCAKHKRHEHTRFDSWGQGTLVTVSSAAAIEVEQKLISEEDLLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRRRKRGSGATNFSLLKQAGDVEENPGPTRGGGATMVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSKLSKDPNEKRDHMVLLEFVTAAGITLGMDELYK

SEQ ID NO: 153 - DNA, 1101 bp, Artificial Sequence
  Description of sequenceillustrative CAR sequence
atggccttaccagtgaccgccttgctcctgccgctggccttgctgctccacgccgccaggccggaggtgcagctgttggagtctgggggaggcttggtacagcctggggggtccctgcgtctctcctgtgcagcctccggattcacctttgagaagtatgcgatggcgtgggtccgccaggccccagggaagggtctggagtgggtctcacggatttcggcgaggggtgtgacgacatactacgcagactccgtgaagggccggttcaccatctcccgcgacaattccaagaacacgctgtatctgcaaatgaacagcctgcgtgctgaggacaccgcggtatattactgtgcgaaacataagcggcacgagcatactcgttttgactcctggggtcagggaaccctggtcaccgtctcgagcaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatttctgggtgctggtggtggtgggcggcgtgctggcctgctacagcctgctggtgaccgtggccttcatcatcttctgggtgcggagcaagcggagccggctgctgcacagcgactacatgaacatgaccccccggcggcctgggcccacccgcaagcattaccagccctatgccccaccacgcgacttcgcagcctatcgctccagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaa

SEQ ID NO: 154 - DNA, 1104 bp, Artificial Sequence
  Description of sequenceillustrative CAR sequence
atggccttaccagtgaccgccttgctcctgccgctggccttgctgctccacgccgccaggccggaggtgcagctgttggagtctgggggaggcttggtacagcctggggggtccctgcgtctctcctgtgcagcctccggattcacctttgagaagtatgcgatggcgtgggtccgccaggccccagggaagggtctggagtgggtctcacggatttcggcgaggggtgtgacgacatactacgcagactccgtgaagggccggttcaccatctcccgcgacaattccaagaacacgctgtatctgcaaatgaacagcctgcgtgctgaggacaccgcggtatattactgtgcgaaacataagcggcacgagcatactcgttttgactcctggggtcagggaaccctggtcaccgtctcgagcgcggccgccatcgaggtggagcagaagctgatcagcgaggaggacctgctggacaacgagaagagcaacggcaccatcatccacgtgaagggcaagcacctgtgccccagccccctgttccccggccccagcaagcccttctgggtgctggtggtggtgggcggcgtgctggcctgctacagcctgctggtgaccgtggccttcatcatcttctgggtgcggagcaagcggagccggctgctgcacagcgactacatgaacatgaccccccggcggcctgggcccacccgcaagcattaccagccctatgccccaccacgcgacttcgcagcctatcgctccagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaa

SEQ ID NO: 155 - DNA, 1917 bp, Artificial Sequence
  Description of sequenceillustrative CAR sequence
atggccttaccagtgaccgccttgctcctgccgctggccttgctgctccacgccgccaggccggaggtgcagctgttggagtctgggggaggcttggtacagcctggggggtccctgcgtctctcctgtgcagcctccggattcacctttgagaagtatgcgatggcgtgggtccgccaggccccagggaagggtctggagtgggtctcacggatttcggcgaggggtgtgacgacatactacgcagactccgtgaagggccggttcaccatctcccgcgacaattccaagaacacgctgtatctgcaaatgaacagcctgcgtgctgaggacaccgcggtatattactgtgcgaaacataagcggcacgagcatactcgttttgactcctggggtcagggaaccctggtcaccgtctcgagcaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatttctgggtgctggtggtggtgggcggcgtgctggcctgctacagcctgctggtgaccgtggccttcatcatcttctgggtgcggagcaagcggagccggctgctgcacagcgactacatgaacatgaccccccggcggcctgggcccacccgcaagcattaccagccctatgccccaccacgcgacttcgcagcctatcgctccagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcaggagaaaaagaggaagcggagctactaacttcagcctgctgaagcaggctggagacgtggaggagaaccctggacctacgcgtggaggtggcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccaagctgagcaaagaccccaacgagaagcgcgatcacatggtcctgctggagttcgtgaccgccgccgggatcactctcggcatggacgagctgtacaagtaa

SEQ ID NO: 156 - DNA, 1920 bp, Artificial Sequence
  Description of sequenceillustrative CAR sequence
atggccttaccagtgaccgccttgctcctgccgctggccttgctgctccacgccgccaggccggaggtgcagctgttggagtctgggggaggcttggtacagcctggggggtccctgcgtctctcctgtgcagcctccggattcacctttgagaagtatgcgatggcgtgggtccgccaggccccagggaagggtctggagtgggtctcacggatttcggcgaggggtgtgacgacatactacgcagactccgtgaagggccggttcaccatctcccgcgacaattccaagaacacgctgtatctgcaaatgaacagcctgcgtgctgaggacaccgcggtatattactgtgcgaaacataagcggcacgagcatactcgttttgactcctggggtcagggaaccctggtcaccgtctcgagcgcggccgccatcgaggtggagcagaagctgatcagcgaggaggacctgctggacaacgagaagagcaacggcaccatcatccacgtgaagggcaagcacctgtgccccagccccctgttccccggccccagcaagcccttctgggtgctggtggtggtgggcggcgtgctggcctgctacagcctgctggtgaccgtggccttcatcatcttctgggtgcggagcaagcggagccggctgctgcacagcgactacatgaacatgaccccccggcggcctgggcccacccgcaagcattaccagccctatgccccaccacgcgacttcgcagcctatcgctccagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcaggagaaaaagaggaagcggagctactaacttcagcctgctgaagcaggctggagacgtggaggagaaccctggacctacgcgtggaggtggcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccaagctgagcaaagaccccaacgagaagcgcgatcacatggtcctgctggagttcgtgaccgccgccgggatcactctcggcatggacgagctgtacaagtaa

SEQ ID NO: 157 - protein, 431 aa, Homo sapiens
MPNPRPGKPSAPSLALGPSPGASPSWRAAPKASDLLGARGPGGTFQGRDLRGGAHASSSSLNPMPPSQLQLPTLPLVMVAPSGARLGPLPHLQALLQDRPHFMHQLSTVDAHARTPVLQVHPLESPAMISLTPPTTATGVFSLKARPGLPPGINVASLEWVSREPALLCTFPNPSAPRKDSTLSAVPQSSYPLLANGVCKWPGCEKVFEEPEDFLKHCQADHLLDEKGRAQCLLQREMVQSLEQQLVLEKEKLSAMQAHLAGKMALTKASSVASSDKGSCCIVAAGSQGPVVPAWSGPREAPDSLFAVRRHLWGSHGNSTFPEFLHNMDYFKFHNMRPPFTYATLIRWAILEAPEKQRTLNEIYHWFTRMFAFFRNHPATWKNAIRHNLSLHKCFVRVESEKGAVWTVDELEFRKKRSQRPSRCSNPTPGP

SEQ ID NO: 158 - protein, 431 aa, Artificial Sequence
  Description of sequence component of an antibody which targets the ASGR antigen
MPNPRPGKPSAPSLALGPSPGASPSWRAAPKASDLLGARGPGGTFQGRDLRGGAHASSSSLNPMPPSQLQLPTLPLVMVAPSGARLGPLPHLQALLQDRPHFMHQLSTVDAHARTPVLQVHPLESPAMISLTPPTTATGVFSLKARPGLPPGINVASLEWVSREPALLCTFPNPSAPRKDSTLSAVPQSSYPLLANGVCKWPGCEKVFEEPEDFLKHCQADHLLDEKGRAQCLLQREMVQSLEQQLVLEKEKLSAMQAHLAGKMALTKASSVASSDKGSCCIVAAGSQGPVVPAWSGPREAPDSLFAVRRHLWGSHGNSTFPEFLHNMDYFKFHNMRPPFTYATLIRWAILEAPEKQRTLNEIYHWFTRMFAFFRNHPATWKNAIRHNLSLHKCFVRVESEKGAVWTVDELEFRKKREQRPSRCSNPTPGP

SEQ ID NO: 159 - protein, 431 aa, Artificial Sequence
  Description of sequence component of an antibody which targets the ASGR antigen
MPNPRPGKPSAPSLALGPSPGASPSWRAAPKASDLLGARGPGGTFQGRDLRGGAHASSSSLNPMPPSQLQLPTLPLVMVAPSGARLGPLPHLQALLQDRPHFMHQLSTVDAHARTPVLQVHPLESPAMISLTPPTTATGVFSLKARPGLPPGINVASLEWVSREPALLCTFPNPSAPRKDSTLSAVPQSSYPLLANGVCKWPGCEKVFEEPEDFLKHCQADHLLDEKGRAQCLLQREMVQSLEQQLVLEKEKLSAMQAHLAGKMALTKASSVASSDKGSCCIVAAGSQGPVVPAWSGPREAPDSLFAVRRHLWGSHGNSTFPEFLHNMDYFKFHNMRPPFTYATLIRWAILEAPEKQRTLNEIYHWFTRMFAFFRNHPATWKNAIRHNLSLHKCFVRVESEKGAVWTVDELEFRKKRSQRPARCSNPTPGP

SEQ ID NO: 160 - protein, 431 aa, Artificial Sequence
  Description of sequence component of an antibody which targets the ASGR antigen
MPNPRPGKPSAPSLALGPSPGASPSWRAAPKASDLLGARGPGGTFQGRDLRGGAHASSSSLNPMPPSQLQLPTLPLVMVAPSGARLGPLPHLQALLQDRPHFMHQLSTVDAHARTPVLQVHPLESPAMISLTPPTTATGVFSLKARPGLPPGINVASLEWVSREPALLCTFPNPSAPRKDSTLSAVPQSSYPLLANGVCKWPGCEKVFEEPEDFLKHCQADHLLDEKGRAQCLLQREMVQSLEQQLVLEKEKLSAMQAHLAGKMALTKASSVASSDKGSCCIVAAGSQGPVVPAWSGPREAPDSLFAVRRHLWGSHGNSTFPEFLHNMDYFKFHNMRPPFTYATLIRWAILEAPEKQRTLNEIYHWFTRMFAFFRNHPATWKNAIRHNLSLHKCFVRVESEKGAVWTVDELEFRKKREQRPARCSNPTPGP

SEQ ID NO: 161 - protein, 362 aa, Artificial Sequence
  Description of sequence component of an antibody which targets the ASGR antigen
GGAHASSSSLNPMPPSQLQLPTLPLVMVAPSGARLGPLPHLQALLQDRPHFMHQLSTVDAHARTPVLQVHPLESPAMISLTPPTTATGVFSLKARPGLPPGINVASLEWVSREPALLCTFPNPSAPRKDSTLSAVPQSSYPLLANGVCKWPGCEKVFEEPEDFLKHCQADHLLDEKGRAQCLLQREMVQSLEQQLVLEKEKLSAMQAHLAGKMALTKASSVASSDKGSCCIVAAGSQGPVVPAWSGPREAPDSLFAVRRHLWGSHGNSTFPEFLHNMDYFKFHNMRPPFTYATLIRWAILEAPEKQRTLNEIYHWFTRMFAFFRNHPATWKNAIRHNLSLHKCFVRVESEKGAVWTVDELEF

SEQ ID NO: 162 - protein, 441 aa, Artificial Sequence
  Description of sequence component of an antibody which targets the ASGR antigen
MPNPRPGKPSAPSLALGPSPGASPSWRAAPKASDLLGARGPGGTFQGRDLRGGAHASSSSLNPMPPSQLQLPTLPLVMVAPSGARLGPLPHLQALLQDRPHFMHQLSTVDAHARTPVLQVHPLESPAMISLTPPTTATGVFSLKARPGLPPGINVASLEWVSREPALLCTFPNPSAPRKDSTLSAVPQSSYPLLANGVCKWPGCEKVFEEPEDFLKHCQADHLLDEKGRAQCLLQREMVQSLEQVEELSAMQAHLAGKMALTKASSVASSDKGSCCIVAAGSQGPVVPAWSGPREAPDSLFAVRRHLWGSHGNSTFPEFLHNMDYFKFHNMRPPFTYATLIRWAILEAPEKQRTLNEIYHWFTRMFAFFRNHPATWKNAIRHNLSLHKCFVRVESEKGAVWTVDELEFRKKRSQRPSRCSNPTPGPEGRGSLLTCGDVEEN

SEQ ID NO: 163 - DNA, 1296 bp, Artificial Sequence
  Description of sequence component of an antibody which targets the ASGR antigen
atgcccaaccccaggcctggcaagccctcggccccttccttggcccttggcccatccccaggagcctcgcccagctggagggctgcacccaaagcctcagacctgctgggggcccggggcccagggggaaccttccagggccgagatcttcgaggcggggcccatgcctcctcttcttccttgaaccccatgccaccatcgcagctgcagctgcccacactgcccctagtcatggtggcaccctccggggcacggctgggccccttgccccacttacaggcactcctccaggacaggccacatttcatgcaccagctctcaacggtggatgcccacgcccggacccctgtgctgcaggtgcaccccctggagagcccagccatgatcagcctcacaccacccaccaccgccactggggtcttctccctcaaggcccggcctggcctcccacctgggatcaacgtggccagcctggaatgggtgtccagggagccggcactgctctgcaccttcccaaatcccagtgcacccaggaaggacagcaccctttcggctgtgccccagagctcctacccactgctggcaaatggtgtctgcaagtggcccggatgtgagaaggtcttcgaagagccagaggacttcctcaagcactgccaggcggaccatcttctggatgagaagggcagggcacaatgtctcctccagagagagatggtacagtctctggagcagcagctggtgctggagaaggagaagctgagtgccatgcaggcccacctggctgggaaaatggcactgaccaaggcttcatctgtggcatcatccgacaagggctcctgctgcatcgtagctgctggcagccaaggccctgtcgtcccagcctggtctggcccccgggaggcccctgacagcctgtttgctgtccggaggcacctgtggggtagccatggaaacagcacattcccagagttcctccacaacatggactacttcaagttccacaacatgcgaccccctttcacctacgccacgctcatccgctgggccatcctggaggctccagagaagcagcggacactcaatgagatctaccactggttcacacgcatgtttgccttcttcagaaaccatcctgccacctggaagaacgccatccgccacaacctgagtctgcacaagtgctttgtgcgggtggagagcgagaagggggctgtgtggaccgtggatgagctggagttccgcaagaaacggagccagaggcccagcaggtgttccaaccctacacctggcccctga

SEQ ID NO: 164 - DNA, 1352 bp, Artificial Sequence
  Description of sequence component of an antibody which targets the ASGR antigen
gaattcgtcgacatgcccaaccccagacccggcaagccttctgccccttctctggccctgggaccatctcctggcgcctccccatcttggagagccgcccctaaagccagcgatctgctgggagctagaggccctggcggcacattccagggcagagatctgagaggcggagcccacgcctctagcagcagcctgaatcccatgccccctagccagctgcagctgcctacactgcctctcgtgatggtggcccctagcggagctagactgggccctctgcctcatctgcaggctctgctgcaggaccggccccactttatgcaccagctgagcaccgtggacgcccacgccagaacacctgtgctgcaggtgcaccccctggaaagccctgccatgatcagcctgacccctccaaccacagccaccggcgtgttcagcctgaaggccagacctggactgccccctggcatcaatgtggccagcctggaatgggtgtcccgcgaacctgccctgctgtgcaccttccccaatcctagcgcccccagaaaggacagcacactgtctgccgtgccccagagcagctatcccctgctggctaacggcgtgtgcaagtggcctggctgcgagaaggtgttcgaggaacccgaggacttcctgaagcactgccaggccgaccatctgctggacgagaaaggcagagcccagtgcctgctgcagcgcgagatggtgcagtccctggaacagcagctggtgctggaaaaagaaaagctgagcgccatgcaggcccacctggccggaaagatggccctgacaaaagccagcagcgtggccagctccgacaagggcagctgttgtatcgtggccgctggcagccagggacctgtggtgcctgcttggagcggacctagagaggcccccgatagcctgtttgccgtgcggagacacctgtggggcagccacggcaactctaccttccccgagttcctgcacaacatggactacttcaagttccacaacatgaggccccccttcacctacgccaccctgatcagatgggccattctggaagcccccgagaagcagcggaccctgaacgagatctaccactggtttacccggatgttcgccttcttccggaaccaccccgccacctggaagaacgccatccggcacaatctgagcctgcacaagtgcttcgtgcgggtggaaagcgagaagggcgccgtgtggacagtggacgagctggaatttcggaagaagcggtcccagaggcccagccggtgtagcaatcctacacctggccctgagggcagaggaagtctgctaacatgcggtgacgtcgaggagaatcc

SEQ ID NO: 165 - protein, 21 aa, Artificial Sequence
  Description of sequence component of an antibody which targets the ASGR antigen
IYIWAPLAGTCGVLLLSLVIT

SEQ ID NO: 166 - protein, 60 aa, Artificial Sequence
  Description of sequence transmembrane domain
IEVMYPPPYLDNEKSNGTIIHVKGKHLCPSPLFPGPSKPIYIWAPLAGTCGVLLLSLVIT

SEQ ID NO: 167 - protein, 496 aa, Artificial Sequence
  Description of sequenceillustrative CAR sequence
MALPVTALLLPLALLLHAARPEVQLLESGGGLVQPGGSLRLSCAASGFTFEKYAMAWVRQAPGKGLEWVSRISARGVTTYYADSVKGRFTISRDNSKNTLYLQMNSLRAEDTAVYYCAKHKRHEHTRFDSWGQGTLVTVSSLVTVSSGGGGSGGGGSGGGGSDIQMTQSPSSLSASVGDRVTITCRASQAIGRWLLWYQQKPGKAPKLLIGPGSRLRSGVPSRFSGSGSGTDFTLTISSLQPEDFVTYYCQQAYAWPPTFGQGTKVEIKRAAAIEVEQKLISEEDLLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 168 - protein, 495 aa, Artificial Sequence
  Description of sequenceillustrative CAR sequence
MALPVTALLLPLALLLHAARPELQLLEFGGGLVQPGGSLRLSCTTSGFTFSRYTMGWVRQAPGKGLEWVSAIGPPGSNTYYADSVKGRFTISRDNSKNTLYLQMNSLRAEDTAVYYCAKWVMLRGRFDYWGQGTLVTVSSLVTVSSGGGGSGGGGSGGGGSDIQMTQSPSSLSASVGDRVTITCRASQAIGRWLLWYQQKPGKAPKHLIGPGSRLQSGVPSRFSGSGSGTDFTLTISSLQPEDFATYYCQQAYQLPVTFGQGTKVEIKRAAAIEVEQKLISEEDLLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 169 - protein, 499 aa, Artificial Sequence
  Description of sequenceillustrative CAR sequence
MALPVTALLLPLALLLHAARPEVQLLESGGGLVQPGGSLRLSCAASGFTFEDYGMGWVRQAPGKGLEWVSAIGRNGSQTYYADSVKGRFTISRDNSKNTLYLQMNSLRAEDTAVYYCAKLRRGRGLNTFTLDYWGQGTLVTVSSLVTVSSGGGGSGGGGSGGGGSDIQMTQSPSSLSASVGDRVTITCRASQAIGRWLLWYQQKPGKAPKLLIGPGSRLQSGVPSRFSGSGSGTDFTLTIGSLQPEDFATYYCQQAYSLPPTFGQGTKVEIKRAAAIEVEQKLISEEDLLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 170 - DNA, 1488 bp, Artificial Sequence
  Description of sequenceillustrative CAR sequence
atggccttaccagtgaccgccttgctcctgccgctggccttgctgctccacgccgccaggccggaggtgcagctgttggagtctgggggaggcttggtacagcctggggggtccctgcgtctctcctgtgcagcctccggattcacctttgagaagtatgcgatggcgtgggtccgccaggccccagggaagggtctggagtgggtctcacggatttcggcgaggggtgtgacgacatactacgcagactccgtgaagggccggttcaccatctcccgcgacaattccaagaacacgctgtatctgcaaatgaacagcctgcgtgctgaggacaccgcggtatattactgtgcgaaacataagcggcacgagcatactcgttttgactcctggggtcagggaaccctggtcaccgtctcgagcctggtgaccgtgagcagcggcggcggaggcagcggtggcggaggcagcggcggaggcggtagcgacatccagatgacccagtctccatcctccctgtctgcatctgtaggagaccgtgtcaccattacttgccgggcaagtcaggcgattgggcggtggttattgtggtatcagcagaaaccagggaaagcccctaagctcctgatcggtccgggttcccggttgcgaagtggggtcccatcacgtttcagtggcagtggatctgggacagatttcactctcaccatcagcagtctacaacctgaagattttgttacgtactactgtcaacaggcgtatgcctggcctccgacgttcggccaagggaccaaggtggaaatcaaacgggcggccgccatcgaggtggagcagaagctgatcagcgaggaggacctgctggacaacgagaagagcaacggcaccatcatccacgtgaagggcaagcacctgtgccccagccccctgttccccggccccagcaagcccttctgggtgctggtggtggtgggcggcgtgctggcctgctacagcctgctggtgaccgtggccttcatcatcttctgggtgcggagcaagcggagccggctgctgcacagcgactacatgaacatgaccccccggcggcctgggcccacccgcaagcattaccagccctatgccccaccacgcgacttcgcagcctatcgctccagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 171 - DNA, 1485 bp, Artificial Sequence
  Description of sequenceillustrative CAR sequence
atggccttaccagtgaccgccttgctcctgccgctggccttgctgctccacgccgccaggccggagttgcagctgttggagtttgggggaggcttggtacagcctggggggtccctgcgtctctcctgtacaacctccggattcaccttttcgaggtatactatgggttgggtccgccaggctccagggaagggtctagagtgggtctcagccattggcccgcccgggtcgaacacatactacgcagactccgtgaagggtcggttcaccatctcccgcgacaattccaagaacacgctgtatctgcaaatgaacagcctgcgtgccgaggacaccgcggtatattactgtgcgaaatgggtgatgttgcgggggcgctttgactactggggtcagggaaccctggtcaccgtctcgagcctggtgaccgtgagcagcggcggcggaggcagcggtggcggaggcagcggcggaggcggtagcgacatccagatgacccagtccccatcctccctgtctgcatctgtaggagaccgtgtcaccattacttgccgggcaagtcaggcgattgggcggtggttattgtggtatcagcagaaaccagggaaagcccctaagcacctgatcggtccgggttcccggttgcaaagtggggtcccatcacgtttcagtggcagtggatctgggacagatttcactctcaccatcagcagtctgcaacctgaagattttgctacgtactactgtcaacaggcgtatcagctgcctgtcacgttcggccaagggaccaaggtggaaatcaaacgggcggccgccatcgaggtggagcagaagctgatcagcgaggaggacctgctggacaacgagaagagcaacggcaccatcatccacgtgaagggcaagcacctgtgccccagccccctgttccccggccccagcaagcccttctgggtgctggtggtggtgggcggcgtgctggcctgctacagcctgctggtgaccgtggccttcatcatcttctgggtgcggagcaagcggagccggctgctgcacagcgactacatgaacatgaccccccggcggcctgggcccacccgcaagcattaccagccctatgccccaccacgcgacttcgcagcctatcgctccagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 172 - DNA, 1497 bp, Artificial Sequence
  Description of sequenceillustrative CAR sequence
atggccttaccagtgaccgccttgctcctgccgctggccttgctgctccacgccgccaggccggaggtgcagctgttggagtctgggggaggcttggtacagcctggggggtccctgcgtctctcctgtgcagcctccggattcacctttgaggattatggtatggggtgggtccgccaggctccaggaaagggtctagagtgggtctcagcgattggccgcaacgggtcgcagacatactacgcagactccgtgaagggccggttcaccatctcccgcgacaattccaagaacacgctgtatctgcaaatgaacagcctgcgtgccgaggataccgcggtatattactgtgcgaaacttcggagggggcggggtctgaatacgtttacgttagactactggggtcaaggaaccctggtcaccgtctcaagcctggtgaccgtgagcagcggcggcggaggcagcggtggcggaggcagcggcggaggcggtagcgacatccagatgacccagtccccatcctccctgtctgcatctgtaggagaccgtgtcaccattacttgccgggcaagtcaggcgattgggcggtggttattgtggtatcagcagaaaccagggaaagcccctaagctcctgatcggtccgggttcccggttgcaaagtggggtcccatcacgtttcagtggcagtggatctgggacagatttcactctcaccatcggcagtctgcaacctgaagattttgctacgtactactgtcaacaggcgtatagtctgcctccgacgttcggccaagggaccaaggtggaaatcaaacgggcggccgccatcgaggtggagcagaagctgatcagcgaggaggacctgctggacaacgagaagagcaacggcaccatcatccacgtgaagggcaagcacctgtgccccagccccctgttccccggccccagcaagcccttctgggtgctggtggtggtgggcggcgtgctggcctgctacagcctgctggtgaccgtggccttcatcatcttctgggtgcggagcaagcggagccggctgctgcacagcgactacatgaacatgaccccccggcggcctgggcccacccgcaagcattaccagccctatgccccaccacgcgacttcgcagcctatcgctccagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 173 - protein, 249 aa, Artificial Sequence
  Description of sequence scFv fragment
EVQLLESGGGLVQPGGSLRLSCAASGFTFEKYAMAWVRQAPGKGLEWVSRISARGVTTYYADSVKGRFTISRDNSKNTLYLQMNSLRAEDTAVYYCAKHKRHEHTRFDSWGQGTLVTVSSLVTVSSGGGGSGGGGSGGGGSDIQMTQSPSSLSASVGDRVTITCRASQAIGRWLLWYQQKPGKAPKLLIGPGSRLRSGVPSRFSGSGSGTDFTLTISSLQPEDFVTYYCQQAYAWPPTFGQGTKVEIKR

SEQ ID NO: 174 - protein, 248 aa, Artificial Sequence
  Description of sequence scFv fragment
ELQLLEFGGGLVQPGGSLRLSCTTSGFTFSRYTMGWVRQAPGKGLEWVSAIGPPGSNTYYADSVKGRFTISRDNSKNTLYLQMNSLRAEDTAVYYCAKWVMLRGRFDYWGQGTLVTVSSLVTVSSGGGGSGGGGSGGGGSDIQMTQSPSSLSASVGDRVTITCRASQAIGRWLLWYQQKPGKAPKHLIGPGSRLQSGVPSRFSGSGSGTDFTLTISSLQPEDFATYYCQQAYQLPVTFGQGTKVEIKR

SEQ ID NO: 175 - protein, 252 aa, Artificial Sequence
  Description of sequence scFv fragment
EVQLLESGGGLVQPGGSLRLSCAASGFTFEDYGMGWVRQAPGKGLEWVSAIGRNGSQTYYADSVKGRFTISRDNSKNTLYLQMNSLRAEDTAVYYCAKLRRGRGLNTFTLDYWGQGTLVTVSSLVTVSSGGGGSGGGGSGGGGSDIQMTQSPSSLSASVGDRVTITCRASQAIGRWLLWYQQKPGKAPKLLIGPGSRLQSGVPSRFSGSGSGTDFTLTIGSLQPEDFATYYCQQAYSLPPTFGQGTKVEIKR